(19) United States
(12) Patent Application Publication
(10) Pub. No.: US 2024/0109941 A1
(43) Pub. Date: Apr. 4, 2024

(54) COMPOSITIONS AND METHODS FOR TREATING LIBERIBACTER DISEASES AND OTHER BACTERIAL DISEASES

(71) Applicant: The Regents of the University of California, Oakland, CA (US)

(72) Inventors: Hailing Jin, Oakland, CA (US); Chien Yu Huang, Oakland, CA (US)

(21) Appl. No.: 17/754,712

(22) PCT Filed: Oct. 10, 2020
(86) PCT No.: PCT/US2020/055169

§ 371 Date: Apr. 8, 2022

Related U.S. Application Data
(60) Provisional application No. 62/914,243, filed on Oct. 11, 2019.

(51) Int. Cl. C07K 14/415; A01P 1/00; C12N 15/82

(52) U.S. Cl. CPC C07K 14/415; A01P 1/00; C12N 15/8281

(57) ABSTRACT

The disclosure provides stable antimicrobial (e.g., antibacterial or antifungal or both) peptides (SAMPs) that can be used in methods of preventing or treating a bacterial disease (e.g., a Liberibacter disease, such as citrus greening disease (also called Huanglongbing (HLB)) or potato Zebra Chip disease, and other bacterial diseases such as those caused by Agrobacterium tumefaciens (also known as Rhizobium radiobacter) and Pseudomonas syringae) in plants (e.g., citrus plants or potato plants).

### CROSS-REFERENCE TO RELATED APPLICATIONS

This application claims priority benefit of U.S. provisional application No. 62/914,243, filed Oct. 11, 2019, which is herein incorporated by reference for all purposes.

### BACKGROUND

Huanglongbing (HLB), also called citrus greening, is one of the most devastating citrus plant diseases. This citrus plant disease causes multibillion-dollar loss annually in the United States alone. According to research from the Institute of Food and Agricultural Sciences at the University of Florida, Florida has lost approximately 162,200 acres of citrus plants and 7,513 jobs since detection of HLB in Florida in 2005. The most recent forecasts from the National Association of Academies of Science predicted that citrus production from 2016 to 2017 is approximately 70% lower than peak production levels from 1997 to 1998. Moreover, HLB also spreads rapidly in Texas and California. Recently, more than 400 confirmed cases of HLB-infected trees have been reported in southern California. HLB is caused by the phloem-limited Gram-negative bacteria of the Liberibacter species, e.g., Candidatus Liberibacter species (e.g., Candidatus Liberibacter asiaticus (Ca. L. asiaticus)), which is transmitted by insects of the Psyllidae family, e.g., Asian citrus psyllids (ACP).

Another important disease cause by the Liberibacter species is Potato Zebra Chip (ZC) disease, also called Potato Zebra complex disease. ZC disease is associated with Candidatus Liberibacter solanacearum (Ca. L. solanacearum), which is transmitted by potato psyllids (e.g., Bactericera cockerelli). ZC disease reached epidemic level in northern Texas in 2006 and has spread to Arizona, California, Colorado, Idaho, Oregon, Kansas, Nebraska, and New Mexico. ZC disease has caused millions of dollars loss to the potato industry in the southwestern United States, particularly Texas. In addition to potato, other solanaceous crops, including tomato, eggplant and pepper, can also be infected. There exists a need in the art for innovative compositions and methods to treat diseases in plants caused by Liberibacter species (e.g., Candidatus Liberibacter species).

### SUMMARY

The disclosure provides a polypeptide comprising a stable antimicrobial peptide (SAMP) comprising a sequence of

HPX₁H(V/L)EX₂X₃X₄X₅X₆X₇X₈X₉X₁₀X₁₁X₁₂X₁₃X₁₄X₁₅DX₁₆X₁₇X₁₈X₁₉X₂₀X₂₁X₂₂X₂₃X₂₄X₂₅X₂₆ (SEQ ID NO:31), or a fragment thereof, wherein X₁ is S, A, or V; X₂ is Y or F; X₃, is A, S, or T; X₄ is N, A, or T; X₅ is L, T, I, S, or E; X₆ is F, M, or L; X₇ is L or S; X₈ is A, P, T, G, or S; X₉ is N, A, Q, S, or H; X₁₀ is L, I, or V; X₁₁ is E or D; X₁₂ is V, I, F, or T; X₁₃ is L, V, or I; X₁₄ is V, L, or I; X₁₅ is I, L, V, or F; X₁₆ is Y or F; X₁₇ is K or P; X₁₈ is P or T; X₁₉ is T, V, E, or Q; X₂₀ is T, L, K, S, or absent; X₂₁ is V, E, G, L, or absent; X₂₂ is R, K, G, S, N, or absent; X₂₃ is V, A, P, L, N, or absent; X₂₄ is P, S, or absent; X₂₅ is A or absent; and X₂₆ is A or absent, wherein the SAMP comprises a single α-helix structure.

In some embodiments of this aspect, the SAMP does not have the sequence of X₁GX₂X₃VSX₄ENX₅X₆QGFX₇HX₈FEX₉TFX₁₀SX₁₁EGX₁₂AEYX₁₃X₁₄ (SEQ ID NO:33), wherein X₁ is R, K, or W; X₂ is K or E; X₃ is N or D; X₄ is T or I; X₅ is L, F, or R; X₆ is H or Q; X₇ is P or T; X₈ is I, L, or V; X₉ is S or F; X₁₀ is E or D; X₁₁ is T or L; X₁₂ is V or I; X₁₃ is V or I; X₁₄ is S, A, or D. In certain embodiments, the SAMP has less than 67 amino acids (e.g., 66, 65, 64, 63, 62, 61, 60, 59, 58, 57, 56, 55, 50, 45, 40, 35, 35, 34, 33, 32, 31, 30, 29, 28, 27, 26, 25, 24, 23, 22, 21, 20, 19, 18, 17, 16, 15, 14, 13, 12, 11, or 10 amino acids).

In some embodiments of this aspect, the SAMP comprises a sequence of any one of

(SEQ ID NO: 17)
HPSHVEYANLFLANLEKVLVIDYKPTTVRV;
(SEQ ID NO: 18)
HPAHVEYANLFLANLEKVLVIDYKPTTVRV;
(SEQ ID NO: 19)
HPAHVEYANLFLANLEKVLVIDYKPTTERV;
(SEQ ID NO: 20)
HPSHVEFSATFSAAIEKIVLLDFPTVLGKAPAA;
(SEQ ID NO: 21)
HPVHVEFANLMLPQLEKVLVIDYKPEKVGP;
(SEQ ID NO: 22)
HPAHVEYANTLLPQLEKVLVIDYKPEKVGP;
(SEQ ID NO: 23)
HPVHVEYANTLLPQLEKFLIVDYKPQ;
(SEQ ID NO: 24)
HPAHVEYANILLTQLEKVLVIDYKPEKLSP;
(SEQ ID NO: 25)
HPVHVEFANTMLPQLEKVLIIDYKPQ;
(SEQ ID NO: 26)
HPAHVEYANSFLANLEKVLVIDYKPTTVRV;
(SEQ ID NO: 27)
HPAHVEYTNSFLANLEKVLVIDYKPTTVRV;
(SEQ ID NO: 28)
HPAHVEFATIFLGSLDKVLVIDYKPTSVSL;
(SEQ ID NO: 29)
HPAHVEFANEFLPALEKTLIIDYKPTSGNNS;
(SEQ ID NO: 30)
HPAHVEFANLFLSHVEKVIVEDYKPTTVRV,
and
(SEQ ID NO: 49)
HPVHLEFANLFLANLEKVLVIDYKPTTVRV;
or a fragment thereof.

In some embodiments of this aspect, the SAMP comprises 30 amino acids, the fifth amino acid in the 30-amino acid sequence is V; and X₁ is A, V, or S; X₂ is Y or F; X₃ is A or T; X₄ is N or T; X₅ is I, T, L, or S; X₆ is L, M, or F; X₇ is L; X₈ is T, P, G, S, or A; X₉ is Q, S, H, or N; X₁₀ is L or V; X₁₁ is E or D; X₁₂ is V; X₁₃ is L or I; X₁₄ is V; X₁₅ is I or F; X₁₆ is Y; X₁₇ is K, X₁₈ is P; X₁₉ is E or T; X₂₀ is K, S, or T; X₂₁ L, V, or E; X₂₂ is S, G, or R; X₂₃ is P, L, or V; X₂₄ is absent; X₂₅ is absent; and X₂₆ is absent. In certain embodiments, the SAMP comprises a sequence of any one of

(SEQ ID NO: 17)
HPSHVEYANLFLANLEKVLVIDYKPTTVRV;
(SEQ ID NO: 18)
HPAHVEYANLFLANLEKVLVIDYKPTTVRV;
(SEQ ID NO: 19)
HPAHVEYANLFLANLEKVLVIDYKPTTERV;
(SEQ ID NO: 21)
HPVHVEFANLMLPQLEKVLVIDYKPEKVGP;
(SEQ ID NO: 22)
HPAHVEYANTLLPQLEKVLVIDYKPEKVGP;
(SEQ ID NO: 24)
HPAHVEYANILLTQLEKVLVIDYKPEKLSP;
(SEQ ID NO: 26)
HPAHVEYANSFLANLEKVLVIDYKPTTVRV;
(SEQ ID NO: 27)
HPAHVEYTNSFLANLEKVLVIDYKPTTVRV;
(SEQ ID NO: 28)
HPAHVEFATIFLGSLDKVLVIDYKPTSVSL;
and
(SEQ ID NO: 30)
HPAHVEFANLFLSHVEKVIVFDYKPTTVRV.

In some embodiments of this aspect, X₁ is A or S; X₂ is Y; X₃ is A or T; X₄ is N; X₅ is L or S; X₆ is F; X₇ is L; X₈ is A; X₉ is N; X₁₀ is L; X₁₁ is E; X₁₂ is V; X₁₃ is L; X₁₄ is V; X₁₅ is I; X₁₆ is Y; Xv is K, X₁₇ is P; X₁₉ is E; X₂₀ is K; X₂₁ is V or L; X₂₂ is G or S; X₂₃ is P; X₂₄ is absent; X₂₅ is absent; and X₂₆ is absent. In certain embodiments, the SAMP comprises a sequence of any one of

(SEQ ID NO: 17)
HPSHVEYANLFLANLEKVLVIDYKPTTVRV;
(SEQ ID NO: 18)
HPAHVEYANLFLANLEKVLVIDYKPTTVRV;
(SEQ ID NO: 26)
HPAHVEYANSFLANLEKVLVIDYKPTTVRV;
and
(SEQ ID NO: 27)
HPAHVEYTNSFLANLEKVLVIDYKPTTVRV.

In some embodiments of this aspect, X₁ is A or V; X₂ is F or Y; X₃ is A; X₄ is N; X₅ is I, L, or T; X₆ is L or M; X₇ is L; X₈ is P or T; X₉ is Q; X₁₀ is L; X₁₁ is E; X₁₂ is V; X₁₃ is L; X₁₄ is V; X₁₅ is I; X₁₆ is Y; X₁₇ is K, X₁₈ is P; X₁₉ is T; X₂₀ is T; X₂₁ is V; X₂₂ is R; X₂₃ is V; X₂₄ is absent; X₂₅ is absent; and X₂₆ is absent. In certain embodiments, the SAMP comprises a sequence of any one of

(SEQ ID NO: 21)
HPVHVEFANLMLPQLEKVLVIDYKPEKVGP;
(SEQ ID NO: 22)
HPAHVEYANTLLPQLEKVLVIDYKPEKVGP;
and
(SEQ ID NO: 24)
HPAHVEYANILLTQLEKVLVIDYKPEKLSP.

In some embodiments of this aspect, X₁ is A; X₂ is F or Y; X₃ is A; X₄ is T or N; X₅ is I or L; X₆ is F; X₇ is L; X₈ is G, S or A; X₉ is S, H, or N; X₁₀ is L or V; X₁₁ is D or E; X₁₂ is V; X₁₃ is L or I; X₁₄ is V; X₁₅ is I or F; X₁₆ is Y; X₁₇ is K, X₁₈ is P; X₁₉ is T; X₂₀ is T or S; X₂₁ is V or E; X₂₂ is S or R; X₂₃ is L or V; X₂₄ is absent; X₂₅ is absent; and X₂₆ is absent. In certain embodiments, the SAMP comprises a sequence of any one of

(SEQ ID NO:19)
HPAHVEYANLFLANLEKVLVIDYKPTTERV;
(SEQ ID NO:28)
HPAHVEFATIFLGSLDKVLVIDYKPTSVSL;
and
(SEQ ID NO: 30)
HPAHVEFANLFLSHVEKVIVFDYKPTTVRV.

In some embodiments of this aspect, the SAMP comprises 26 amino acids and wherein X₁ is V; X₂ is Y or F; X₃ is A; X₄ is N; X₅ is T; X₆ is L or M; X₇ is L; X₈ is P; X₉ is Q; X₁₀ is L; X₁₁ is E; X₁₂ is F or V; X₁₃ is L; X₁₄ is I; X₁₅ is V or I; X₁₆ is Y; X₁₇ is K, X₁₈ is P; X₁₉ is Q; X₂₀ is absent; X₂₁ is absent; X₂₂ is absent; X₂₃ is absent; X₂₄ is absent; X₂₅ is absent; and X₂₆ is absent. In certain embodiments, the SAMP comprises a sequence of any one of

(SEQ ID NO:23)
HPVHVEYANTLLPQLEKFLIVDYKPQ;
and
(SEQ ID NO: 25)
HPVHVEFANTMLPQLEKVLIIDYKPQ.

In some embodiments of this aspect, the SAMP comprises a sequence of any one of

(SEQ ID NO:20)
HPSHVEFSATFSAAIEKIVLLDFPTVLGKAPAA;
and
(SEQ ID NO: 29)
HPAHVEFANEFLPALEKTLIIDYKPTSGNNS.

In another aspect, the disclosure provides a polypeptide comprising a stable antimicrobial peptide (SAMP) that comprises an α-helix and comprises a sequence of HPAHVEFATIFLX₁X₂X₃X₄KX₅X₆X₇X₈DYKPTX₉X₁₀X₁₁X₁₂X₁₃ (SEQ ID NO:32), or a fragment thereof, wherein X₁ is G, P, or S; X₂ is S, A, or H; X₃ is L or V; X₄ is D or E; X₅ is V or T; X₆ is L or I; X₇ is V or I; X₈ is I or F; X₉ is S or T; X₁₀ V or G; X₁₁ is S, N, or R; X₁₂ is L, N, or V; and X₁₃ is S or absent.

In some embodiments of this aspect, the SAMP comprises a sequence of any one of

(SEQ ID NO: 28)
HPAHVEFATIFLGSLDKVLVIDYKPTSVSL;
(SEQ ID NO: 29)
HPAHVEFANEFLPALEKTLIIDYKPTSGNNS;
and
(SEQ ID NO: 30)
HPAHVEFANLFLSHVEKVIVFDYKPTTVRV,
or a fragment thereof.

In some embodiments, the SAMP is at least 20 amino acids in length and comprises an α-helix structure. In some embodiments, the SAMP comprises between 20 and 24 amino acids (e.g., 20, 21, 22, 23, or 24 amino acids) and comprises an α-helix structure. In some embodiments, the SAMP comprises a sequence of any one of

(SEQ ID NO: 34)
SHVEYANLFLANLEKVIVIDYK;
(SEQ ID NO: 35)
AHVEYANLFLANLEKVIVIDYK;
(SEQ ID NO: 36)
AHVEYANLFLANLEKVLVIDYK;
(SEQ ID NO: 37)
SHVEFSATFSAAIEKIVLLDFP;
(SEQ ID NO: 38)
VHVEFANLMLPQLEKVLVIDYK;
(SEQ ID NO: 39)
AHVEYANTLLPQLEKVIVIDYK;
(SEQ ID NO: 40)
VHVEYANTLLPQLEKFLIVDYK;
(SEQ ID NO: 41)
AHVEYANILLTQLEKVIVIDYK;
(SEQ ID NO: 42)
VHVEFANTMLPQLEKVLIIDYK;
(SEQ ID NO: 43)
AHVEYANSFLANLEKVIVIDYK;
(SEQ ID NO: 44)
AHVEYTNSFLANLEKVIVIDYK;
(SEQ ID NO: 45)
AHVEFATIFLGSLDKVLVIDYK;
(SEQ ID NO: 46)
AHVEFANEFLPALEKTLIIDYK;
(SEQ ID NO: 47)
AHVEFANLFLSHVEKVIVEDYK;
(SEQ ID NO: 50)
VHLEFANLFLANLEKVIVIDYK;
(SEQ ID NO: 51)
SHVEFSATFSAAIEKIVLLDFPT;
and
(SEQ ID NO: 52)
AHVEYANSFLANLDKVLVIDYK;
or a fragment thereof.

In some embodiments, the amino acid sequence of the SAMP is:

(SEQ ID NO: 34)
SHVEYANLFLANLEKVLVIDYK;
(SEQ ID NO: 35)
AHVEYANLFLANLEKVLVIDYK;
(SEQ ID NO: 36)
AHVEYANLFLANLEKVLVIDYK;
(SEQ ID NO: 37)
SHVEFSATFSAAIEKIVLLDFP;
(SEQ ID NO: 38)
VHVEFANLMLPOLEKVIVIDYK;
(SEQ ID NO: 39)
AHVEYANTLLPQLEKVIVIDYK;
(SEQ ID NO: 40)
VHVEYANTLLPQLEKFLIVDYK;
(SEQ ID NO: 41)
AHVEYANILLTQLEKVIVIDYK;
(SEQ ID NO: 42)
VHVEFANTMLPOLEKVLIIDYK;
(SEQ ID NO: 43)
AHVEYANSFLANLEKVLVIDYK;
(SEQ ID NO: 44)
AHVEYTNSFLANLEKVIVIDYK;
(SEQ ID NO: 45)
AHVEFATIFLGSLDKVIVIDYK;
(SEQ ID NO: 46)
AHVEFANEFLPALEKTLIIDYK;
(SEQ ID NO: 47)
AHVEFANLFLSHVEKVIVEDYK;
(SEQ ID NO: 50)
VHLEFANLFLANLEKVIVIDYK;
(SEQ ID NO: 51)
SHVEFSATFSAAIEKIVLLDEPT;
or
(SEQ ID NO: 52)
AHVEYANSFLANLDKVIVIDYK.

In some embodiments, the SAMP has an α-helical structure and comprises (V/S/A)H(V/L)E(F/Y)(A/T/S)(N/A/T)(L/E/I/S/T)(F/L/M)(L/S)(A/S/P/G/T)(N/A/Q/S)(L/V/I) (E/D)K(V/I/T/F)(L/I/V)(V/L/I)(I/L/V/F)DYK (SEQ ID NO:53). In some embodiments, the SAMP has an α-helical structure and comprises (V/S/A)H(V/L)E(F/Y)(A/T/S)N(L/E/I/S/T)(F/L/M)(L/S)(A/S/P/G/T)(N/A/Q/S)(L/V/I)(E/D)K(V/I/T)(L/I/V)(V/L/I)(I/L/V)DYK (SEQ ID NO:54). In some embodiments, the SAMP has an α-helical structure and comprises (V/S/A)HVE(F/Y)AN(L/U/S/T)(F/L)(L/S)(A/P/)(N/A/Q/S)LEKV(L/I)(V/L/I)(I/L/V)DYK (SEQ ID NO:55). In some embodiments, the SAMP has an α-helical structure and comprises (V/S/A)HVE(F/Y)AN(L/I/S/T)(F/L)LA(N/Q)LEKV(L/I)(V/L/I)(I/L/V)DYK (SEQ ID NO:56).

In some embodiments, the SAMP has an α-helical structure and the sequence of the SAMP is (V/S/A)H(V/L)E(F/Y)(A/T/S)(N/A/T)(L/E/I/S/T)(F/L/M)(L/S)(A/S/P/G/T)((N/A/Q/S)(L/V/I) (E/D)K(V/I/T/F)(L/I/V)(V/L/I)(I/L/V/F)DYK (SEQ ID NO:53); (V/S/A)H(V/L)E(F/Y)(A/T/S)N(L/E/I/S/T)(F/L/M)(L/S)(A/S/P/G/T)(N/A/Q/S)(L/V/I)(E/D)K(V/I/T)(L/I/V)(V/L/I)(I/L/V)DYK (SEQ ID NO:54); (V/S/A)HVE(F/Y)AN(UlI/S/T)(F/L)(L/S)(A/P/)(N/A/Q/S)LEKV(L/I)XV/L/IXI/LUV)DYK (SEQ ID NO:55); or (V/S/A)HVE(F/Y)AN(L/I/S/T)(F/L)LA(N/Q)LEKV(L/I)(V/L/I)(I/L/V)DYK (SEQ ID NO:56).

In some embodiments, the SAMP has an α-helical structure and has at least 80% identity to any one of SEQ ID NOS:34-47, 50, 51, and 52. In some embodiments, the SAMP has an α-helical structure and has at least 85% identity to any one of SEQ ID NOS:34-47, 50, 51, and 52. In some embodiments, the SAMP has an α-helical structure and has at least 90% identity to any one of SEQ ID NOS:34-47, 50, 51, and 52. In some embodiments, the SAMP has an α-helical structure and has at least 95% identity to any one of SEQ ID NOS:34-47, 50, 51, and 52. In some embodiments, the SAMP comprises an amino acid sequence of any one of SEQ ID NOS:34-47, 50, 51, and 52. In some embodiments, the SAMP comprises the amino acid sequence of SEQ ID NO:34.

In some embodiments, the SAMP has an α-helical structure and has no more than four substitutions relative to any one of SEQ ID NOS:34-47, 50, 51, and 52. In some embodiments, the SAMP has an α-helical structure and has no more than three substitutions relative to any one of SEQ ID NOS:34-47, 50, 51, and 52. In some embodiments, the SAMP has an α-helical structure and has no more than two substitutions relative to any one of SEQ ID NOS:34-47, 50, 51, and 52. In some embodiments, the SAMP has an α-helical structure and has no more than one substitution relative to any one of SEQ ID NOS:34-47, 50, 51, and 52. For example, in some embodiments, a hydrophobic residue of the α-helix may be substituted with another hydrophobic residue.

In some embodiments, the SAMP is a heat stable (HS) peptide. In some embodiments, the SAMP is stable in plant extracts and/or in plant lysates.

In another aspect, the disclosure features an agricultural composition comprising a polypeptide comprising a SAMP described herein. The agricultural composition can further comprise at least one of an herbicide, an herbicide safener, a surfactant, a fungicide, a pesticide, a nematicide, a plant activator, a synergist, a plant growth regulator, an insect repellant, an acaricide, a molluscicide, or a fertilizer.

In another aspect, the disclosure features a nucleic acid molecule encoding a polypeptide comprising a SAMP described herein. Also provided is a polynucleotide comprising a promoter operably linked to the nucleic acid molecule (optionally where the promoter is heterologous to the nucleic acid molecule). The disclosure also features a cell comprising the nucleic acid molecule of the previous aspect. In some embodiments, the cell is a bacterial, yeast, plant, insect, or mammalian (e.g., human) cell. In particular embodiments, the cell is a plant cell.

In another aspect, the disclosure features a plant comprising a polypeptide comprising a SAMP described herein or the polynucleotide discussed above or the nucleic acid molecule encoding an isolated SAMP described herein. In some embodiments, the plant is a citrus plant or a solanaceous plant. In some embodiments, the plant is more tolerant to a bacterial pathogen compared to a control plant (otherwise identical) in which the SAMP is absent.

In another aspect, the disclosure features a plant comprising an in situ altered stable antimicrobial (e.g., antibacterial) peptide (SAMP) comprising at least one amino acid substitution corresponding to an amino acid at any one of positions X₁ to X₂₆ as set forth in SEQ ID NO:31, wherein the mutated SAMP provides Liberibacter disease (e.g., Huanglongbing (HLB)) resistance or Liberibacter disease (e.g., HLB) tolerance, Pseudomonas disease (e.g., bacterial canker or blast diseases) resistance or Pseudomonas disease tolerance, or Agrobacterium disease (e.g., Crown Gall disease or tumors) resistance or Agrobacterium disease tolerance to the plant.

In another aspect, the disclosure features an expression cassette comprising a promoter operably linked to a polynucleotide encoding a polypeptide comprising a SAMP described herein, wherein introduction of the expression cassette into a plant results in the plant having enhanced Liberibacter disease (e.g., HLB) resistance or Liberibacter disease (e.g., HLB) tolerance, Pseudomonas disease (e.g., bacterial canker or blast diseases) resistance or Pseudomonas disease tolerance, or Agrobacterium disease (e.g., Crown Gall disease or tumors) resistance or Agrobacterium disease tolerance. In some embodiments, the promoter of the expression cassette is heterologous to the polynucleotide. For example, when a promoter is said to be operably linked to a heterologous polynucleotide, it means that the polynucleotide is derived from one species whereas the promoter sequence is derived another, different species; or, if both are derived from the same species, the polynucleotide is not naturally associated with the promoter (e.g., the promoter is from a different gene in the same species, or has a modified sequence). In some embodiments, the promoter is inducible. In some embodiments, the promoter is plant tissue-specific (e.g., phloem-specific, tuber-specific, root-specific, stem-specific, trunk-specific, or leaf-specific. In some embodiments, the phloem-specific promoter is the sucrose transporter protein SUC2 promoter.

In another aspect, the disclosure features a transgenic plant comprising the expression cassette of the previous aspect, wherein the plant has enhanced Liberibacter disease (e.g., HLB) resistance or Liberibacter disease (e.g., HLB) tolerance, Pseudomonas disease (e.g., bacterial canker or blast diseases) resistance or Pseudomonas disease tolerance, or Agrobacterium disease (e.g., Crown Gall disease or tumors) resistance or Agrobacterium disease tolerance, as compared to a control plant lacking the expression cassette. In some embodiments, the transgenic plant is a citrus plant or a solanaceous plant.

In another aspect, the disclosure features a method of preventing or treating a Liberibacter disease (e.g., HLB), a Pseudomonas disease (e.g., bacterial canker or blast diseases), or an Agrobacterium disease (e.g., Crown Gall disease or tumors) in a plant by contacting the plant with a polypeptide comprising a SAMP described herein or an agricultural composition comprising an isolated SAMP. In some embodiments of these aspects, the isolated peptide or agricultural composition is injected, e.g., using microneedles, into the trunk of the plant. In some embodiments, the isolated peptide or agricultural composition is injected, e.g., using microneedles, into the stem of the plant. In other embodiments, the isolated peptide or agricultural composition is sprayed, e.g., by foliar spraying, onto the plant. In other embodiments, the isolated peptide or agricultural composition is applied to the root of the plant. In other embodiments, the isolated peptide or agricultural composition is applied to the plant by delivery to the roots, e.g., by irrigation, such as drip irrigation, soaking or hydroponics. In other embodiments, the isolated peptide or agricultural composition is applied by using a laser beam to enhance penetration of substances applied to citrus.

In another aspect, the disclosure features a method of preventing or treating potato Zebra Chip (ZC) disease in a plant by contacting the plant with a polypeptide comprising a SAMP described herein or an agricultural composition comprising an isolated SAMP. In yet another aspect, the disclosure features a method of preventing or treating a bacterial infection in a plant caused by bacteria in the genus Liberibacter (e.g., Candidatus Liberibacter species or Liberibacter crescens) by contacting the plant with an isolated SAMP described herein or an agricultural composition comprising an isolated SAMP. In still another aspect, the disclosure features a method of preventing or treating a bacterial infection in a plant caused by bacteria in the genus Agrobacterium (e.g., Agrobacterium tumefaciens species) by contacting the plant with an isolated SAMP described herein or an agricultural composition comprising an isolated SAMP. In still yet another aspect, the disclosure features a method of preventing or treating a bacterial infection in a plant caused by bacteria in the genus Pseudomonas (e.g., Pseudomonas syringae species) by contacting the plant with an isolated SAMP described herein or an agricultural composition comprising an isolated SAMP.

In another aspect, the disclosure features a method of inhibiting the growth of bacteria or killing bacteria in the genus Liberibacter (e.g., Candidatus Liberibacter species or Liberibacter crescens) in a plant by contacting the plant with a polypeptide comprising a SAMP described herein or an agricultural composition comprising an isolated SAMP. In yet another aspect, the disclosure features a method of inhibiting the growth of bacteria or killing bacteria in the genus Agrobacterium (e.g., Agrobacterium tumefaciens species) in a plant by contacting the plant with a polypeptide comprising a SAMP described herein or an agricultural composition comprising an isolated SAMP. In still yet another aspect, the disclosure features a method of inhibiting the growth of bacteria or killing bacteria in the genus Pseudomonas (e.g., Pseudomonas syringae species) in a plant by contacting the plant with a polypeptide comprising a SAMP described herein or an agricultural composition comprising an isolated SAMP.

In another aspect, the disclosure features a method of preventing or treating a Liberibacter disease (e.g. HLB) in a plant or preventing or treating a bacterial infection in a plant caused by bacteria in the genus Liberibacter (e.g., Candidatus Liberibacter species (e.g., Candidatus Liberibacter asiaticus, Candidatus Liberibacter africanus, and Candidatus Liberibacter americanus) or Liberibacter crescens) by introducing an expression cassette described herein (e.g., an expression cassette comprising a promoter operably linked to a polynucleotide encoding an isolated stable antimicrobial (e.g., antibacterial) peptide described herein) into the plant. In another aspect, the disclosure features a method of preventing or treating potato ZC disease in a plant or preventing or treating a bacterial infection in a plant caused by bacteria in the genus Liberibacter (e.g., Candidatus Liberibacter species (e.g., Candidatus Liberibacter solanacearum (Ca. L. solanacearum)) or Liberibacter crescens) by introducing an expression cassette described herein (e.g., an expression cassette comprising a promoter operably linked to a polynucleotide encoding an isolated SAMP described herein) into the plant.

In yet another aspect, the disclosure features a method of preventing or treating a Pseudomonas disease (e.g., bacterial canker or blast diseases) in a plant or preventing or treating a bacterial infection in a plant caused by bacteria in the genus Pseudomonas (e.g., Pseudomonas syringae species) by introducing an expression cassette described herein (e.g., an expression cassette comprising a promoter operably linked to a polynucleotide encoding an isolated stable antimicrobial (e.g., antibacterial) peptide described herein) into the plant.

In still yet another aspect, the disclosure features a method of preventing or treating a Agrobacterium disease (e.g., Crown Gall disease or tumors) in a plant or preventing or treating a bacterial infection in a plant caused by bacteria in the genus Agrobacterium (e.g., Agrobacterium tumefaciens species) by introducing an expression cassette described herein (e.g., an expression cassette comprising a promoter operably linked to a polynucleotide encoding an isolated stable antimicrobial (e.g., antibacterial) peptide described herein) into the plant.

In another aspect, the disclosure features a method of inhibiting the growth of bacteria or killing bacteria in the genus Liberibacter (e.g., Candidatus Liberibacter species or Liberibacter crescens) in a plant by introducing an expression cassette described herein (e.g., an expression cassette comprising a promoter operably linked to a polynucleotide encoding an isolated stable antimicrobial (e.g., antibacterial) peptide described herein) into the plant.

In another aspect, the disclosure features a method of inhibiting the growth of bacteria or killing bacteria in the genus Agrobacterium (e.g., Agrobacterium tumefaciens species) in a plant by introducing an expression cassette described herein (e.g., an expression cassette comprising a promoter operably linked to a polynucleotide encoding an isolated stable antimicrobial (e.g., antibacterial) peptide described herein) into the plant.

In yet another aspect, the disclosure features a method of inhibiting the growth of bacteria or killing bacteria in the genus Pseudomonas (e.g., Pseudomonas syringae species) in a plant by introducing an expression cassette described herein (e.g., an expression cassette comprising a promoter operably linked to a polynucleotide encoding an isolated stable antimicrobial (e.g., antibacterial) peptide described herein) into the plant.

In another aspect, the disclosure features a method of producing a plant having enhanced Liberibacter disease (e.g., HLB) resistance or Liberibacter disease (e.g., HLB) tolerance by introducing an isolated stable antimicrobial (e.g., antibacterial) peptide described herein or an expression cassette comprising a promoter operably linked to a polynucleotide encoding an isolated stable antimicrobial (e.g., antibacterial) peptide into a plurality of plants; and selecting a plant that comprises the isolated peptide or expresses the polynucleotide from the plurality of plants.

In another aspect, the disclosure features a method of producing a plant having enhanced potato ZC disease resistance or potato ZC disease tolerance by introducing a polypeptide comprising a SAMP described herein or an expression cassette comprising a promoter operably linked to a polynucleotide encoding an isolated SAMP into a plurality of plants; and selecting a plant that comprises the isolated peptide or expresses the polynucleotide from the plurality of plants.

In another aspect, the disclosure features a method of producing a plant having enhanced Pseudomonas disease (e.g., bacterial canker or blast diseases) resistance or Pseudomonas disease (e.g., bacterial canker or blast diseases) tolerance by introducing an isolated stable antimicrobial (e.g., antibacterial) peptide described herein or an expression cassette comprising a promoter operably linked to a polynucleotide encoding an isolated stable antimicrobial (e.g., antibacterial) peptide into a plurality of plants; and selecting a plant that comprises the isolated peptide or expresses the polynucleotide from the plurality of plants.

In another aspect, the disclosure features a method of producing a plant having enhanced Agrobacterium disease (e.g., Crown Gall disease or tumors) resistance or Agrobacterium disease (e.g., Crown Gall disease or tumors) tolerance by introducing an isolated stable antimicrobial (e.g., antibacterial) peptide described herein or an expression cassette comprising a promoter operably linked to a polynucleotide encoding an isolated stable antimicrobial (e.g., antibacterial) peptide into a plurality of plants; and selecting a plant that comprises the isolated peptide or expresses the polynucleotide from the plurality of plants.

In another aspect, the disclosure features a method of producing a plant having enhanced Liberibacter disease (e.g., HLB) resistance or Liberibacter disease (e.g., HLB) tolerance (i.e., enhanced resistance or tolerance to a bacterial infection caused by bacteria in the genus Liberibacter (e.g., Candidatus Liberibacter species (e.g., Candidatus Liberibacter asiaticus, Candidatus Liberibacter africanus, and Candidatus Liberibacter americanus) or Liberibacter crescens)) by introducing a mutation into a polynucleotide in the plant, wherein the mutated polynucleotide encodes an isolated stable antimicrobial (e.g., antibacterial) peptide described herein (e.g., an isolated SAMP having at least 80% sequence identity, or at least 90% sequence identity, to the sequence of any one of SEQ ID NOS:17-30, 49 and 34-47). In another aspect, the disclosure features a method of producing a plant having enhanced potato ZC disease resistance or potato ZC disease tolerance (i.e., enhanced resistance or tolerance to a bacterial infection caused by bacteria in the genus Liberibacter (e.g., Candidatus Liberibacter species (e.g., Candidaus Liberibacter solanacearum (Ca. L. solanacearum)) or Liberibacter crescens) by introducing a mutation into a polynucleotide in the plant, wherein the mutated polynucleotide encodes a polypeptide comprising a SAMP described herein (e.g., an isolated SAMP having at least 80% sequence identity, or at least 90% sequence identity, to the sequence of any one of SEQ ID NOS:17-30, 49, and 34-47). In some embodiments of these aspects, the introducing occurs in situ in the genome of a plant cell. In particular embodiments, the introducing comprises clustered regularly interspaced short palindromic repeats (CRISPR)/Cas genome editing. In some embodiments of these aspects, the plant is a citrus plant or a solanaceous plant (e.g., a potato plant).

In still another aspect, the disclosure features a method of producing a plant having enhanced Agrobacterium disease resistance or Agrobacterium disease tolerance (i.e., enhanced resistance or tolerance to a bacterial infection caused by bacteria in the genus Agrobacterium (e.g., Crown Gall disease or tumors caused by Agrobacterium strains)) by introducing a mutation into a polynucleotide in the plant, wherein the mutated polynucleotide encodes a polypeptide comprising a SAMP described herein (e.g., an isolated SAMP having at least 80% sequence identity, or at least 90% sequence identity, to the sequence of any one of SEQ ID NOS:17-30, 49, and 34-47). In some embodiments of these aspects, the introducing occurs in situ in the genome of a plant cell. In particular embodiments, the introducing comprises clustered regularly interspaced short palindromic repeats (CRISPR)/Cas genome editing. In some embodiments of these aspects, the plant is a dicot. In some embodiments of these aspects, the plant is eudicot plant.

In still another aspect, the disclosure features a method of producing a plant having enhanced Pseudomonas disease resistance or Pseudomonas disease tolerance (i.e., enhanced resistance or tolerance to a bacterial infection caused by bacteria in the genus Pseudomonas (e.g., bacterial canker or blast diseases caused by Pseudomonas strains)) by introducing a mutation into a polynucleotide in the plant, wherein the mutated polynucleotide encodes a polypeptide comprising a SAMP described herein (e.g., an isolated SAMP having at least 80% sequence identity, or at least 90% sequence identity, to the sequence of any one of SEQ ID NOS:17-30, 49, and 34-47). In some embodiments of these aspects, the introducing occurs in situ in the genome of a plant cell. In particular embodiments, the introducing comprises clustered regularly interspaced short palindromic repeats (CRISPR)/Cas genome editing. In some embodiments of these aspects, the plant is a monocot or dicot plant (e.g., a tomato plant).

In any of the compositions or methods described in the present disclosure, the plant can species be from the genus Citrus (e.g., Citrus maxima, Citrus medica, Citrus micrantha, Citrus reticulate, Citrus aurantiffolia, Citrus aurantium, Citrus latifolia, Citrus limon, Citrus limonia, Citrus paradise, Citrus sinensis, and Citrus tangerine) or species from the family Solanaceae (e.g., Solanum spp., Capsicum spp., and Nicotiana spp.). Species from the genus Solanum include, e.g., Solanum tuberosum, Solanum lycopersicum, Solanum melongena, Solanum aviculare, Solanum capsicastrum, Solanum crispum, Solanum laciniatum, Solanum laxum, Solanum pseudocapsicum, Solanum rantonnetii, Solanum seaforthianum, and Solanum wendlandii. Species from the genus Capsicum include, e.g., Capsicum annuum, Capsicum baccatum, Capsicum campylopodium, Capsicum cardenasii, Capsicum chacoense, Capsicum cornutum, Capsicum dusenii, Capsicum eximium, Capsicum friburgense, Capsicum frutescens, Capsicum geminifolium, Capsicum havanense, Capsicum lanceolatum, Capsicum lycianthoides, Capsicum minutilorum, Capsicum mositicum, Capsicum pubescens, Capsicum recurvatum, Capsicum schottianum, Capsicum spina-alba, Capsicum tovarii, and Capsicum villosum. Species from the genus Nicotiana include, e.g., Nicotiana acuminate, Nicotiana benthamiana, Nicotiana glauca, Nicotiana longiflora, Nicotiana rustica, Nicotiana tabacum, and Nicotiana occidentalis.

In particular embodiments, the plant is selected from the group consisting of Citrus reticulata, Citrus sinensis, Citrus clementina, Capsicum annuum, Solanum tuberosum, Solanum lycopersicum, Solanum melongena, and Nitotiana benthamiana. In particular embodiments, the plant is a sweet orange plant (Citrus sinensis). In particular embodiments, the plant is a clementine plant (Citrus clementina). In particular embodiments, the plant is a potato plant (Solanum tuberosum). In some embodiment, the plant is a vegetable- or fruit-producing plant.

In any of the aspects of the disclosure described herein, in some embodiments, the SAMP is a heat stable (HS) peptide.

Furthermore, in any of the aspects of the disclosure described herein, in some embodiments, the SAMP can also provide resistance or tolerance to bacterial diseases caused by other bacterial pathogens, such as Agrobacterium tumefaciens (also known as Rhizobium radiobacter) and Pseudomonas syringae.

### Definitions

As used herein, the term “Liberibacter disease” refers to a disease, such as an infection, caused by bacteria in the genus Liberibacter (e.g., Candidatus Liberibacter species or Liberibacter crescens). A Liberibacter disease can infect plants such as citrus plants (e.g., orange, grapefruit, tangerine, lemon, line, key line, papeda, citron, and pomelo) and solanaceous plants (e.g., potato, tomato, eggplant, and pepper). Huanglongbing (HLB) is a type of Liberibacter disease that infects citrus plants.

As used herein, the terms “citrus greening disease” and “Huanglongbing (HLB)” refer to a bacterial infection of plants (e.g., citrus plants) caused by bacteria in the genus Liberibacter (e.g., Candidatus Liberibacter species (e.g., Candidatus Liberibacter asiaticus, Candidatus Liberibacter africanus, and Candidatus Liberibacter americanus) or Liberibacter crescens). The infection is vectored and transmitted by the Asian citrus psyllid, Diaphorina citri, and the African citrus psyllid, Trioza erytreae. Three different types of HLB are currently known: the heat-tolerant Asian form, and the heat-sensitive African and American forms.

As used herein, the term “Potato Zebra Chip (ZC) disease” refers to a bacterial infection of plants (e.g., potato plants) caused by bacteria in the genus Liberibacter (e.g., Candidatus Liberibacter species (e.g., Candidatus Liberibacter solanacearum (Ca. L. solanacearum)) or Liberibacter crescens). The infection is vectored and transmitted by potato psyllids (e.g., Bactericera cockerelli).

As used herein, the term “Agrobacterium disease” refers to a disease, such as an infection, caused by bacteria in the genus Agrobacterium (e.g., Agrobacterium tumefaciens species, also known as Rhizobium radiobacter). Agrobacterium diseases can comprise Crown Gall disease, or tumors, in more than 140 eudicot species.

As used herein, the term “Pseudomonas disease” refers to a disease, such as an infection, caused by bacteria in the genus Pseudomonas (e.g., Pseudomonas syringae species). Pseudomonas diseases can comprise bacterial canker or blast diseases on many dicot and monocot crops (e.g., Tomato Bacterial Speck, Tomato Bacterial Spot, and Tomato Bacterial Canker).

As used herein, the term “disease resistance” refers to the ability of a plant to not be affected by a Liberibacter disease (e.g., HLB), Agrobacterium disease, or Pseudomonas disease; or infection by Liberibacter bacteria (e.g., Candidatus Liberibacter species or Liberibacter crescens), Agrobacterium bacteria (e.g., Agrobacterium tumefaciens species), or Pseudomonas bacteria (e.g., Pseudomonas syringae species).

As used herein, the term “disease tolerance” refers to the ability of a plant to continuously grow and survive despite being infected by bacteria (e.g. gram-negative bacteria, such as Liberibacter bacteria. Agrobacterium bacteria or Pseudomonas bacteria).

As used herein, the term “Liberibacter disease resistance” refers to the ability of a plant to not be affected by a Liberibacter disease (e.g., HLB) or infection by Liberibacter bacteria (e.g., Candidatus Liberibacter species or Liberibacter crescens).

As used herein, the term “Liberibacter disease tolerance” refers to the ability of a plant to continuously grow and survive despite being infected by Liberibacter bacteria (e.g., Candidatus Liberibacter species or Liberibacter crescens) or having a Liberibacter disease (e.g., HLB). In some embodiments, a plant with a Liberibacter disease (e.g., HLB) can show minor symptoms of the disease, such as yellowing of leaves, blotchy mottle of the leaves, zinc-deficiency-like mottle, chlorosis, and reduced fruit yield, but is still able to grow or produce fruit despite the infection.

As used herein, the term “potato ZC disease resistance” refers to the ability of a plant to not be affected by potato ZC disease or infection by Liberibacter (e.g., Candidatus Liberibacter species (e.g., Candidatus Liberibacter solanacearum (Ca. L. solanacearum)) or Liberibacter crescens) bacteria.

As used herein, the term “potato ZC disease tolerance” refers to the ability of a plant to continuously grow and survive despite being infected by Liberibacter (e.g., Candidatus Liberibacter species (e.g., Candidatus Liberibacter solanacearum (Ca. L. solanacearum)) or Liberibacter crescens) bacteria or having potato ZC disease. In some embodiments, a plant with potato ZC disease can show minor symptoms of potato ZC disease, such as chlorosis, leaf scorching, swollen nodes, vascular tissue browning, curled leaves, collapsed stolons, enlarged lenticels, vascular tissue browning, medullary ray discoloration, and necrotic flecking of tuber tissue, but is still able to grow or produce potato despite the infection.

As used herein, the term “Agrobacterium disease resistance” refers to the ability of a plant to not be affected by an Agrobacterium disease (e.g., Crown Gall disease) or infection by Agrobacterium bacteria (e.g., Agrobacterium tunefaciens species, also known as Rhizobium radiobacter).

As used herein, the term “Agrobacterium disease tolerance” refers to the ability of a plant to continuously grow and survive despite being infected by Agrobacterium bacteria (e.g., Agrobacterium tumefaciens species, also known as Rhizobium radiobacter) or having an Agrobacterium disease (e.g., Crown Gall disease).

As used herein, the term “Pseudomonas disease resistance” refers to the ability of a plant to not be affected by a Pseudomonas disease (e.g., Tomato Bacterial Speck, Tomato Bacterial Spot, and Tomato Bacterial Canker) or infection by Pseudomonas bacteria (e.g., Agrobacterium tumefaciens species).

As used herein, the term “Pseudomonas disease tolerance” refers to the ability of a plant to continuously grow and survive despite being infected by Pseudomonas bacteria (e.g., Agrobacterium tumefaciens species) or having a Pseudomonas disease (e.g., Tomato Bacterial Speck, Tomato Bacterial Spot, and Tomato Bacterial Canker).

As used herein, the term “stable antimicrobial peptides” or “SAMPs” refers to peptides identified in plants that are Liberibacter disease-resistant/tolerant (e.g., HLB-resistant/tolerant) or fragments thereof. Such peptides are expressed at a higher level in Liberibacter disease-resistant/tolerant (e.g., HLB-resistant/tolerant) plants than Liberibacter disease-susceptible (e.g., HLB-susceptible) plants. These SAMPs can be injected into plants to prevent or treat a Liberibacter disease (e.g., HLB). The SAMPs disclosed herein can be naturally occurring or synthetic. In some embodiments, the SAMPs disclosed herein only has a single α-helix. In some embodiments, the SAMPs disclosed herein has less than 67 amino acids (e.g., 66, 65, 64, 63, 62, 61, 60, 59, 58, 57, 56, 55, 50, 45, 40, 35, 35, 34, 33, 32, 31, 30, 29, 28, 27, 26, 25, 24, 23, 22, 21, 20, 19, 18, 17, 16, 15, 14, 13, 12, 11, or 10 amino acids). In some embodiments, the SAMPs comprise at least 20 amino acids (e.g., between 20 and 24 amino acids, e.g., 20, 21, 22, 23, or 24 amino acids). The SAMPs disclosed herein do not have the sequence of SEQ ID NO:33. In some embodiments, the SAMPs disclosed herein have antibacterial or antifungal or both properties. In some embodiments, the SAMPs disclosed herein are heat stable (e.g., heat stable (HS) peptides). In some embodiments, the SAMPs disclosed herein are also stable in plant extracts. In further embodiments, the SAMPs disclosed herein are also stable in plant lysates (e.g., citrus lysates).

As used herein, the term “agricultural composition” refers to a composition formulated for application to a plant or plant part (e.g., seed, cutting, shoots, etc.). An agricultural composition is typically in liquid form, e.g., for application by spraying or soaking, but can be in a powder for rehydration or application (dusting or dry coating), or gaseous form (e.g. for enclosed environments). The agricultural composition can be concentrated, e.g., for dilution or water or other solvent. An agricultural composition can also include more than one active ingredient, e.g., a SAMP (e.g., an HS peptide) described herein, alone or in combination with a fungicide, herbicide, fertilizer, etc.

As used herein, the term “treat” or “treating” a Liberibacter disease (e.g, an HLB or a potato ZC disease) in plants refers to the reduction or eradication of symptoms caused by the Liberibacter disease by methods described herein. Symptoms of HLB include, but are not limited to, yellowing of leaves, blotchy mottle of the leaves, zinc-deficiency-like mottle, severe chlorosis, and reduced fruit yield. Symptoms of potato ZC disease include, but are not limited to, chlorosis, leaf scorching, swollen nodes, vascular tissue browning, curled leaves, collapsed stolons, enlarged lenticels, vascular tissue browning, medullary ray discoloration, and necrotic flecking of tuber tissue. In some embodiments, the disclosed methods may not necessarily result in eradication or cure of the Liberibacter disease (e.g., HLB or potato ZC disease), but can significantly reduce the symptoms caused by the disease.

As used herein, the term “prevent” or “preventing” a Liberibacter disease (e.g., an HLB or a potato ZC disease) in plants refers protecting a plant that is at risk for the disease from developing the disease, or decreasing the risk that a plant can develop the disease. A plant can be contacted with a SAMP (e.g., an HS peptide) described herein before the plant develops the disease, or shows signs of the disease.

As used herein, the term “plant” includes whole plants, shoot vegetative organs/structures (e.g., leaves, stems and tubers), roots, flowers and floral organs/structures (e.g., bracts, sepals, petals, stamens, carpels, anthers and ovules), seed (including embryo, endosperm, and seed coat) and fruit (the mature ovary), plant tissue (e.g., vascular tissue, ground tissue, and the like) and cells (e.g., guard cells, egg cells, and the like), and progeny of same. In some embodiments, the plant is a transgenic or genome-edited rootstock, a non-transgenic or non-genome-edited scion, a transgenic or genome-edited scion, or a non-transgenic or non-genome-edited rootstock. The term “plant” also includes transgenic or genome-edited plant cells, tissue, or parts grafted into or onto a separate plant. Plants that can be treated as described herein include, e.g., citrus plants (e.g., orange, grapefruit, tangerine, lemon, line, key line, papeda, citron, and pomelo) and solanaceous plants (e.g., potato, tomato, eggplant, and pepper).

As noted above, the term “plant” also includes naturally occurring mutants and genetically modified plants. A “genetically modified plant” or “transgenic plant” refers to one whose genome has been manipulated so that it is different than a wild-type plant of the same species, variety or cultivar, e.g., to add a gene or genetic element, remove a gene or genetic element, mutate a gene or genetic element, change chromatin structure, change gene or protein expression levels, etc. A transgenic plant can contain an expression vector or cassette. The expression cassette typically comprises a polypeptide-encoding sequence or a modulating nucleic acid (e.g., an antisense, an siRNA or ribozyme) operably linked (i.e., under regulatory control of) to an appropriate inducible or constitutive regulatory sequences that allow for the expression of a polypeptide or modulating nucleic acid. The expression cassette can be introduced into a plant by transformation or by breeding after transformation of a parent plant. Such methods can be used in a whole plant, including seedlings and mature plants, as well as to a plant part, such as seed, fruit, leaf, or root, plant tissue, plant cells or any other plant material, e.g., a plant explant, as well as to progeny thereof, and to in vitro systems that mimic biochemical or cellular components or processes in a cell. In the context of the present disclosure, genetically modified plants can include genetic modifications in a gene encoding a SAMP (e.g., an HS peptide). In some embodiments, the modified gene in the genetically modified plant can encode a SAMP (e.g., an HS peptide) described herein (e.g., a SAMP having at least 80% sequence identity, or at least 90% sequence identity, or at least one amino acid substitution, e.g., 1, 2, 3, 4, 5, 6, 7, or 8 substitutions, relative to the sequence of any one of SEQ ID NOS:17-30, and 49; or SEQ ID NOS:34-47 and 50-52).

In the context of an “exogenous” polypeptide applied or contacted with a plant, the term “exogenous” means that the polypeptide did not occur naturally (e.g., by native genomic expression) in the plant, without human intervention. Embodiments of an “exogenous” polypeptide include, e.g., a polypeptide that is produced by in vitro fermentation or by production in cells or tissues or by synthesis and that is then provided to the plant (e.g., in an agricultural composition delivered by injection, dipping, spraying, soaking, irrigation), or a polypeptide expressed in cells (e.g., bacterial cells) or tissue (e.g., transgenic or genome-edited plant tissue) that is then provided to the plant (e.g., by extraction and delivery in an agricultural composition, or by grafting), or by providing to the plant a recombinant construct that expresses the exogenous polypeptide in the plant, or by modifying the plant's genome (e.g., by genome editing methods such as CRISPR/Cas, TALENs, base editing, or prime editing) resulting in the plant's expression of the exogenous polypeptide.

An “expression cassette” refers to a nucleic acid construct that, when introduced into a host cell, results in transcription and/or translation of an RNA or polypeptide, respectively.

As used herein, the term “polynucleotide” refers to an oligonucleotide, or nucleotide, and fragments or portions thereof, and to DNA or RNA of genomic or synthetic origin, which can be single- or double-stranded, and represent the sense or anti-sense strand. A single polynucleotide is translated into a single polypeptide.

As used herein, the terms “peptide” and “polypeptide” are used interchangeably and describe a single polymer in which the monomers are amino acid residues which are joined together through amide bonds. A polypeptide is intended to encompass any amino acid sequence, either naturally occurring, recombinant, or synthetically produced.

As used herein, the term “substantial identity” or “substantially identical,” used in the context of nucleic acids or polypeptides, refers to a sequence that has at least 50% sequence identity with a reference sequence. Alternatively, percent identity can be any integer from 50% to 100%. In some embodiments, a sequence is substantially identical to a reference sequence if the sequence has at least 50%, 55%, 60%, 65%, 70%, 75%, 80%, 85%, 90%, 91%, 92%, 93%, 94%, 95%, 96%, 97%, 98%, or 99% sequence identity to the reference sequence as determined using the methods described herein; preferably BLAST using standard parameters, as described below. Embodiments of the present disclosure provide for SAMPs (e.g., HS peptides) that are substantially identical to any of SEQ ID NOS: 17-30, 49, and 34-47, or a fragment thereof.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A comparison window includes reference to a segment of any one of a number of contiguous positions, e.g., a segment of at least 10 residues. In some embodiments, the comparison window has from 10 to 600 residues, e.g., about 10 to about 30 residues, about 10 to about 20 residues, about 50 to about 200 residues, or about 100 to about 150 residues, in which a sequence can be compared to a reference sequence of the same number of contiguous positions after the two sequences are optimally aligned.

Algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1977) Nucleic Acids Res. 25: 3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see. e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, an amino acid sequence is considered similar to a reference sequence if the smallest sum probability in a comparison of the test amino acid sequence to the reference amino acid sequence is less than about 0.01, more preferably less than about 10⁻⁵, and most preferably less than about 10⁻²⁰.

### BRIEF DESCRIPTION OF THE DRAWINGS

FIG. 1A: Structural motifs of an exemplary SAMP, CghSAMPa, found in Eremocitrus glauca x citrus sp. A polypeptide containing “helix 2”, as explained in Example 1, was found to be a functional SAMP.

FIG. 1B: Treatment of Liberibacter crescens with the full length CghSAMPa (SEQ ID NO:1. SAMP), CghSAMPa with N-terminus and C-terminus deletion (SAMPΔNΔC, SEQ ID NO:57), a polypeptide containing CghSAMPa helix 1 (SAMP-helix 1, SEQ ID NO:58), or a polypeptide containing CghSAMPa helix 2 (SAMP-helix 2, SEQ ID NO:34) for 2 hours.

FIGS. 2A and 2B: As explained in Example 2, FIGS. 2A and 2B provide data illustrating extended treatment time and increased concentration of CghSAMPa (SEQ ID NO:1). CghSAMPa helix 2 (SEQ ID NO:34) exhibited similar antibacterial activity compared to full length CghSAMPa. SAMP=CghSAMPa (SEQ ID NO:1); SAMPaH2=CghSAMPa-helix 2 (SEQ ID NO:34)

FIGS. 3A-3C: SAMP-helix 2 (SEQ ID NO:34) induced a defense response in Carrizo rootstock.

FIGS. 4A-4C: FIGS. 4A-4C illustrate that the antibacterial motif of CghSAMPa, helix 2 of CghSAMPa (referred to in FIG. 4A-4C as MaSSAMP-helix2), forms polymers. FIG. 4A shows the structure prediction of CghSAMPa (referred to as MaSSAMP in FIGS. 4A-4C) and MaSSAMP-helix2 by swiss-model. The hydrophobic residues are darker in color. FIG. 4B shows a helical wheel prediction diagram of MaSSAMP-helix2. The hydrophobic residues are shown in squares. FIG. 4C depicts a native PAGE gel shows that MaSSAMP and MaSSAMP-helix2 formed hexamers in the native gel.

FIG. 5, provides data illustrating that Gram-positive B. thuringiensis is highly tolerant to CghSAMPa. (SAMP)

FIG. 6A-6C provide data illustrating that B. thuringiensis and E. coli can be used for CghSAMPa (SAMP) production.

### DETAILED DESCRIPTION OF THE EMBODIMENTS

### I. Introduction

Citrus greening disease or “Huanglongbing” (HLB), caused by bacteria Candidatus Liberibacter, is a type of Liberibacter disease that specifically infects citrus plants. HLB is one of the most destructive diseases of citrus. Liberibacter (e.g., Candidatus Liberibacter species (Ca. Liberibacter or Ca. L.) or Liberibacter crescens) is a Gram-negative bacterial pathogen restricted to the phloem. HLB has caused in a significant reduction in citrus quality and quantity, resulting in billions of dollars in losses of citrus products every year, and seriously impacts the viability of the citrus industry. Current methods of treating HLB mainly involve removal of infected plants and chemical treatment against the insect vector and only led to partial control of the disease. No sustainable disease control methods for HLB have been found. The expansive and fast spread of the disease to multiple locations has already made complete removal of the infected trees an impractical strategy.

To identify and characterize important citrus defense regulators, a comparative analysis of small RNAs (sRNAs) and sRNA target genes between HLB-resistant and HLB-tolerant hybrid varieties and HLB-susceptible varieties. Several citrus defense regulators that uniquely respond to Ca. L. infection in HLB-resistant and HLB-tolerant hybrid varieties, but not in HLB-susceptible varieties were identified. Among the identified citrus defense regulators, putative antibacterial genes encoding stable antimicrobial proteins were found to express at a much higher level in the HLB-resistant and HLB-tolerant hybrid varieties than in HLB-susceptible varieties. The HLB-resistant and HLB-tolerant hybrid rootstocks are from completely different geographic and genetic backgrounds.

After the identification of these stable antimicrobial proteins that provide HLB-resistance or HLB-tolerance, a functional analysis of these stable antimicrobial proteins was performed. The result showed that polypeptides containing a SAMP that only has the second α-helix of the full length SAMP can effectively inhibit or kill Liberibacter species (e.g., Liberibacter crescens) and achieve plant protection.

In some embodiments, the SAMPs described herein that only have a single α-helix.

The SAMPs described herein do not have the sequence of SEQ ID NO:33. In some embodiments, the SAMPs described herein have less than 67 amino acids (e.g., 66, 65, 64, 63, 62, 61, 60, 59, 58, 57, 56, 55, 50, 45, 40, 35, 35, 34, 33, 32, 31, 30, 29, 28, 27, 26, 25, 24, 23, 22, 21, 20, 19, 18, 17, 16, 15, 14, 13, 12, 11, or 10 amino acids). The SAMPs can comprises a sequence of any one of SEQ ID NOS:17-32, or 49. In some embodiments, the SAMPs comprise at least 20 amino acids (e.g., between 20 and 24 amino acids, e.g., 20, 21, 22, 23, or 24 amino acids). The SAMPs can comprises a sequence of any one of SEQ ID NOS:34-47, 50, 51, and 52. The disclosure also includes agricultural compositions containing such SAMPs, plants comprising such SAMPs, transgenic plants expressing such SAMPs, methods of using such SAMPs to prevent or treat bacterial diseases, such as those caused by Gram-negative bacteria, e.g., a Liberibacter disease (e.g., an HLB or a potato ZC disease), bacterial diseases caused by Agrobacterium tumefaciens (also known as Rhizobium radiobacter), and bacterial diseases caused by Pseudomonas syringae, and methods of producing plants that comprise such SAMPs or express such SAMPs. The SAMPs disclosed herein are found to be effective when only containing a single α-helix, while full length SAMPs contain two α-helices. The SAMPs disclosed herein can also provide plants with resistance or tolerance to bacterial diseases, such as those caused by Gram-negative bacteria, e.g., bacterial diseases caused by Liberibacter species, Agrobacterium tumefaciens, and Pseudomonas syringae strains.

### II. Stable Antimicrobial Peptides (SAMPs)

The disclosure provides stable antimicrobial (e.g., antibacterial) peptides (SAMPs) that can be injected into plants to prevent or treat a bacterial disease, such as a Gram-negative bacterial disease, e.g., a Liberibacter disease (e.g., HLB). The SAMPs disclosed herein can also be used to prevent or treat bacterial diseases caused by other bacterial pathogens, such as Agrobacterium tumefaciens (also known as Rhizobium radiobacter) and Pseudomonas syringae strains. The plants can also be genetically modified to express one or more of the SAMPs described herein. In some embodiments, the SAMPs disclosed herein are heat stable (e.g., heat stable (HS) peptides). In some embodiments, the SAMPs disclosed herein are also stable in plant extracts. In further embodiments, the SAMPs disclosed herein are also stable in plant lysates (e.g. citrus lysates). The SAMPs disclosed herein are found to be effective when only containing a single α-helix, while full length SAMPs contain two α-helices. Table 1 below provides amino acid sequences of full length SAMPs found in various hybrid plants, citrus plants, and solanaceous plants and their functional fragments that contain only a single α-helix (the second helix of the two α-helices in the full length SAMPs).

TABLE 1

| | | Full Length Amino Acid | Functional Fragment Amino |
|---|---|---|---|
| SAMP | Plant Source | Sequence | Acid Sequence |
| CghSAMPa; also | Microcitrus | MCCNRGKNVSIENLHQGFTH | HPSHVEYANLFLANLEKVLVI |
| referred to as | astralis × M. | IFESTFESTEGVAEYVSHPSH | DYKPTTVRV (SEQ ID NO: 17) |
| MaSSAMP and | australasica; | VEYANLFLANLEKVLVIDYK | |
| Syd72SSAMP1 | M. australasica | PTTVRV (SEQ ID NO: 1) | |
| | Eremocitrus | | |
| | glauca × citrus sp. | | |
| CghSAMPb; also | Eremocitrus | MCCNRGKNVSIENLHQGFPH | HPAHVEYANLFLANLEKVLVI |
| referred to | glauca × citrus | LFEFTFESTEGVAEYVSHPAH | DYKPTTVRV (SEQ ID NO: 18) |
| Syd72SSAMP2 | sp.; | VEYANLFLANLEKVLVIDYK | |
| | Microcitrus | PTTVRV (SEQ ID NO: 2) | |
| | australis × M. | | |
| | australasica; | | |
| | M. australasica | | |
| 942SAMP1, US-942; | Citrus reticulata × | MCCNRGKNVSIENLHQGFTH | HPAHVEYANLFLANLEKVLVI |
| also referred to | Poncirus trifoliata | IFESTFESTEGVAEYVAHPAH | DYKPTTERV (SEQ ID NO: 19) |
| 942SSAMP | | VEYANLFLANLEKVLVIDYK | |
| | | PTTERV (SEQ ID NO: 3) | |
| EgSSAMP1; also | Eremocitru s | MCCNRGKNVSIENLHQGFTH | HPAHVEYANLFLANLEKVLVI |
| referred to PtM_ | glauca; | IFESTFESTEGVAEYVAHPAH | DYKPTTVRV (SEQ ID NO: 18) |
| SSAMP, and | Poncirus | VEYANLFLANLEKVLVIDYK | |
| ClaSSAMP | trifoliata, ‘Monoe | PTTVRV (SEQ ID NO: 4) | |
| | mbryonic’; | | |
| | Citrus latipes | | |
| CsSAMP2, also | Citrus sinensis | MIAELIRSCCGLELLAVKYK | HPAHVEYANLFLANLEKVLVI |
| referred to | | GKNVSIENLHQGFTHIFESTF | DYKPTTVRV (SEQ ID NO: 18) |
| CsLSAMP2 | | ESTEGVAEYVAHPAHVEYA | |
| | | NLFLANLEKVLVIDYKPTTV | |
| | | RV (SEQ ID NO: 5) | |
| CsSAMP3, also | Citrus sinensis | MEEAKGVVKHVLLAKFKEG | HPAHVEYANLFLANLEKVLVI |
| referred to | | TAQDQIDQLIKDYANLVNLIE | DYKPTTVRV (SEQ ID NO: 18) |
| CsLSAMP3 | | PMKSFQWGKDVSIENRHQGF | |
| | | THIFESTFESTEGVAEYVAHP | |
| | | AHVEYANLFLANLEKVLVID | |
| | | YKPTTVRV (SEQ ID NO: 6) | |
| CsSAMP4, also | Citrus sinensis | MGEGEEAAMGEFKHLVIVKF | HPSHVEFSATFSAAIEKIVLLDF |
| referred to | | KEGVVVEDIVKGMKKLVSEI | PTVLGKAPAA (SEQ ID NO: 20) |
| CsLSAMP4 | | DAVKSFEWGQDVEGQEMLR | |
| | | QGFTHAFLMTFNKKEDYTTF | |
| | | ASHPSHVEFSATFSAAIEKIVL | |
| | | LDFPTVLGKAPAA (SEQ ID | |
| | | NO: 7) | |
| CcSAMP1, also | Citrus clementina | MKAETKGRDMEEAKGVVK | HPAHVEYANLFLANLEKVLVI |
| referred to | | HVLLAKFKEGTAQDQIDQLI | DYKPTTVRV (SEQ ID NO: 18) |
| CcLSAMP1 | | KDYANLVNLIEPMKSFQWG | |
| | | KDVSIENLHQGFTHIFESTFES | |
| | | TEGVAEYVAHPAHVEYANL | |
| | | FLANLEKVLVIDYKPTTVRV | |
| | | (SEQ ID NO: 8) | |
| CaSAMP, also | Capsicum annuum | MSYGRGKDVSTENLQQGFT | HPVHVEFANLMLPQLEKVLVI |
| referred to CaSSAMP | | HVFESTFDSTEGVAEYVSHP | DYKPEKVGP (SEQ ID NO: 21) |
| | | VHVEFANLMLPQLEKVLVID | |
| | | YKPEKVGP (SEQ ID NO: 9) | |
| NbSAMP, also | Nicotiana | MEGGKVKHILLAKFKDGIPA | HPAHVEYANTLLPQLEKVLVI |
| referred to NbLSAMP | benthamiana | DQIDQLIKQYANLVNLIEPM | DYKPEKVGP (SEQ ID NO: 22) |
| | | KAFHWGENVSIENFHQGFTH | |
| | | VFESTFDSTEGIAEYIDHPAH | |
| | | VEYANTLLPQLEKVLVIDYK | |
| | | PEKVGP (SEQ ID NO: 10) | |
| SISAMP, also referred | Solanum | MEGGKGGVVKHILLAKFKD | HPVHVEYANTLLPQLEKFLIV |
| to SILSAMP | lycopersicum | GIPPEQIDQLIKQYANLVNLV | DYKPQ (SEQ ID NO: 23) |
| | | EPMKAFQWGKDVSIENLHQ | |
| | | GFTHVFESTFDSLEGVAEYIA | |
| | | HPVHVEYANTLLPQLEKFLIV | |
| | | DYKPQ (SEQ ID NO: 11) | |
| SmSAMP, also | Solanum | MNIAVFLPSSCPALPRSKASR | HPAHVEYANILLTQLEKVLVID |
| referred to SmLSAMP | melongena | PSPPGQFPFLAKNVQLLLVLR | YKPEKLSP (SEQ ID NO: 24) |
| | | SYSSTARAMSLRGENVSIENL | |
| | | HQGFTHVFESTFDSVEGIAEY | |
| | | IDHPAHVEYANILLTQLEKVL | |
| | | VIDYKPEKLSP (SEQ ID | |
| | | NO: 12) | |
| StSAMP, also referred | Solanum | MEGGKGGVVKHILLAKFKD | HPVHVEFANTMLPQLEKVLIID |
| to StLSAMP | tuberosum | GIPPEQIDQLIKQYANLVNLIE | YKPQ (SEQ ID NO: 25) |
| | | PMKAFQWGKDVSIENLHQG | |
| | | FTHVFESTFDSLEGVAEYIAH | |
| | | PVHVEFANTMLPQLEKVLIID | |
| | | YKPQ (SEQ ID NO: 13) | |
| PtSAMP1 a, also | Ponirus trifoliate | MCCNRGKNVSIENLHQGFTH | HPAHVEYANSFLANLEKVLVI |
| referred to | ‘Flying Dragon’, | IFESTFESTEGVAEYVAHPAH | DYKPTTVRV (SEQ ID NO: 26) |
| PtSSAMPla | ‘Florida’, ‘Kryder | VEYANSFLANLEKVLVIDYK | |
| | 55-5’, | PTTVRV (SEQ ID NO: 14) | |
| | ‘Christiansen’, | | |
| | ‘Nanjing’ | | |
| PtSAMP1b, also | Poncirus trifoliate | MCCNRGKNVSIENLHQGFTH | HPAHVEYTNSFLANLEKVLVI |
| referred to | Flying Dragon’; | IFESTFESTEGVAEYVAHPAH | DYKPTTVRV (SEQ ID NO: 27) |
| PtSSAMP1b | ‘Florida’ | VEYTNSFLANLEKVL VIDYK | |
| | | PTTVRV (SEQ ID NO: 15) | |
| MCaSAMP; | Microcitrus | MCCNRGKNVSIENLHQGFTH | HPAHVEYANLFLANLEKVLVI |
| also referred to | australasica | IFESTFESTEGVAEYVSHPAH | DYKPTTVRV (SEQ ID NO: 18) |
| MaSSAMP2, | Eremocitrus | VEYANLFLANLEKVL VIDYK | |
| EgSSAMP2, | glauca; | PTTVRV (SEQ ID NO: 16) | |
| PtT SSAMP | Poncirus trifoliata, | | |
| | ‘Taxes' | | |
| MpSSAMP | Murraya | MCCNRGKNVSVENLHQGFT | HPVHLEFANLFLANLEKVLVI |
| | paniculata, | HIFESTFESTEGVAEYVAHPV | DYKPTTVRV (SEQ ID NO: 49) |
| | Hawaiian mock | HLEFANLFLANLEKVL VIDY | |
| | orange | KPTTVRV (SEQ ID NO: 48) | |

Table 2 provides the sequences of Functional SAMP helix fragments:

TABLE 2

| Functional SAMP helix Fragment | | |
|---|---|---|
| Amino Acid Sequence | From SSAMP | Plant Source |
| SHVEYANLFLANLEKVLVIDYK | CghSAMPa; also referred to | Microcitrus australis × M. |
| (SEQ ID NO: 34) | as MaSSAMP and | australasica; |
| | Syd72SSAMP1 | M. australasica |
| | | Eremocitrus glauca × citrus sp. |
| AHVEYANLFLANLEKVLVIDYK | CghSAMPb; also referred to | Eremocitrus glauca × citrus sp.; |
| (SEQ ID NO: 35) | Syd72SSAMP2 | |
| | EgSSAMP1; also referred to | |
| | PtM_SSAMP, and | Microcitrus australis × M. |
| | ClaSSAMP | australasica; |
| | MCaSAMP; | M. australasica |
| | also referred to | Eremocitrus |
| | MaSSAMP2, EgSSAMP2, | glauca; |
| | PtTSSAMP | Poncirus trifoliata, ‘Monoembryonic’; |
| | 942SAMP1, US-942; also | Citrus latipes |
| | referred to 942SSAMPI | Microcitrus australasica |
| | | Eremocitrus glauca; |
| | | Poncirus trifoliata, ‘Taxes’ |
| | | Citrus reticulata × Poncirus trifoliata |
| AHVEYANSFLANLEKVLVIDYK | PtSAMP1a, also referred to | Poncirus trifoliate |
| (SEQ ID NO: 43) | PtSSAMP1a | ‘Flying Dragon’, ‘Florida’, ‘Kryder 55- |
| | | 5’, ‘Christiansen’, ‘Nanjing’ |
| AHVEYTNSFLANLEKVLVIDYK | PtSAMP1b, also referred to | Poncirus trifoliate |
| SEQ ID NO: 44) | PtSSAMP1b | Flying Dragon’; ‘Florida’ |
| VHLEFANLFLANLEKVLVIDYK | MpSSAMP | Murraya paniculata, |
| (SEQ ID NO: 50) | | Hawaiian mock orange |
| VHVEFANLMLPQLEKVLVIDYK | CaSAMP, also referred to | Capsicum annuum |
| (SEQ ID NO: 38 | CaSSAMP | |
| AHVEYANLFLANLEKVLVIDYK | CILSAMP1 | Eremocitrus glauca × citrus sp.; |
| (SEQ ID NO: 35) | CILSAMP2 | Microcitrus australis × M. |
| (Identical to the helix from CghSAMPb, | MaLSAMP | australasica; |
| also referred to Syd72SSAMP2; | EgLSAMP1 | M. australasica |
| EgSSAMP1, also referred to PtM_ | EgLSAMP2 | Eremocitrus glauca; |
| SSAMP, and ClaSSAMP; MCaSAMP, | PtMLSAMP | Poncirus trifoliata, ‘Monoembryonic’; |
| also referred to MaSSAMP2, | ClaLSAMP | Citrus latipes |
| EgSSAMP2, PITSSAMP; 942SAMP1, | | Citrus sinensis |
| US-942; also referred to 942SSAMP1) | | Citrus clementina |
| | | Microcitrus australasica |
| SHVEFSATFSAAIEKIVLLDFPT | CsSAMP4, also referred to | Citrus sinensis |
| (SEQ ID NO: 51) | CsLSAMP4 | |
| AHVEYANTLLPQLEKVLVIDYK | NbSAMP, also referred to | Nicotiana benthamiana |
| (SEQ ID NO: 39) | NbLSAMP | |
| VHVEYANTLLPQLEKFLIVDYK | SISAMP, also referred to | Solanum lycopersicum |
| (SEQ ID NO: 40) | SILSAMP | |
| AHVEYANILLTQLEKVLVIDYK | SmSAMP, also referred to | Solanum melongena |
| (SEQ ID NO: 41) | SmLSAMP | |
| VHVEFANTMLPQLEKVLIIDYK | StSAMP, also referred to | Solanum tuberosum |
| (SEQ ID NO: 42) | SILSAMP | |
| AHVEYANSFLANLEKVLVIDYK | PtKLSAMP | Poncirus trifoliate |
| (SEQ ID NO: 43) | PtCLSAMP | ‘Kryder 55-5’, ‘Christiansen’, |
| AHVEYTNSFLANLEKVLVIDYK | PtFDLSAMP | Poncirus trifoliate |
| (SEQ ID NO: 44) | PtFLSAMP | ‘Flying Dragon’; ‘Florida’; ‘Taxes’ |
| | PtTLSAMP | |
| AHVEYANSFLANLDKVLVIDYK | PINLSAMP | Poncirus trifoliate |
| (SEQ ID NO: 52) | | ‘Nanjing’ |
| VHLEFANLFLANLEKVLVIDYK | MpLSAMPI | Murraya paniculata, |
| (SEQ ID NO: 50) | MpLSAMP2 | Hawaiian mock orange |
| (Identical to the helix from MpSSAMP) | | |

Further, SAMPs disclosed herein also include:

(SEQ ID NO: 28)
HPAHVEFATIFLGSLDKVLVIDYKPTSVSL
from Arabidopsis,
(SEQ ID NO: 29)
HPAHVEFANEFLPALEKTLIIDYKPTSGNNS
from Oryza brachyantha,
and
(SEQ ID NO: 30)
HPAHVEFANLFLSHVEKVIVFDYKPTTVRV
from Prunus mume.

The sequence alignment of SEQ ID NOS: 17-30 is shown below

| | | |
|---|---|---|
| SEQ17 | HPSHVEYANLFLANLEKVLVIDYKPTTVRV--- | 30 |
| SEQ18 | HPAHVEYANLFLANLEKVLVIDYKPTTVRV--- | 30 |
| SEQ19 | HPAHVEYANLFLANLEKVLVIDYKPTTERV--- | 30 |
| SEQ20 | HPSHVEFSATFSAAIEKIVLLDEPTVLGKAPAA | 33 |
| SEQ21 | HPVHVEFANLMLPQLEKVLVIDYKPEKVGP--- | 30 |
| SEQ22 | HPAHVEYANTLLPOLEKVLVIDYKPEKVGP--- | 30 |
| SEQ23 | HPVHVEYANTLLPQLEKFLIVDYKPQ------- | 26 |
| SEQ24 | HPARVEYANILLTQLEKVIVIDYKPEKLSP--- | 30 |
| SEQ25 | HPVHVEFANTMLPOLEKVLIIDYKPQ------- | 26 |
| SEQ26 | HPAHVEYANSFLANLEKVLVIDYKPTTVRV--- | 30 |
| SEQ27 | HPAHVEYINSFLANLEKVLVIDYKPTTVRV--- | 30 |
| SEQ28 | HPAHVEFATIFLGSLDKVLVIDYKPTSVSL--- | 30 |
| SEQ29 | HPAHVEFANEFLPALEKTLIIDYKPTSGNNS-- | 31 |
| SEQ30 | HPAHVEFANLFLSHVEKVIVEDYKPTTVRV--- | 30 |
| SEQ49 | HPVHLEFANLFLANLEKVLVIDYKPTTVRV | |

The present disclosure provides a polypeptide comprising a SAMP that comprises a sequence of: HPX₁H(V/L)EX₂X₃X₄X₅X₆X₇X₈X₉X₁₀X₁₁KX₁₂X₁₃X₁₄X₁₅DX₁₆X₁₇X₁₈X₁₉X₂₀X₂₁X₂₂X₂₃X₂₄X₂₅X₂₆ (SEQ ID NO:31), wherein X₁ is S, A, or V; X₂ is Y or F; X₃, is A, S, or T; X₄ is N, A, or T; X₅ is L, T, I, S, or E; X₆ is F, M, or L; X₇ is L or S; X₈ is A, P, T, G, or S; X₉ is N, A, Q, S, or H; X₁₀ is L, I, or V; X₁₁ is E or D; X₁₂ is V, I, F, or T; X₁₃ is L, V, or I; X₁₄ is V, L, or I; X₁₅ is I, L, V, or F; X₁₆ is Y or F; X₁₇ is K or P; X₁₈ is P or T; X₁₉ is T, V, E, or Q; X₂₀ is T, L, K, S, or absent; X₂₁ is V, E, G, L, or absent; X₂₂ is R, K, G, S, N, or absent; X₂₃ is V, A, P, L, N, or absent; X₂₄ is P, S, or absent; X₂₅ is A or absent; and X₂₆ is A or absent, and wherein the SAMP comprises a single α-helix structure. For example, a polypeptide can have a SAMP comprising a sequence of any one of SEQ ID NOS:17-27 and 49 listed in Table 1 and SEQ ID NOS:28-30.

The present disclosure further provides a SAMP comprising an α-helix and a sequence:

(SEQ ID NO: 53)
(V/S/A)H(V/L)E(F/Y)(A/T/S)(N/A/T)(L/E/I/S/T)(F/L/M)
(L/S)(A/S/P/G/T)(N/A/Q/S)(L/V/I)(E/D)K(V/I/T/F)
(L/I/V)(V/L/I)(I/L/V/F)DYK.

In some embodiments, the SAMP comprises

(SEQ ID NO: 54)
(V/S/A)H(V/L)E(F/Y)(A/T/S)N(L/E/I/S/T)(F/L/M)(L/S)
(A/S/P/G/T)(N/A/Q/S)(L/V/I)(E/D)K(V/I/T)(L/I/V)
(V/L/I)(I/L/V)DYK.

In some embodiments, the SAMP comprises

(SEQ ID NO: 55)
(V/S/A)HVE(F/Y)AN(L/I/S/T)(F/L)(L/S)(A/P/)(N/A/Q/S)
LEKV(L/I)(V/L/I)(I/L/V)DYK.

In some embodiments, the SAMP comprises

(SEQ ID NO: 56)
(V/S/A)HVE(F/Y)AN(L/I/S/T)(F/L)LA(N/Q)LEKV(L/I)
(V/L/I)(I/L/V)DYK.

In some embodiments, the SAMP comprises

(SEQ ID NO: 34)
SHVEYANLFLANLEKVIVIDYK;
(SEQ ID NO: 35)
AHVEYANLFLANLEKVLVIDYK;
(SEQ ID NO: 36)
AHVEYANLFLANLEKVIVIDYK;
(SEQ ID NO: 37)
SHVEFSATFSAAIEKIVILDFP;
(SEQ ID NO: 38)
VHVEFANLMLPQLEKVLVIDYK;
(SEQ ID NO: 39)
AHVEYANTLLPQLEKVIVIDYK;
(SEQ ID NO: 40)
VHVEYANTLLPQLEKFLIVDYK;
(SEQ ID NO: 41)
AHVEYANILLTQLEKVLVIDYK;
(SEQ ID NO: 42)
VHVEFANTMLPQLEKVLIIDYK;
(SEQ ID NO: 43)
AHVEYANSFLANLEKVLVIDYK;
(SEQ ID NO: 44)
AHVEYTNSFLANLEKVLVIDYK;
(SEQ ID NO: 45)
AHVEFATIFLGSLDKVIVIDYK;
(SEQ ID NO: 46)
AHVEFANEFLPALEKTLIIDYK;
(SEQ ID NO: 47)
AHVEFANLFLSHVEKVIVEDYK;
(SEQ ID NO: 50)
VHLEFANLFLANLEKVLVIDYK;
(SEQ ID NO: 51)
SHVEFSATFSAAIEKIVLLDFPT;
or
(SEQ ID NO: 52)
AHVEYANSFLANLDKVLVIDYK.

In some embodiments, the SAMP comprises an α-helix and has at least 70% sequence identity or at least 75% sequence identity to any one of SEQ ID NOS:34-47, 50, 51, and 52. In some embodiments, the SAMP comprises an α-helix and has at least 80% sequence identity to any one of SEQ ID NOS:34-47, 50, 51, and 52. In some embodiments, the SAMP comprises an α-helix and has at least 85% sequence identity to any one of SEQ ID NOS:34-47, 50, 51, and 52. In some embodiments, the SAMP comprises an α-helix and has at least 90% sequence identity to any one of SEQ ID NOS:34-47, 50, 51, and 52. In some embodiments, the SAMP comprises an α-helix and has at least 95% sequence identity to any one of SEQ ID NOS:34-47, 50, 51, and 52. In some embodiments, the SAMP comprises an amino acid sequence of any one of SEQ ID NOS:34-47, 50, 51, and 52.

In some embodiments, the SAMP has at least 70% sequence identity or at least 75% sequence identity to SEQ NO:34. In some embodiments, the SAMP has at least 800% sequence identity to SEQ NO:34. In some embodiments, the SAMP has at least 85% sequence identity to SEQ NO:34. In some embodiments, the SAMP has at least 90% sequence identity to SEQ NO:34. In some embodiments, the SAMP has at least 95% sequence identity to SEQ NO:34. In some embodiments, the SAMP comprises the amino acid sequence of SEQ ID NO:34.

In some embodiments, the SAMP comprises an α-helix and has no more than four substitutions relative to any one of SEQ ID NOS:34-47, 50, 51, and 52. In some embodiments, the SAMP comprises an α-helix and has no more than three substitutions relative to any one of SEQ ID NOS:34-47, 50, 51, and 52. In some embodiments, the SAMP comprises an α-helix and has no more than two substitutions relative to any one of SEQ ID NOS:34-47, 50, 51, and 52. In some embodiments, the SAMP comprises an α-helix and has no more than one substitution relative to any one of SEQ ID NOS:34-47, 50, 51, and 52.

In some embodiments, the SAMP comprises an α-helix and has no more than four substitutions relative to SEQ ID NO:34. In some embodiments, the SAMP comprises an α-helix and has no more than three substitutions relative to SEQ ID NO:34. In some embodiments, the SAMP comprises an α-helix and has no more than two substitutions relative to SEQ ID NO:34. In some embodiments, the SAMP comprises an α-helix and has no more than one substitution relative to SEQ ID NO:34. For example, in some embodiments, a hydrophobic residue of the α-helix may be substituted with another hydrophobic residue.

The present disclosure provides a polypeptide comprising a SAMP that comprises a sequence having at least 90% sequence identity (e.g., 90%, 91%, 92%, 93%, 94%, 95%, 96%, 97%, 98%, 99%, or 100% sequence identity) to a sequence of any one of SEQ ID NOS:17-27 and 49 listed in Table 1 and SEQ ID NOS:28-30, wherein the SAMP comprises a single α-helix structure. In some embodiments, the present disclosure provides a polypeptide comprising a SAMP that comprises a sequence having at least 80% sequence identity (e.g., 80%, 81%, 82%, 83%, 84%, 85%, 86%, 87%, 88%, 89%, 90%, 91%, 92%, 93%, 94%, 95%, 96%, 97%, 98%, 99%, or 100% sequence identity) to a sequence of any one of SEQ ID NOS:17-27 and 49 listed in Table 1 and SEQ ID NOS:28-30; or to a sequence of any one of SEQ ID NOS:34-47, 50, 51, and 52 listed in Table 2.

The present disclosure also provides a polypeptide comprising a SAMP that comprises a sequence having at least one amino acid substitution (e.g., 2, 3, 4, 5, 6, 7, 8, 9, 10, 11, 12, 13, 14, or 15 amino acid substitutions) relative to a sequence of any one of SEQ ID NOS:17-27 and 49 listed in Table 1 and SEQ ID NOS:28-30; or to a sequence of any one of SEQ ID NOS:34-47, 50, 51, and 52 listed in Table 2.

In certain embodiments, the disclosure also provides methods of producing a plant (e.g. a citrus plant) having enhanced Liberibacter disease resistance (e.g., HLB resistance) or Liberibacter disease tolerance (e.g., HLB tolerance) (i.e., enhanced resistance or tolerance to a bacterial infection caused by bacteria in the genus Liberibacter (e.g., Candidatus Liberibacter species) or Liberibacter crescens) by introducing a mutation into a polynucleotide in the plant, in which the mutated polynucleotide encodes a polypeptide comprising a SAMP of the present disclosure, such as the SAMPs having at least 900% sequence identity (e.g., 90%, 91%, 92%, 93%, 94% 95%, 96%, 97%, 98%, 99%, or 100% sequence identity) or at least one amino acid substitution (e.g., 2, 3, 4, 5, 6, 7, 8, 9, 10, 11, 12, 13, 14, or 15 amino acid substitutions) relative to the sequence of any one of SEQ ID NOS:17-30, and 49; or any one of SEQ ID NOS:34-47 and 50-52.

The disclosure also provides methods of preventing or treating a Liberibacter disease (e.g., HLB) and/or preventing or treating a bacterial infection caused by bacteria in the genus Liberibacter (e.g., Candidatus Liberibacter species or Liberibacter crescens) in a plant by contacting the plant with a polypeptide comprising a SAMP of the present disclosure (e.g., the SAMPs having at least 90% sequence identity (e.g., 90%, 91%, 92%, 93%, 94% 95%, 96%, 97%, 98%, 99%, or 100% sequence identity) or at least one amino acid substitution (e.g., 2, 3, 4, 5, 6, 7, 8, 9, 10, 11, 12, 13, 14, or 15 amino acid substitutions) relative to the sequence of any one of SEQ ID NOS:17-30, 49; or SEQ ID NOS: 34-47 and 50-52. Without being bound by any theory, SAMPs (e.g., HS peptides) can target and destroy bacterial cells, and/or induce defense response in plants, thus, enhancing the Liberibacter disease resistance or Liberibacter disease tolerance of plants. An example of a bacterial infection caused by bacteria in the genus Liberibacter (e.g., Candidatus Liberibacter species or Liberibacter crescens) that can be treated or prevented as described herein is potato zebra chip disease. For example, potato zebra disease can be treated or prevented in potato or tomato plants.

### III. Agricultural Compositions

The disclosure also provides agricultural compositions that contain one or more of the polypeptides described herein for use in preventing or treating a bacterial disease (e.g., a Liberibacter disease (HLB) or potato Zebra Chip disease, and other bacterial diseases such as those caused by Agrobacterium tumefaciens (also known as Rhizobium radiobacter) and Pseudomonas syringae) in a plant. The polypeptides described herein contain a SAMP that has only a single α-helix structure. A SAMP can have a sequence that has at least 90% sequence identity (e.g., 90%, 91%, 92%, 93%, 94% 95%, 96%, 97%, 98%, 99%, or 100% sequence identity) or at least one amino acid substitution (e.g., 2, 3, 4, 5, 6, 7, 8, 9, 10, 11, 12, 13, 14, or 15 amino acid substitutions) relative to the sequence of any one of SEQ ID NOS:17-30, and 49; or any one of SEQ ID NOS:34-47, 50, 51, and 52). A SAMP can also have a sequence of SEQ ID NO:31. In some embodiments, the agricultural composition further includes at least one of an herbicide, an herbicide safener, a surfactant, a fungicide, a pesticide, a nematicide, a plant activator, a synergist, a plant growth regulator, an insect repellant, an acaricide, a molluscicide, or a fertilizer.

An agricultural composition comprising one or more polypeptides described herein can also include one or more of: a surface-active agent, an inert carrier, a preservative, a humectant, a feeding stimulant, an attractant, an encapsulating agent, a binder, an emulsifier, a dye, a UV protective, a buffer, a flow agent, a fertilizer, a nitrogen fixation agent, micronutrient donors, or other preparations that influence plant growth. The agricultural composition can also include one or more agrochemicals including: herbicides, insecticides, fungicides, bactericides, nematicides, molluscicides, acaracides, plant growth regulators, harvest aids, and fertilizers, which can also be combined with carriers, surfactants or adjuvants as appropriate for the agrochemical. Suitable carriers and adjuvants can be solid or liquid and correspond to the substances ordinarily employed in formulation technology, e.g., natural or regenerated mineral substances, solvents, dispersants, wetting agents, tackifiers, binders, or fertilizers. The active ingredients of the present disclosure are normally applied in the form of compositions and can be applied to the crop area, plant, or seed to be treated. For example, the compositions of the present disclosure can be applied during growth, seeding, or storage.

Surface-active agents that can be used with the presently described polypeptides include anionic compounds such as a carboxylate of, for example, a metal; carboxylate of a long chain fatty acid; an N-acylsarcosinate; mono- or di-esters of phosphoric acid with fatty alcohol ethoxylates or salts of such esters; fatty alcohol sulfates such as sodium dodecyl sulfate, sodium octadecyl sulfate or sodium cetyl sulfate; ethoxylated fatty alcohol sulfates; ethoxylated alkylphenol sulfates; lignin sulfonates; petroleum sulfonates; alkyl aryl sulfonates such as alkyl-benzene sulfonates or lower alkylnaphtalene sulfonates, e.g., butyl-naphthalene sulfonate; salts of sulfonated naphthalene-formaldehyde condensates; salts of sulfonated phenol-formaldehyde condensates; more complex sulfonates such as the amide sulfonates, e.g., the sulfonated condensation product of oleic acid and N-methyl taurine; or the dialkyl sulfosuccinates, e.g., the sodium sulfonate or dioctyl succinate. Non-ionic agents include condensation products of fatty acid esters, fatty alcohols, fatty acid amides or fatty-alkyl- or alkenyl-substituted phenols with ethylene oxide, fatty esters of polyhydric alcohol ethers, e.g., sorbitan fatty acid esters, condensation products of such esters with ethylene oxide, e.g., polyoxyethylene sorbitar fatty acid esters, block copolymers of ethylene oxide and propylene oxide, acetylenic glycols such as 2,4,7,9-tetraethyl-5-decyn-4,7-diol, or ethoxylated acetylenic glycols. Examples of a cationic surface-active agent include, for instance, an aliphatic mono-, di-, or polyamine such as an acetate, naphthenate or oleate; or oxygen-containing amine such as an amine oxide of polyoxyethylene alkylamine; an amide-linked amine prepared by the condensation of a carboxylic acid with a di- or polyamine; or a quaternary ammonium salt.

Examples of inert materials or inert carriers that can be used include, but are not limited to, inorganic minerals such as kaolin, phyllosilicates, carbonates, sulfates, phosphates, or botanical materials such as cork, powdered comcobs, peanut hulls, rice hulls, and walnut shells.

Herbicides that can be used with the presently described polypeptides include compounds that kill or inhibit growth or replication of undesired plants, typically a subset of plants that is distinct from the desired plant or crop. There are several modes of action: ACCase inhibition, carotenoid biosynthesis inhibition, cell wall synthesis inhibition, ALS inhibition, ESP synthase inhibition, glutamine synthase inhibition, HPPD inhibition, microtubule assembly inhibition, PPO inhibition, etc. Examples of commercially available herbicides include One-Time®, MSMA, Corvus®, Volunteer®, Escalade®, Q4®, Raptor®, Acumen®, Sencor®, Bullet®, TopNotch®, Valor®, PastureGard®, glycophosate (Roundup®), DSMA, Break-Up®, Hyvar®, Barricade®, etc. Herbicides can be mixed with “herbicide safeners” to reduce general toxicity of the herbicide, as described, e.g., in Riechers et al. (2010) Plant Physiol. 153:3.

Pesticides (e.g., nematicides, molluscicides, insecticides, miticide/acaricides) can be used in combination with the presently disclosed polypeptides to kill or reduce the population of undesirable pests affecting the plant. Pesticides can also be used with repellants or pheromones to disrupt mating behavior. Insectides are directed to insects, and include, e.g., those of botanical origin (e.g., allicin, nicotine, oxymatrine, jasmolin I and II, quassia, rhodojaponin III, and limonene), carbamate insecticides (e.g., carbaryl, carbofuran, carbosulfan, oxamyl, nitrilacarb, CPMC, EMPC, fenobucarb), fluorine insecticides, formamidine insecticides, fumigants (e.g., ethylene oxide, methyl bromide, carbon disulfide), chitin synthesis inhibitors, macrocyclic lactone insecticides, neonicotinoid insecticides, organophosphate insectides, urea and thiourea insectides, etc. Nematicides affect nematodes, and include, e.g., organophosphorus nematicides (e.g., diamidafos, fosthiazate, heterophos, phsphamidon, triazophos), fumigant nematicides (e.g., carbon disulfide, methyl bromide, methyl iodide), abamectin, carvacrol, carbamate nematicides (e.g., benomvl, oxaml), etc. Molluscicides are directed to slugs and snails, and include, e.g., allicin, bromoacetamide, thiocarb, trifenmorph, fentin, copper sulfate, etc. Many pesticides target more than one type of pest, so that one or two can be selected to target insects, mollusks, nematodes, mitogens, etc.

Fertilizers typically provide macro- and micronutrients in a form that they can be utilized by the plant, or a plant-associated organism. These include, e.g., nitrogen, phosphorus, potassium, sulfur, calcium, potassium, boron, chlorine, copper, iron, manganese, molybdenum, zinc, nickel, and selenium. Fertilizers are often tailored to specific soil conditions or for particular crops or plants. Fertilizers that can be used include naturally-occurring, modified, concentrated and/or chemically synthesized materials, e.g., manure, bone meal, compost, fish meal, wood chips, etc., or can be chemically synthesized. UAN, anhydrous ammonium nitrate, urea, potash, etc. Suppliers include Scott®, SureCrop®, BCF®, RVR®, Gardenline®, and many others known in the art.

Fungicides are compounds that can kill fungi or inhibit fungal growth or replication. Fungicides that can be used with the presently disclosed polypeptides include contact, translaminar, and systemic fungicides. Examples include sulfur, neem oil, rosemary oil, jojoba, tea tree oil, Bacillus subtilis, Ulocladium, cinnamaldehyde, etc.

The agricultural compositions of the disclosure can be in a suitable form for direct application or as a concentrate of primary composition that requires dilution with a suitable quantity of water or other diluent before application. The concentration of a polypeptide containing a SAMP (e.g., a SAMP having a sequence of any one of SEQ ID NOS:17-32, 49; or a SAMP having a sequence of any one of SEQ ID NOS:34-47, 50, 51, and 52) in the agricultural composition will vary depending upon the nature of the particular formulation, specifically, whether it is a concentrate or to be used directly, the type of plant, and in some cases, on the nature of the use, e.g., for preventing a plant that is at risk of a Liberibacter disease (e.g., HLB) or for treating a plant that is already infected with a Liberibacter disease (e.g., HLB).

### IV. Methods of Preventing or Treating a Bacterial Disease

The polypeptides described herein can be used to prevent or treat a bacterial disease, e.g., a Gram-negative bacterial disease. A Liberibacter disease refers to an infection caused by Gram-negative bacteria in the genus Liberibacter (e.g., Candidatus Liberibacter species or Liberibacter crescens). A Liberibacter disease can infect plants such as citrus plants (e.g., orange, grapefruit, tangerine, lemon, line, key line, papeda, citron, and pomelo) and solanaceous plants (e.g., potato, tomato, eggplant, and pepper). Huanglongbing (HLB) is a type of Liberibacter disease that infects citrus plants. Potato Zebra Chip (ZC) disease is a type of Liberibacter disease that infects potato plants. The infection is vectored and transmitted by potato psyllids (e.g., Bactericera cockerelli). The methods of utilizing the polypeptides disclosed herein can also be used to prevent or treat other bacterial diseases (e.g., other Gram-negative bacterial diseases), such as those caused by Agrobacterium tumefaciens (also known as Rhizobium radiobacter) and Pseudomonas syringae.

HLB

The present disclosure also provides methods of preventing or treating HLB in plants. In some embodiments of the methods, the plants with HLB can be contacted with one or more polypeptides containing a SAMP (e.g., a SAMP having a sequence of any one of SEQ ID NOS:17-32, 49, and 34-47, and 50) or an agricultural composition comprising the one or more polypeptides described herein. In some embodiments, the polypeptides or agricultural composition can be injected into the trunk of the plant. In other embodiments, the polypeptide or agricultural composition can be injected into the stem of the plant. In yet other embodiments, the polypeptide or agricultural composition can be foliar sprayed onto the plant. In yet other embodiments, the polypeptide or agricultural composition, e.g., comprising a surfactant, can be applied by dripping irrigation to the plant. Once the plants are contacted with the polypeptides described herein, the polypeptides can enhance HLB resistance or HLB tolerance of the plants, thus, preventing or treating HLB in the plants.

The methods described herein can be used to reduce symptoms caused by HLB, including yellowing of leaves, blotchy mottle of the leaves, zinc-deficiency-like mottle, severe chlorosis, and reduced fruit yield. It will be understood that symptoms of HLB vary according to the time of infection, stage of the disease, tree species, and tree maturity, among other things. It will be further understood that in some embodiments, the disclosed methods may not necessarily result in eradication or cure of the infection but can significantly reduce the symptoms caused by HLB.

Thus, in some embodiments, the methods provided herein reduce the symptoms of HLB by reducing the yellowing of leaves, resulting in a greener appearance, increasing the growth rate of the plant, and/or increasing the fruit yield of the plant. Thus, in some embodiments, the fruit yield is improved by 5%, 10%, 20%, 30%, 40%, 50%, 60% 70%, 80%, 90%, 1000%, 200%, 500% or more compared to a plant that is not treated according to the methods. In some embodiments, the fruit yield is increased to 10%, 20%, 30%, 40%, 50%, 60%, 70%, 80%, 90%, or 100% of the yield of a similar plant that was not infected by HLB.

The methods described herein can also be used to prevent HLB-infection in plants. For example, a plant that is not yet infected with HLB, but is at risk for infection (i.e., the plant is located in an area where HLB is identified in surrounding plants) can be contacted by one or more SAMPs (e.g., HS peptides) as described in the methods of the disclosure. The plant at risk for HLB can also be genetically modified to express one or more polypeptides described herein to prevent HLB.

Potato ZC Disease

The present disclosure also provides methods of preventing or treating potato ZC disease in potato plants. In some embodiments of the methods, the plants with potato ZC disease can be contacted with one or more polypeptides containing a SAMP (e.g., a SAMP having a sequence of any one of SEQ ID NOS:17-32, 49, or a SAMP having a sequence of any one of SEQ ID NOS:34-47, 50, 51, and 52) or an agricultural composition comprising the one or more polypeptides described herein. In some embodiments, the polypeptide or agricultural composition can be injected into the tuber of the plant. In other embodiments, the polypeptide or agricultural composition can be applied to the roots of the plants. In yet other embodiments, the polypeptide or agricultural composition can be foliar sprayed onto the plant. Once the plants are contacted with the polypeptides described herein, the polypeptides can enhance potato ZC disease resistance or potato ZC disease tolerance of the plants, thus, preventing or treating potato ZC disease in the plants.

The methods described herein can be used to reduce symptoms caused by potato ZC disease, including chlorosis, leaf scorching, swollen nodes, vascular tissue browning, curled leaves, collapsed stolons, enlarged lenticels, vascular tissue browning, medullary ray discoloration, and necrotic flecking of tuber tissue. It will be understood that symptoms of potato ZC disease vary according to the time of infection, stage of the disease, plant species, and maturity, among other things. It will be further understood that in some embodiments, the disclosed methods can not necessarily result in eradication or cure of the infection, but can significantly reduce the symptoms caused by potato ZC disease.

Thus, in some embodiments, the methods provided herein reduce the symptoms of potato ZC disease as described above, resulting in a more healthy appearance, increasing the growth rate of the plant, and/or increasing the yield of the plant. Thus, in some embodiments, the yield is improved by 5%, 10%, 20%, 30%, 40%, 50%, 60% 70%, 80%, 90%, 100%, 200%, 500% or more compared to a plant that is not treated according to the methods. In some embodiments, the yield is increased to 10%, 20%, 300%, 400%, 50%, 60%, 70%, 80%, 90%, or 100% of the yield of a similar plant that was not infected by potato ZC disease.

The methods described herein can also be used to prevent potato ZC disease-infection in plants. For example, a plant that is not yet infected with potato ZC disease, but is at risk for infection (i.e., the plant is located in an area where potato ZC disease is identified in surrounding plants) can be contacted by one or more polypeptides as described in the methods of the disclosure. The plant at risk for potato ZC disease can also be genetically modified to express one or more polypeptides described herein to prevent potato ZC disease.

### V. Production of Plants Comprising the Polypeptides

In another aspect, the present disclosure provides for transgenic plants comprising recombinant expression cassettes for expressing a polypeptide containing a SAMP (e.g., a SAMP having a sequence of any one of SEQ ID NOS:17-32, 49, or of any one of SEQ ID NOS:34-47, 50, 51, and 52) as described herein in a plant. In some embodiments, a transgenic plant is generated that contains a complete or partial sequence of a polynucleotide that is derived from a species other than the species of the transgenic plant. It should be recognized that transgenic plants encompass the plant or plant cell in which the expression cassette is introduced as well as progeny of such plants or plant cells that contain the expression cassette, including the progeny that have the expression cassette stably integrated in a chromosome.

A recombinant expression vector comprising a polypeptide coding sequence driven by a heterologous promoter can be introduced into the genome of the desired plant host by a variety of conventional techniques. For example, the DNA construct can be introduced directly into the genomic DNA of the plant cell using techniques such as electroporation and microinjection of plant cell protoplasts, or the DNA construct can be introduced directly to plant tissue using ballistic methods, such as DNA particle bombardment. An exemplary vector is a viral vector that can express and optionally replicate in the plant. Exemplary viral vectors can include, for example, citrus tristeza virus (CTV) for expressing the peptide in a phloem-limited manner in citrus, or tobacco rattle virus (TRV) to express the antimicrobial peptides in potato or other plants. Alternatively, the DNA construct can be combined with suitable T-DNA flanking regions and introduced into a conventional Agrobacterium tumefaciens host vector. The virulence functions of the Agrobacterium tumefaciens host will direct the insertion of the construct and adjacent marker into the plant cell DNA when the cell is infected by the bacteria. While transient expression of the constitutively active polypeptide is encompassed by the disclosure, generally, expression of a construct of the disclosure will be from insertion of expression cassettes into the plant genome, e.g., such that at least some plant offspring also contain the integrated expression cassette.

Microinjection techniques are also useful for this purpose. These techniques are well known in the art and thoroughly described in the literature. The introduction of DNA constructs using polyethylene glycol precipitation is described in Paszkowski et al. EMBO J. 3:2717-2722 (1984). Electroporation techniques are described in Fromm et al. Proc. Natl. Acad. Sci. USA 82:5824 (1985). Ballistic transformation techniques are described in Klein et al. Nature 327:70-73 (1987).

Agrobacterium tumefaciens-mediated transformation techniques, including disarming and use of binary vectors, are well described in the scientific literature. See, for example, Horsch et al. Science 233:496-498 (1984), and Fraley et al. Proc. Natl. Acad. Sci. USA 80:4803 (1983).

Transformed plant cells derived by any of the above transformation techniques can be cultured to regenerate a whole plant that possesses the transformed genotype and thus the desired phenotype, e.g., resistance or tolerance to a Liberibacter disease (e.g., HLB). Such regeneration techniques rely on manipulation of certain phytohormones in a tissue culture growth medium, typically relying on a biocide and/or herbicide marker which has been introduced together with the desired nucleotide sequences. Plant regeneration from cultured protoplasts is described in Evans et al., Protoplasts Isolation and Culture, Handbook of Plant Cell Culture, pp. 124-176, MacMillilan Publishing Company, New York, 1983; and Binding, Regeneration of Plants, Plant Protoplasts, pp. 21-73, CRC Press, Boca Raton, 1985. Regeneration can also be obtained from plant callus, explants, organs, or parts thereof. Such regeneration techniques are described generally in Klee et al. Ann. Rev. of Plant Phys. 38:467-486 (1987).

One of skill in the art will recognize that after the expression cassette is stably incorporated in transgenic plants and confirmed to be operable, it can be introduced into other plants by sexual crossing. Any of a number of standard breeding techniques can be used, depending upon the species to be crossed.

The expression cassettes and other constructs of the disclosure can be used to confer Liberibacter disease resistance or tolerance on essentially any plant. Thus, the disclosure has use over a broad range of plants, as well as rootstocks, including species from the genus Citrus (e.g., Citrus maxima, Citrus medica, Citrus micrantha, Citrus reticulate, Citrus aurantilfolia, Citrus aurantium, Citrus latifolia, Citrus limon, Citrus limonia, Citrus paradise, Citrus sinensis, and Citrus tangerine) or species from the family Solanaceae (e.g., Solanum spp., Capsicum spp., and Nicotiana spp.). Species from the genus Solanum include, e.g., Solanum tuberosum, Solanum lycopersicum, Solanum melongena, Solanum aviculare, Solanum capsicasrum, Solanum crispum, Solanum laciniatum, Solanum laxum, Solanum pseudocapsicum, Solanum rantonneti, Solanum seaforthianum, and Solanum wendlandii. Species from the genus Capsicum include, e.g., Capsicum annuum, Capsicum baccatum, Capsicum campylopodium, Capsicum cardenasii, Capsicum chacoense, Capsicum cornutum, Capsicum dusenii, Capsicum eximiwn, Capsicum friburgense, Capsicum frutescens, Capsicum geminoihum, Capsicum havanense, Capsicum lanceolatum, Capsicum lycianthoides, Capsicum minutiflorum, Capsicum mositicum, Capsicum pubescens, Capsicum recurvatum, Capsicum schottianum, Capsicum spina-alba, Capsicum tovarii, and Capsicum villosum. Species from the genus Nicotiana include, e.g., Nicotiana acuminate, Nicotiana benthamiana, Nicotiana glauca, Nicotiana longiflora, Nicotiana rustica, Nicotiana tabacum, and Nicotiana occidentalis.

In particular embodiments, the plant is selected from the group consisting of Citrus reticulata, Citrus sinensis, Citrus clementina, Capsicum annuum, Solanum tuberosum, Solanum lycopersicum, Solanum melongena, and Nitotiana benthamiana. In particular embodiments, the plant is a sweet orange plant (Citrus sinensis). In particular embodiments, the plant is a clementine plant (Citrus clementina). In particular embodiments, the plant is a potato plant (Solanum tuberosum). In some embodiment, the plant is a vegetable- or fruit-producing plant.

Those of skill will recognize that a number of plant species can be used as models to predict the phenotypic effects of transgene expression in other plants. For example, it is well recognized that both tobacco (Nicotiana) and Arabidopsis plants are useful models of transgene expression, particularly in other dicots.

In some embodiments, the plants of the disclosure have enhanced mediated phenotypes mediated by one or more polypeptides described herein, for example enhanced bacterial disease (e.g., a Liberibacter disease (e.g., HLB and ZC) and other bacterial diseases such as those caused by Agrobacterium tumefaciens (also known as Rhizobium radiobacter) and Pseudomonas syringae) resistance or tolerance, as compared to plants that are otherwise identical except for expression of the polypeptide.

CRISPR/Cas

Plant gene manipulations can now be precisely tailored in non-transgenic organisms using the CRISPR/Cas9 genome editing method. In this bacterial antiviral and transcriptional regulatory system, a complex of two small RNAs—the CRISPR-RNA (crRNA) and the trans-activating crRNA (tracrRNA)—directs the nuclease (Cas9) to a specific DNA sequence complementary to the crRNA (Jinek, M., et al. Science 337, 816-821 (2012)). Binding of these RNAs to Cas9 involves specific sequences and secondary structures in the RNA. The two RNA components can be simplified into a single element, the single guide-RNA (sgRNA), which is transcribed from a cassette containing a target sequence defined by the user (Jinek, M., et al. Science 337, 816-821 (2012)). This system has been used for genome editing in humans, zebrafish, Drosophila, mice, nematodes, bacteria, yeast, and plants (Hsu, P. D., et al., Cell 157, 1262-1278 (2014)). In this system the nuclease creates double stranded breaks at the target region programmed by the sgRNA. These can be repaired by non-homologous recombination, which often yields inactivating mutations. The breaks can also be repaired by homologous recombination, which enables the system to be used for gene targeted gene replacement (Li, J.-F., et al. Nat. Biotechnol. 31, 688-691, 2013; Shan, Q., et al. Nat. Biotechnol. 31, 686-688, 2013). In some embodiments of the methods in the present disclosure, a gene encoding a wild-type or endogenous SAMP in a plant can be modified using the CAS9/CRISPR system to match the polynucleotide sequence encoding a SAMP described herein (e.g., a polynucleotide sequence encoding the SAMP having at least 80% sequence identity, or at least 90% sequence identity, or at least one amino acid substitution relative to the sequence of any one of SEQ ID NOS:17-30, 49; or any one of SEQ ID NOS:34-47, 50, 51, and 52). In some embodiments, a gene encoding a wild-type or endogenous SAMP in a plant can be modified using the CAS9/CRISPR system to match the polynucleotide sequence of any one of SEQ ID NOS:17-30, and 49; or any one of SEQ ID NOS:34-47, 50, 51, and 52, or a fragment thereof.

Accordingly, in some embodiments, instead of generating a transgenic plant, a native SAMP coding sequence in a plant or plant cell can be altered in situ to generate a plant or plant cell carrying a polynucleotide encoding a SAMP described herein (e.g., a SAMP having at least 90% sequence identity or at least one amino acid substitution relative to the sequence of any one of SEQ ID NOS:17-30, 49, or any one of SEQ ID NOS:34-47, 50, 51, and 52). For example, in some embodiments, CRISPR technology is used to introduce one or more nucleotide changes into a SAMP coding sequence in situ to change the appropriate codon to make a change corresponding to positions X₁ to X₂₆ as set forth in the sequence of SEQ ID NO:31. The CRISPR/Cas system has been modified for use in prokaryotic and eukaryotic systems for genome editing and transcriptional regulation. The “CRISPR/Cas” system refers to a widespread class of bacterial systems for defense against foreign nucleic acid. CRISPR/Cas systems are found in a wide range of eubacterial and archaeal organisms. CRISPR/Cas systems include type I, II, and III sub-types. Wild-type type II CRISPR/Cas systems utilize the RNA-mediated nuclease, Cas9 in complex with guide and activating RNA to recognize and cleave foreign nucleic acid. Cas9 homologs are found in a wide variety of eubacteria, including, but not limited to bacteria of the following taxonomic groups: Actinobacteria, Aquificae, Bacteroidetes-Chlorobi, Chlamydiae-Verrucomicrobia, Chlroflexi, Cyanobacteria, Firmicutes, Proteobacteria, Spirochaetes, and Thermotogae. An exemplary Cas9 protein is the Streptococcus pyogenes Cas9 protein. Additional Cas9 proteins and homologs thereof are described in, e.g., Chylinksi, et al., RNA Biol. 2013 May 1; 10(5): 726-737; Nat. Rev. Microbiol. 2011 June; 9(6): 467-477; Hou, et al., Proc Natl Acad Sci USA. 2013 Sep. 24:110(39):15644-9; Sampson et al., Nature. 2013 May 9; 497(7448):254-7; and Jinek, et al., Science. 2012 Aug. 17:337(6096):816-21.

Accordingly, in one aspect, a method is provided of using CRISPR/CAS9 to introduce at least one mutation into a plant cell is performed. In some embodiments, a method of altering a (e.g., native) nucleic acid encoding SAMP in a plant is provided. In some embodiments, the method comprises introducing into the plant cell containing and expressing a DNA molecule having a target nucleic acid encoding SAMP an engineered, non-naturally occurring Clustered Regularly Interspaced Short Palindromic Repeats (CRISPR)—CRISPR associated (Cas) (CRISPR-Cas) system. In some embodiments, the CRISPR-Cas system comprises one or more vectors comprising: a) a first regulatory element operable in a plant cell operably linked to at least one nucleotide sequence encoding a CRISPR-Cas system guide RNA that hybridizes with the target sequence, and b) a second regulatory element operable in a plant cell operably linked to a nucleotide sequence encoding a Type-II Cas9 protein, wherein components (a) and (b) are located on same or different vectors of the system, whereby the guide RNA targets the target sequence and the Cas9 protein cleaves the DNA molecule, whereby at least one mutation is introduced into the target nucleic acid encoding the SAMP, i.e., one or more mutations are introduced into the target nucleic acid encoding the SAMPs to alter the sequence of the target nucleic acid to match the polynucleotide sequence encoding an antimicrobial (e.g., antibacterial) peptide having the sequence of any one of SEQ ID NOS:17-32, 49, and 34-47, and 50. In some embodiments, the SAMP is selected from any of SEQ ID NOS:17-30, 49 and 3447, and 50, or a substantially identical polypeptide or fragment thereof.

In some embodiments, methods of using CRISPR/CAS9 technology to introduce at least one mutation into a (e.g., native) nucleic acid encoding SAMP can be applied to a broad range of plants, including species from the genus Citrus (e.g., Citrus maxima, Citrus medica, Citrus micrantha, Citrus reticulate, Citrus aurantiifolia, Citrus aurantium, Citrus latifolia, Citrus limon, Citrus limonia, Citrus paradise, Citrus sinensis, and Citrus tangerine) or species from the family Solanaceae (e.g., Solanum spp., Capsicum spp., and Nicotiana spp.). Species from the genus Solanum include, e.g., Solanum tuberosum, Solanum lycopersicum, Solanum melongena, Solanum aviculare, Solanum capsicastrum, Solanum crispum, Solanum laciniatum, Solanum laxum, Solanum pseudocapsicum, Solanum rantonneiii, Solanum seaforthianum, and Solanum wendlandii. Species from the genus Capsicum include, e.g., Capsicum annuum, Capsicwn baccatum, Capsicum campylopodium, Capsicum cardenasii, Capsicum chacoense, Capsicum cornutum, Capsicum dusenii, Capsicum eximium, Capsicum friburgense, Capsicum frutescens, Capsicum geminmfolium, Capsicum havanense, Capsicum lanceolatum, Capsicum lycianthoides, Capsicum minutifolium, Capsicum mositicum, Capsicum pubescens, Capsicum recurvatum, Capsicum schottianum, Capsicum spina-alba, Capsicum tovarii, and Capsicum villosum. Species from the genus Nicotiana include, e.g., Nicotiana acuminate, Nicotiana benthamiana, Nicotiana glauca, Nicotiana longiflora, Nicotiana rustica, Nicotiana tabacum, and Nicotiana occidentalis. In particular embodiments, the plant is selected from the group consisting of Citrus reticulata, Citrus sinensis, Citrus clementina, Capsicum annuum, Solanum tuberosum, Solanum hcoperswcum, Solanum melongena, and Nitotiana benthamiana. In particular embodiments, the plant is a sweet orange plant (Citrus sinensis). In particular embodiments, the plant is a clementine plant (Citrus clementina). In particular embodiments, the plant is a potato plant (Solanum tuberosum). In some embodiment, the plant is a vegetable- or fruit-producing plant.

In some embodiments, the mutation(s) introduced to the target nucleic acid sequence change the appropriate codon in the sequence to make change(s) corresponding to positions X₁ to X₂₆ as set forth in the sequence of SEQ ID NO:31. For example, after introducing the mutations to the target nucleic acid to change the appropriate codons, the modified nucleic acid sequence encode, at its corresponding positions, one or more amino acids as set forth in positions X₁ to X₂₆ of SEQ ID NO:31. Also provided as a plant or plant cell resulting from the above-described method. Such a plant will contain a non-naturally-occurring nucleic acid sequence encoding the SAMP.

### VI. Expression Cassettes

In some embodiments, the present disclosure provides expression cassettes comprising a polynucleotide encoding a polypeptide containing a SAMP (e.g., a SAMP having a sequence of any one of SEQ ID NOS:17-32, 49, or any one of SEQ ID NOS:34-47, 50, 51, and 52) of the disclosure, wherein introduction of the expression cassette into a plant results in a transgenic plant expressing the polypeptide. In some embodiments, a promoter can be operably linked to the polynucleotide encoding the polypeptide. The promoter can be heterologous to the polynucleotide. In some embodiments, the promoter can be inducible. In some embodiments, the promoter can plant tissue-specific (e.g., phloem-specific, tuber-specific, root-specific, stem-specific, trunk-specific, or leaf-specific).

Any of a number of means well known in the art can be used to drive polypeptide expression in plants. Any organ can be targeted, such as shoot vegetative organs/structures (e.g. leaves, stems, and tubers), roots, flowers and floral organs/structures (e.g. bracts, sepals, petals, stamens, carpels, anthers and ovules), seed (including embryo, endosperm, and seed coat) and fruit. Alternatively, the polynucleotide encoding a polypeptide described herein can be expressed specifically in certain cell and/or tissue types within one or more organs (e.g., guard cells in leaves using a guard cell-specific promoter). Alternatively, the polynucleotide encoding a polypeptide described herein can be expressed constitutively (e.g., using the CaMV 35S promoter).

To use a polynucleotide encoding a polypeptide described herein in the above techniques, recombinant DNA vectors suitable for transformation of plant cells are prepared. Techniques for transforming a wide variety of higher plant species are well known and described in the technical and scientific literature. See, e.g., Weising et a&. Ann. Rev. Genet. 22:421-477 (1988). A DNA sequence coding for the polypeptide preferably will be combined with transcriptional and translational initiation regulatory sequences which will direct the transcription of the sequence from the gene in the intended tissues of the transformed plant.

For example, a plant promoter fragment can be employed to direct expression of the polypeptide in all tissues of a transgenic plant. Such promoters are referred to herein as “constitutive” promoters and are active under most environmental conditions and states of development or cell differentiation. Examples of constitutive promoters include the cauliflower mosaic virus (CaMV) 35S transcription initiation region, the 1′- or 2′-promoter derived from T-DNA of Agrobacterium tumafaciens, and other transcription initiation regions from various plant genes known to those of skill.

Alternatively, the plant promoter can direct expression of the polypeptide in a specific tissue (tissue-specific promoters) or can be otherwise under more precise environmental control (inducible promoters). Examples of tissue-specific promoters under developmental control include promoters that initiate transcription only in certain tissues, such as phloem, tubers, stems, trunks, leaves, or guard cells. Examples of environmental conditions that can affect transcription by inducible promoters include, but are not limited to, anaerobic conditions, elevated temperature, and the presence of light.

In some embodiments, a polyadenylation region at the 3-end of the coding region of the polypeptide can be included. The polyadenylation region can be derived from a naturally occurring SAMP gene, from a variety of other plant genes, or from T-DNA.

The vector comprising the polynucleotide sequences (e.g., promoters or polypeptide coding regions) can include a marker gene that confers a selectable phenotype on plant cells. For example, the marker can encode biocide resistance, particularly antibiotic resistance, such as resistance to kanamycin, G418, bleomycin, hygromycin, or herbicide resistance, such as resistance to chlorosluforon or Basta.

In some embodiments, the polynucleotide encoding the polypeptide is expressed recombinantly in plant cells. A variety of different expression constructs, such as expression cassettes and vectors suitable for transformation of plant cells, can be prepared. Techniques for transforming a wide variety of higher plant species are well known and described in the technical and scientific literature. See, e.g., Weising et al. Ann. Rev. Genet. 22:421-477 (1988). A DNA sequence coding for a polypeptide described herein can be combined with cis-acting (promoter) and trans-acting (enhancer) transcriptional regulatory sequences to direct the timing, tissue type, and levels of transcription in the intended tissues of the transformed plant. Translational control elements can also be used.

Embodiments of the present disclosure also provide for a polynucleotide encoding the polypeptide to be operably linked to a promoter which, in some embodiments, is capable of driving the transcription of the SAMP coding sequence in plants. The promoter can be, e.g., derived from plant or viral sources. The promoter can be, e.g., constitutively active, inducible, or tissue specific. In construction of recombinant expression cassettes, vectors, transgenics, of the disclosure, a different promoter can be chosen and employed to differentially direct gene expression, e.g., in some or all tissues of a plant or animal.

Constitutive Promoters

A fragment can be employed to direct expression of a polynucleotide encoding the polypeptide in all transformed cells or tissues, e.g., as those of a transgenic plant. The term “constitutive regulatory element” means a regulatory element that confers a level of expression upon an operatively linked nucleic molecule that is relatively independent of the cell or tissue type in which the constitutive regulatory element is expressed. A constitutive regulatory element that is expressed in a plant generally is widely expressed in a large number of cell and tissue types. Promoters that drive expression continuously under physiological conditions are referred to as “constitutive” promoters and are active under most environmental conditions and states of development or cell differentiation.

A variety of constitutive regulatory elements useful for ectopic expression in a transgenic plant are well known in the art. The cauliflower mosaic virus 35S (CaMV 35S) promoter, for example, is a well-characterized constitutive regulatory element that produces a high level of expression in all plant tissues (Odell et al., Nature 313:810-812 (1985)). The CaMV 35S promoter can be particularly useful due to its activity in numerous diverse plant species (Benfey and Chua, Science 250:959-966 (1990); Futterer et al., Physiol. Plant 79:154 (1990); Odell et al., supra, 1985). A tandem 35S promoter, in which the intrinsic promoter element has been duplicated, confers higher expression levels in comparison to the unmodified 35S promoter (Kay et al., Science 236:1299 (1987)). Other useful constitutive regulatory elements include, for example, the cauliflower mosaic virus 19S promoter; the Figwort mosaic virus promoter; and the nopaline synthase (nos) gene promoter (Singer et al., Plant Mot. Biol. 14:433 (1990); An, Plant Physiol. 81:86 (1986)).

Additional constitutive regulatory elements including those for efficient expression in monocots also are known in the art, for example, the pEmu promoter and promoters based on the rice Actin-1 5′ region (Last et al, Theor. Appl. Genet. 81:581 (1991); Mcelroy et al, Mol Gen. Genet. 231:150 (1991); Mcelroy et al., Plant Cell 2:163 (1990)). Chimeric regulatory elements, which combine elements from different genes, also can be useful for ectopically expressing a nucleic acid molecule encoding a SAMP (e.g., an HS peptide) described herein (Comai et al., Plant Mol. Biol. 15:373 (1990)).

Other examples of constitutive promoters include the 1′- or 2′-promoter derived from T-DNA of Agrobacterium tumafaciens (see, e.g., Mengiste (1997) supra; O'Grady (1995) Plant Mol. Biot. 29:99-108); actin promoters, such as the Arabidopsis actin gene promoter (see, e.g., Huang (1997) Plant Mot. Biol. 1997 33:125-139); alcohol dehydrogenase (Adh) gene promoters (see, e.g., Millar (1996) Plant Mol. Biol. 31:897-904); ACT11 from Arabidopsis (Huang et at. Plant Mol Biol 33:125-139 (1996)), Cat3 from Arabidopsis (GenBank No. U43147, Zhong et al., Mol. Gen. Genet. 251:196-203 (1996)), the gene encoding stearoyl-acyl carrier protein desaturase from Brassica napus (Genbank No. X74782, Solocombe et al. Plant Physiol. 104:1167-1176 (1994)), GPc1 from maize (GenBank No. X15596, Martinez et al. J. Mol. Biol 208:551-565 (1989)), Gpc2 from maize (GenBank No. U45855, Manjunath et al., Plant Mot. Bio. 33:97-112 (1997)), other transcription initiation regions from various plant genes known to those of skill. See also Holtorf Plant Mo. Biol. 29:637-646 (1995).

Inducible Promoters

Alternatively, a plant promoter can direct expression of the polynucleotide encoding the SAMP (e.g., an HS peptide) under the influence of changing environmental conditions or developmental conditions. Examples of environmental conditions that can affect transcription by inducible promoters include anaerobic conditions, elevated temperature, drought, or the presence of light. Such promoters are referred to herein as “inducible” promoters. In some embodiments, an inducible promoter is one that is induced by one or more environmental stressors, including but not limited to, drought, freezing cold, and high salt. For example, the disclosure can incorporate a drought-specific promoter such as a drought-inducible promoter of maize (e.g., the maize rab17 drought-inducible promoter (Vilardell et al. (1991) Plant Mol. Biol. 17:985-993; Vilardell et al. (1994) Plant Mol. Biol. 24:561-569)); or alternatively a cold, drought, and high salt inducible promoter from potato (Kirch (1997) Plant Mol. Biol. 33:897-909) or from Arabidopsis (e.g., the rd29A promoter (Kasuga et al. (1999) Nature Biotechnology 17:287-291). Other environmental stress-inducible promoters include promoters from the following genes: Rab21, Wsi18, Lea3, Uge1, Dip1, and RIG1B in rice (Yi et al. (2010) Planta 232:743-754).

In some embodiments, a plant promoter is a stress-inducible promoter (e.g., a drought-, cold-, or salt-inducible promoter) that comprises a dehydration-responsive element (DRE) and/or an ABA-responsive element (ABRE), including but not limited to the rd29A promoter.

Alternatively, plant promoters which are inducible upon exposure to plant hormones, such as auxins, are used to express the polynucleotide encoding the polypeptide. For example, the disclosure can use the auxin-response elements E1 promoter fragment (AuxREs) in the soybean (Glycine max L.) (Liu (1997) Plant Physiol. 115:397407); the auxin-responsive Arabidopsis GST6 promoter (also responsive to salicylic acid and hydrogen peroxide) (Chen (1996) Plant J. 10: 955-966); the auxin-inducible parC promoter from tobacco (Sakai (1996) 37:906-913); a plant biotin response element (Streit (1997) Mol. Plant Microbe Interact. 10:933-937); and, the promoter responsive to the stress hormone abscisic acid (Sheen (1996) Science 274:1900-1902).

Plant promoters inducible upon exposure to chemicals reagents that can be applied to the plant, such as herbicides or antibiotics, are also useful for expressing the polynucleotide encoding the polypeptide. For example, the maize In2-2 promoter, activated by benzenesulfonamide herbicide safeners, can be used (De Veylder (1997) Plant Cell Physiol. 38:568-577); application of different herbicide safeners induces distinct gene expression patterns, including expression in the root, hydathodes, and the shoot apical meristem. A SAMP (e.g., an HS peptide) coding sequence can also be under the control of, e.g., a tetracycline-inducible promoter, e.g., as described with transgenic tobacco plants containing the Avena sativa L. (oat) arginine decarboxylase gene (Masgrau (1997) Plant J. 11:465-473); or, a salicylic acid-responsive element (Stange (1997) Plant J. 11:1315-1324; Uknes et al., Plant Cell 5:159-169 (1993); Bi et al., Plant J. 8:235-245 (1995)).

Examples of useful inducible regulatory elements include copper-inducible regulatory elements (Mett et al., Proc. Natl. Acad Sci. USA 90:4567-4571 (1993); Furst et al., Cell 55:705-717 (1988)); tetracycline and chlor-tetracycline-inducible regulatory elements (Gatz et al., Plant J. 2:397-404 (1992); Röder et al., Mol. Gen. Genet. 243:32-38 (1994); Gatz, Meth. Cell Biol. 50:411-424 (1995)); ecdysone inducible regulatory elements (Christopherson et al., Proc. Natl. Acad Sci. USA 89:6314-6318 (1992); Kreutzweiser et al., Ecotoxicol. Environ. Safety 28:14-24 (1994)); heat shock inducible regulatory elements (Takahashi et al., Plant Physiol. 99:383-390 (1992); Yabe et al., Plant Cell Physiol. 35:1207-1219 (1994); Ueda et al., Mol. Gen. Genet. 250:533-539 (1996)); and lac operon elements, which are used in combination with a constitutively expressed lac repressor to confer, for example. IPTG-inducible expression (Wilde et al., EMBO J. 11:1251-1259 (1992)). An inducible regulatory element useful in the transgenic plants of the disclosure also can be, for example, a nitrate-inducible promoter derived from the spinach nitrite reductase gene (Back et al., Plant Mol. Biol. 17:9 (1991)) or a light-inducible promoter, such as that associated with the small subunit of RuBP carboxylase or the LHCP gene families (Feinbaum et al., Mol. Gen. Genet. 226:449 (1991); Lam and Chua, Science 248:471 (1990)).

Tissue-Specific Promoters

Alternatively, the plant promoter can direct expression of the polynucleotide encoding the polypeptide in a specific tissue (tissue-specific promoters). Tissue specific promoters are transcriptional control elements that are only active in particular cells or tissues at specific times during plant development, such as in vegetative tissues or reproductive tissues.

Examples of tissue-specific promoters under developmental control include promoters that initiate transcription only (or primarily only) in certain tissues, such as vegetative tissues, e.g., roots or leaves, or reproductive tissues, such as fruit, ovules, seeds, pollen, pistols, flowers, or any embryonic tissue, or epidermis or mesophyll. Reproductive tissue-specific promoters can be, e.g., ovule-specific, embryo-specific, endosperm-specific. integument-specific, seed and seed coat-specific, pollen-specific, petal-specific, sepal-specific, or some combination thereof. In some embodiments, the promoter is cell-type specific, e.g., guard cell-specific.

Epidermal-specific promoters include, for example, the Arabidopsis LTP1 promoter (Thoma et al. (1994) Plant Physiol. 105(1):35-45), the CER1 promoter (Aarts et al. (1995) Plant Cell 7:2115-27), and the CER6 promoter (Hooker et al. (2002) Plant Physiol 129:1568-80), and the orthologous tomato LeCER6 (Vogg et al. (2004) J. Exp Bot. 55:1401-10).

Guard cell-specific promoters include, for example, the DGP1 promoter (Li et al. (2005) Science China C Life Sci. 48:181-186).

Other tissue-specific promoters include seed promoters. Suitable seed-specific promoters are derived from the following genes: MACI from maize (Sheridan (1996) Genetics 142:1009-1020); Cat3 from maize (GenBank No. L05934, Abler (1993) Plant Mol. Biol. 22:10131-1038); vivparous-1 from Arabidopsis (Genbank No. U93215); atmycl from Arabidopsis (Urao (1996) Plant Mol. Biol. 32:571-57; Conceicao (1994) Plant 5:493-505); napA from Brassica napus (GenBank No. J02798, Josefsson (1987) JBL 26:12196-1301); and the napin gene family from Brassica napus (Sjodahl (1995) Planta 197:264-271).

A variety of promoters specifically active in vegetative tissues, such as leaves, stems, roots and tubers, can also be used to express polynucleotide encoding polypeptides described herein. For example, promoters controlling patatin, the major storage protein of the potato tuber, can be used, see, e.g., Kim (1994) Plant Mol. Biol. 26:603-615; Martin (1997) Plant J. 11:53-62. The ORF13 promoter from Agrobacterium rhizogenes that exhibits high activity in roots can also be used (Hansen (1997) Mol. Gen. Genet. 254:337-343. Other useful vegetative tissue-specific promoters include: the tarin promoter of the gene encoding a globulin from a major taro (Colocasia esculenta L. Schott) corm protein family, tarin (Bezerra (1995) Plant Mol. Biol. 28:137-144); the curculin promoter active during taro corm development (de Castro (1992) Plant Cell 4:1549-1559) and the promoter for the tobacco root-specific gene TobRB7, whose expression is localized to root meristem and immature central cylinder regions (Yamamoto (1991) Plant Cell 3:371-382).

Leaf-specific promoters, such as the ribulose biphosphate carboxylase (RBCS) promoters, can also be used. For example, the tomato RBCS1, RBCS2 and RBCS3A genes are expressed in leaves and light-grown seedlings, only RBCS1 and RBCS2 are expressed in developing tomato fruits (Meier (1997) FEBS Lett. 415:91-95). Ribulose bisphosphate carboxylase promoters expressed almost exclusively in mesophyll cells in leaf blades and leaf sheaths at high levels, described by Matsuoka (1994) Plant J. 6:311-319, can be used. Another leaf-specific promoter is the light harvesting chlorophyll a/b binding protein gene promoter, see, e.g., Shiina (1997) Plant Physiol. 115:477-483; Casal (1998) Plant Physiol. 116:1533-1538. The Arabidopsis thaliana myb-related gene promoter (Atmyb5) described by Li (1996) FEBS Lett. 379:117-121, is leaf-specific. The Atmyb5 promoter is expressed in developing leaf trichomes, stipules, and epidermal cells on the margins of young rosette and cauline leaves, and in immature seeds. Atmyb5 mRNA appears between fertilization and the 16-cell stage of embryo development and persists beyond the heart stage. A leaf promoter identified in maize by Busk (1997)Plant J. 11:1285-1295, can also be used.

Another class of useful vegetative tissue-specific promoters are meristematic (root tip and shoot apex) promoters. For example, the “SHOOTMERISTEMLESS” and “SCARECROW” promoters, which are active in the developing shoot or root apical meristems, described by Di Laurenzio (1996) Cell 86:423-433; and, Long (1996) Nature 379:66-69; can be used. Another useful promoter is that which controls the expression of 3-hydroxy-3-methylglutaryl coenzyme A reductase HMG2 gene, whose expression is restricted to meristematic and floral (secretory zone of the stigma, mature pollen grains, gynoecium vascular tissue, and fertilized ovules) tissues (see, e.g., Enjuto (1995) Plant Cell. 7:517-527). Also useful are knl-related genes from maize and other species which show meristem-specific expression, see, e.g., Granger (1996) Plant Mol. Biol. 31:373-378; Kerstetter (1994) Plant Cell 6:1877-1887; Hake (1995) Philos. Trans. R. Soc. Lond. B. Biol. Sci. 350:45-51. For example, the Arabidopsis thaliana KNAT1 promoter (see, e.g., Lincoln (1994) Plant Cell 6:1859-1876).

One of skill will recognize that a tissue-specific promoter can drive expression of operably linked sequences in tissues other than the target tissue. Thus, as used herein a tissue-specific promoter is one that drives expression preferentially in the target tissue, but can also lead to some expression in other tissues as well.

In another embodiment, the polynucleotide encoding the polypeptide is expressed through a transposable element. This allows for constitutive, yet periodic and infrequent expression of the constitutively active polypeptide. The disclosure also provides for use of tissue-specific promoters derived from viruses including, e.g., the tobamovirus subgenomic promoter (Kumagai (1995) Proc. Natl. Acad. Sci. ISA 92:1679-1683; the rice tungro bacilliform virus (RTBV), which replicates only in phloem cells in infected rice plants, with its promoter which drives strong phloem-specific reporter gene expression; the cassava vein mosaic virus (CVMV) promoter, with highest activity in vascular elements, in leaf mesophyll cells, and in root tips (Verdaguer (1996) Plant Mol. Biol. 31:1129-1139).

In another embodiment, the present disclosure provides for expression vectors comprising an expression cassette of the disclosure (e.g., as described herein).

### VII. Plants

In some embodiments, the plant is a citrus plant. In some embodiments, the citrus plant is an orange tree, a lemon tree, a lime tree, or a grapefruit tree. In one embodiment, the citrus plant is a navel orange, Valencia orange, sweet orange, mandarin orange, or sour orange. In one embodiment, the citrus plant is a lemon tree. In one embodiment, the citrus plant is a lime tree. In some embodiments, the plant is a relative of a citrus plant (such as orange jasmine, limeberry, and trifoliate orange), or a plant that can be used as citrus rootstocks. In some embodiments, the plant is a potato plant.

In some embodiments, the present disclosure provides for plants (or a plant cell, seed, flower, leaf, fruit, or other plant part from such plants or processed food or food ingredient from such plants) comprising an expression cassette comprising a promoter operably linked to a polynucleotide encoding a SAMP (e.g., an HS peptide) of the disclosure (e.g., as described herein). In some embodiments, the plant has decreased UBC expression or activity and/or increased expression or activity of Pi transporters.

### VIII. Assessment of Antimicrobial Activity of SAMP Polypeptides; Production of SAMP Polypeptides Using Bacterial Expression Systems

In a further aspect, the disclosure provides methods of evaluating antimicrobial activity of SAMP polypeptides using an assay employing a culturable Liberibacter species, Liberibacter crescens, to assess anti-microbial activity, e.g., of candidate SAMP polypeptides.

In some embodiments, the SAMP polypeptide, e.g., a candidate SAMP polypeptide, is expressed using a bacterial expression system, e.g., E. coli, or a Bacillus sp. system, such as B. thuringiensis. B. megaterium, or B. subtilits, in order to rapidly and efficiently assess SAMP activity, e.g., of variants of SAMP polypeptides as described herein or of candidate antimicrobial polypeptides. Thus, for example, Gram-positive Bacillus spp. and Gram negative E. coli can be used to produce different types of potential antimicrobial polypeptides, the activity of which may then be evaluated using an L. crescens viability assay.

L. crescens is the sole culturable strain to date of the Liberibacter genus. However, there are difficulties in culturing L. crescens, such as long culture time and the low cell concentration, that make assay of antimicrobial of SAMPs using endpoints such as half maximal effective concentration (EC₅₀) or minimum inhibitory concentration (MIC) time-consuming and more difficult. However, by using an L. crescens viability assay, cultures needs not be re-established and dead cells can be directly viewed and quantified. By comparing the dead/live cell ratio, the antibacterial activity of molecules can be easily compared and used for large scale evaluation and screening.

Culture of L. crescens has been described (see, e.g., Naranjo et al., Scientific Reports 9:5150, 2019, https site doi.org/10.1038/s41598-019-41495-5; Sena-Velez et al., Applied and Environment. Microbiol. 85 Issue 21, e01656-19, 2019; and Jain et al., Phyopathology 109:1811-1819, 2019). In general, cells are culture using BM7 media. An illustrative culture protocol is provided in Example 6. In brief, cells are culture on BM7 or mBM7 solid medium and subcultured in BM7 or mBM7 broth medium for 1 week. The bacteria are collected and resuspended in a solutions, such as NACl, e.g., 150 mM NaCl for 10⁷ cell/ml, and treated for a desired period of time with a SAMP polypeptide, e.g., 1, 2, 3, 4, or 5 hours and a desired concentration, e.g., ranging from about 0.1 μM to about 50 μM. The proportion of dead vs. living cells can then be quantified using a bacterial cell viability assay. For example, a Viability/Cytotoxicity Assay kit for Bacteria Live and Dead Cells (available from Biotium) can be employed. This assay employs two fluorescent nucleic acid dyes: DMAO is a green-fluorescent nucleic acid dye that stains both live and dead bacteria. Ethidium Homodimer III (EthD-III) is a red-fluorescent nucleic acid dye that selectively stains dead bacteria with damaged cell membranes. When a bacterial preparation is stained with a mixture of DMAO and EthD-III, bacteria with intact cell membranes exhibit green fluorescence, whereas bacteria with damaged cell membranes exibit red fluorescence. Fluorescence can be determined using known assays, such as fluorescence microscopy.

Accordingly, in further aspects, provided herein is a method of evaluating SAMP activity, the method comprising incubating L. crescens cells with a polypeptide comprising one or more SAMP motifs, e.g., helix-2 as described herein; and analyzing the viability of the cells using a fluorescent viability assay that measure membrane integrity.

In a further aspect, provided herein is a method of producing a SAMP polypeptide the method comprising introducing an expression construct comprising a nucleic acid that encodes a SAMP polypeptide in an E. coli cell, or a Bacillus sp. bacterial cell; culturing the cell under conditions in which the SAMP polypeptide is expressed; and, optionally, isolating the SAMP polypeptide from the bacterial cell. In some embodiments, the bacterial cell is an E. coli cell. In some embodiments, the bacterial cell is a B. thuringiensis. B. megaterium, or B. subtilits cell. Thus, for example, in some embodiments, provided herein is a method of producing a SAMP polypeptide comprising introducing an expression construct comprising a nucleic acid that encodes a SAMP polypeptide into a B. thuringiensis cell; and culturing the B. thuringiensis cell under conditions in which the SAMP polypeptide is expressed. In some embodiments, the method further comprises isolating the SAMP polypeptide from the B. thuringiensis cell. In some embodiments, the nucleic acid encoding the SAMP polypeptide is operably linked to a Cyt1A or Cry3A promoter.

### IX. Illustrative Embodiments

The following embodiments are provided as example embodiments only. Embodiments of various aspects of the disclosure are not limited to these embodiments.

Embodiment 1: A polypeptide comprising a stable antimicrobial peptide (SAMP) that has an α-helical structure, wherein the SAMP comprises a sequence:

(SEQ ID NO: 53)
(V/S/A)H(V/L)E(F/Y)(A/T/S)(N/A/T)(L/E/I/S/T)
(F/L/M)(L/S)(A/S/P/G/T)(N/A/Q/S)(L/V/I)(E/D)K
(V/I/T/F)(L/I/V)(V/L/I)(I/L/V/F)DYK.

Embodiment 2. The polypeptide of Embodiment 1, comprising a sequence:

(SEQ ID NO: 54)
(V/S/A)H(V/L)E(F/Y)(A/T/S)N(L/E/I/S/T)(F/L/M)(L/S)
(A/S/P/G/T)(N/A/Q/S)(L/V/I)(E/D)K(V/I/T)(L/I/V)
(V/L/I)(I/L/V)DYK.

Embodiment 3: The polypeptide of Embodiment 2, comprising a sequence

(SEQ ID NO: 55)
(V/S/A)HVE(F/Y)AN(L/I/S/T)(F/L)(L/S)(A/P/)
(N/A/Q/S)LEKV(L/I)(V/L/I)(I/L/V)DYK.

Embodiment 4: The polypeptide of Embodiment 3, comprising a sequence of

(SEQ ID NO: 56)
(V/S/A)HVE(F/Y)AN(L/I/S/T)(F/L)LA(N/Q)LEKV(L/I)
(V/L/I)(I/L/V)DYK.

Embodiment 5: The polypeptide of Embodiment 1, comprising a sequence of any one of SEQ ID NOS:34-47, 50, 51, and 52.
Embodiment 6: A polypeptide comprising a sequence of SHVEYANLFLANLEKVLVIDYK (SEQ ID NO:34) or a variant thereof having at least 80%, 85%, 90%, or 95% identity to SEQ ID NO:34.
Embodiment 7: The polypeptide of Embodiment 6, wherein the variant comprises 1, 2, 3, or 4 substitutions relative to the sequence of SEQ ID NO:34.
Embodiment 8: A polypeptide comprising a stable antimicrobial peptide (SAMP) comprising a sequence of
HPX₁H(V/L)EX₂X₃X₄X₅X₆X₇X₈X₉X₁₀X₁₁KX₁₂X₁₃X₁₄X₁₅DX₁₆X₁₇X₁₈X₁₉X₂₀X₂₁X₂₂X₂₃X₂₄X₂₅X₂₆ (SEQ ID NO:31), or a fragment thereof,
wherein X₁ is S, A, or V;
X₂ is Y or F;
X₃, is A, S, or T;
X₄ is N, A, or T;
X₅ is L, T, I, S, or E;
X₆ is F, M, or L;
X₇ is L or S;
X₈ is A, P, T, G, or S;
X₉ is N, A, Q, S, or H;
X₁₀ is L, I, or V;
X₁₁ is E or D;
X₁₂ is V, I, F, or T;
X₁₃ is L, V, or I;
X₁₄ is V, L, or I;
X₁₅ is I, L, V, or F;
X₁₆ is Y or F;
X₁₇ is K or P;
X₁₈ is P or T;
X₁₉ is T, V, E, or Q;
X₂₀ is T, L, K, S, or absent;
X₂₁ is V, E, G, L, or absent;
X₂₂ is R, K, G, S, N, or absent;
X₂₃ is V, A, P, L, N, or absent;
X₂₄ is P, S, or absent;
X₂₅ is A or absent; and
X₂₆ is A or absent,
wherein the SAMP or a fragment thereof comprising a single α-helix structure.
   Embodiment 9: The polypeptide of Embodiment 8, wherein the SAMP does not have the sequence of

(SEQ ID NO: 33)
X₁GX₂X₃VSX₄ENX₅X₆QGFX₇HX₈FEX₉TFX₁₀SX₁₁EGX₁₂AEYX₁₃X₁₄,

wherein X₁ is R, K, or W; X₂ is K or E; X₃ is N or D; X₄ is T or I; X₅ is L, F, or R; X₆ is H or Q; X₇ is P or T; X₈ is I, L, or V; X₉ is S or F; X₁₀ is E or D; X₁₁ is T or L; X₁₂ is V or I; X₁₃ is V or I; X₁₄ is S, A, or D.
   Embodiment 10: The polypeptide of any one of Embodiments 1 to 8, wherein the SAMP has less than 67 amino acids.
   Embodiment 11: The polypeptide of any one of Embodiments 8 to 10, wherein the SAMP comprises a sequence of any one of

(SEQ ID NO: 17)
HPSHVEYANLFLANLEKVLVIDYKPTTVRV;
(SEQ ID NO: 18)
HPAHVEYANLFLANLEKVLVIDYKPTTVRV;
(SEQ ID NO: 19)
HPAHVEYANLFLANLEKVLVIDYKPTTERV;
(SEQ ID NO: 20)
HPSHVEFSATFSAAIEKIVLLDFPTVLGKAPAA;
(SEQ ID NO: 21)
HPVHVEFANLMLPQLEKVLVIDYKPEKVGP;
(SEQ ID NO: 22)
HPAHVEYANTLLPQLEKVLVIDYKPEKVGP;
(SEQ ID NO: 23)
HPVHVEYANTLLPQLEKFLIVDYKPQ;
(SEQ ID NO: 24)
HPAHVEYANILLTQLEKVLVIDYKPEKLSP;
(SEQ ID NO: 25)
HPVHVEFANTMLPQLEKVLIIDYKPQ;
(SEQ ID NO: 26)
HPAHVEYANSFLANLEKVLVIDYKPTTVRV;
(SEQ ID NO: 27)
HPAHVEYTNSFLANLEKVLVIDYKPTTVRV;
(SEQ ID NO: 28)
HPAHVEFATIFLGSLDKVLVIDYKPTSVSL;
(SEQ ID NO: 29)
HPAHVEFANEFLPALEKTLIIDYKPTSGNNS;
(SEQ ID NO: 30)
HPAHVEFANLFLSHVEKVIVFDYKPTTVRV,
and
(SEQ ID NO: 49)
HPVHLEFANLFLANLEKVLVIDYKPTTVRV,

or a fragment thereof comprising an α-helix structure.
   Embodiment 12: The polypeptide of any one of Embodiments 8 to 10, wherein the SAMP comprises 30 amino acids, the fifth amino acid in the 30-amino acid sequence is V, and wherein X₁ is A, V, or S; X₂ is Y or F; X₃ is A or T; X₄ is N or T; X₅ is I, T, L, or S; X₆ is L, M, or F; X₇ is L; X₈ is T, P, G, S, or A; X₉ is Q, S, H, or N; X₁₀ is L or V; X₁₁ is E or D; X₁₂ is V; X₁₃ is L or I; X₁₄ is V; X₁₅ is I or F; X₁₆ is Y; X₁₇ is K, X₁₈ is P; X₁₉ is E or T; X₂₀ is K, S, or T; X₂₁ L, V, or E; X₂₂ is S, G, or R; X₂₃ is P, L, or V; X₂₄ is absent; X₂₅ is absent; and X₂₆ is absent.
   Embodiment 13: The polypeptide of Embodiment 12, wherein the SAMP comprises a sequence of any one of

(SEQ ID NO: 17)
HPSHVEYANLFLANLEKVLVIDYKPTTVRV;
(SEQ ID NO: 18)
HPAHVEYANLFLANLEKVLVIDYKPTTVRV;
(SEQ ID NO: 19)
HPAHVEYANLFLANLEKVLVIDYKPTTERV;
(SEQ ID NO: 21)
HPVHVEFANLMLPQLEKVLVIDYKPEKVGP;
(SEQ ID NO: 22)
HPAHVEYANTLLPQLEKVLVIDYKPEKVGP;
(SEQ ID NO: 24)
HPAHVEYANILLTQLEKVLVIDYKPEKLSP;
(SEQ ID NO: 26)
HPAHVEYANSFLANLEKVLVIDYKPTTVRV;
(SEQ ID NO: 27)
HPAHVEYTNSFLANLEKVLVIDYKPTTVRV;
(SEQ ID NO: 28)
HPAHVEFATIFLGSLDKVLVIDYKPTSVSL;
and
(SEQ ID NO: 30)
HPAHVEFANLFLSHVEKVIVFDYKPTTVRV.

Embodiment 14: The polypeptide of any one of Embodiments 8 to 13, wherein X₁ is A or S; X₂ is Y; X₃ is A or T; X₄ is N; X₅ is L or S; X₆ is F; X₇ is L; X₈ is A; X₉ is N; X₁₀ is L; X₁₁ is E; X₁₂ is V; X₁₃ is L; X₁₄ is V; X₁₅ is I; X₁₆ is Y; X₁₇ is K, X₁₈ is P; X₁₉ is E; X₂₀ is K; X₂₁ is V or L; X₂₂ is G or S; X₂₃ is P; X₂₄ is absent; X₂₅ is absent; and X₂₆ is absent.
Embodiment 15: The polypeptide of Embodiment 14, wherein the SAMP comprises a sequence of any one of

(SEQ ID NO: 17)
HPSHVEYANLFLANLEKVLVIDYKPTTVRV;
(SEQ ID NO: 18)
HPAHVEYANLFLANLEKVLVIDYKPTTVRV;
(SEQ ID NO: 26)
HPAHVEYANSFLANLEKVLVIDYKPTTVRV;
and
(SEQ ID NO: 27)
HPAHVEYTNSFLANLEKVLVIDYKPTTVRV.

Embodiment 16: The polypeptide of any one of Embodiments 8 to 13, wherein X₁ is A or V; X₂ is F or Y; X₃ is A; X₄ is N; X₅ is I, L, or T; X₆ is L or M; X₇ is L; X₈ is P or T; X₉ is Q; X₁₀ is L; X₁₁ is E; X₁₂ is V; X₁₃ is L; X₁₄ is V; X₁₅ is I; X₁₆ is Y; X₁₇ is K, X₁₈ is P; X₁₉ is T; X₂₀ is T; X₂₁ is V; X₂₂ is R; X₂₃ is V; X₂₄ is absent; X₂₅ is absent; and X₂₆ is absent.
Embodiment 17: The polypeptide of Embodiment 16, wherein the SAMP comprises a sequence of any one of

(SEQ ID NO: 21)
HPVHVEFANLMLPQLEKVLVIDYKPEKVGP;
(SEQ ID NO: 22)
HPAHVEYANTLLPQLEKVLVIDYKPEKVGP;
and
(SEQ ID NO: 24)
HPAHVEYANILLTQLEKVLVIDYKPEKLSP.

Embodiment 18: The polypeptide of any one of Embodiments 8 to 13, wherein X₁ is A; X₂ is F or Y; X₃ is A; X₄ is T or N; X₅ is I or L; X₆ is F; X₇ is L; X₈ is G, S or A; X₉ is S, H, or N; X₁₀ is L or V; X₁₁ is D or E; X₁₂ is V; X₁₃ is L or I; X₁₄ is V; X₁₅ is I or F; X₁₆ is Y; X₁₇ is K, X₁₈ is P; X₁₉ is T; X₂₀ is T or S; X₂₁ is V or E; X₂₂ is S or R; X₂₃ is L or V; X₂₄ is absent; X₂₅ is absent; and X₂₆ is absent.
Embodiment 19: The polypeptide of Embodiment 19, wherein the SAMP comprises a sequence of any one of

(SEQ ID NO: 19)
HPAHVEYANLFLANLEKVLVIDYKPTTERV;
(SEQ ID NO: 28)
HPAHVEFATIFLGSLDKVLVIDYKPTSVSL;
and
(SEQ ID NO: 30)
HPAHVEFANLFLSHVEKVIVFDYKPTTVRV.

Embodiment 20. The polypeptide of any one of Embodiments 8 to 11, wherein the SAMP comprises 26 amino acids and wherein X₁ is V; X₂ is Y or F; X₃ is A; X₄ is N; X₅ is T; X₆ is L or M; X₇ is L; X₈ is P; X₉ is Q; X₁₀ is L; X₁₁ is E; X₁₂ is F or V; X₁₃ is L; X₁₄ is I; X₁₅ is V or I; X₁₆ is Y; X₁₇ is K, X₁₈ is P; X₁₉ is Q; X₂₀ is absent; X₂₁ is absent; X₂₂ is absent; X₂₃ is absent; X₂₄ is absent; X₂₅ is absent; and X₂₆ is absent.
Embodiment 21: The polypeptide of Embodiment 20, wherein the SAMP comprises a sequence of any one of

(SEQ ID NO: 23)
HPVHVEYANTLLPQLEKFLIVDYKPQ;
and
(SEQ ID NO: 25)
HPVHVEFANTMLPQLEKVLIIDYKPQ.

Embodiment 22: The polypeptide of any one of Embodiments 8 to 11, wherein the SAMP comprises a sequence of any one of

(SEQ ID NO: 20)
HPSHVEFSATFSAAIEKIVLLDFPTVLGKAPAA;
and
(SEQ ID NO: 29)
HPAHVEFANEFLPALEKTLIIDYKPTSGNNS.

Embodiment 23: A polypeptide comprising a stable antimicrobial peptide (SAMP) comprising a sequence of
HPAHVEFATIFLX₁X₂X₃X₄KX₅X₆X₇X₈DYKPTX₉X₁₀X₁₁X₁₂X₁₃ (SEQ ID NO:32), or a fragment thereof,
wherein X₁ is G, P, or S;
X₂ is S, A, or H;
X₃ is L or V;
X₄ is D or E;
X₅ is V or T;
X₆ is L or I;
X₇ is V or I;
X₈ is I or F;
X₉ is S or T;
X₁₀ V or G;
X₁₁ is S, N, or R;
X₁₂ is L, N, or V; and
X₁₃ is S or absent, wherein the SAMP or fragment thereof comprises a single α-helix structure.
   Embodiment 24: The polypeptide of Embodiment 23, wherein the SAMP comprises a sequence of any one of

(SEQ ID NO: 28)
HPAHVEFATIFLGSLDKVLVIDYKPTSVSL;
(SEQ ID NO: 29)
HPAHVEFANEFLPALEKTLIIDYKPTSGNNS;
and
(SEQ ID NO: 30)
HPAHVEFANLFLSHVEKVIVEDYKPTTVRV.

Embodiment 25: The polypeptide of any one of Embodiments 1 to 24, wherein the SAMP comprises between 20 and 24 amino acids.
Embodiment 26: The polypeptide of any one of Embodiments 1 to 25, wherein the SAMP is a heat stable (HS) peptide.
Embodiment 27: The polypeptide of any one of Embodiments 1 to 26, wherein the SAMP is stable in plant extracts and/or in plant lysates.
Embodiment 28: An agricultural composition comprising a polypeptide of any one of Embodiments 1 to 27.
Embodiment 29: The agricultural composition of Embodiment 28, further comprising at least one of an herbicide, an herbicide safener, a surfactant, a fungicide, a pesticide, a nematicide, a plant activator, a synergist, a plant growth regulator, an insect repellant, an acaricide, a molluscicide, or a fertilizer.
Embodiment 30: A nucleic acid molecule encoding a polypeptide of any one of Embodiments 1 to 27.
Embodiment 31: A cell comprising the nucleic acid molecule of Embodiment 30.
Embodiment 32: The cell of Embodiment 31, wherein the cell is a plant cell.
Embodiment 33: A plant comprising a polypeptide of any one of Embodiments 1 to 27 or the nucleic acid molecule of Embodiment 30.
Embodiment 34: The plant of Embodiment 33 wherein the plant is a citrus plant.
Embodiment 35: The plant of Embodiment 33, wherein the plant is a solanaceous plant.
Embodiment 36: A transgenic plant comprising an in situ mutated stable antimicrobial peptide (SAMP) comprising at least one amino acid substitution corresponding to an amino acid at any one of positions X₁ to X₂₆ as set forth in SEQ ID NO:31 or a substitution as set forth in SEQ ID NO:53, wherein the mutated SAMP comprises a single α-helix structure and wherein the mutated SAMP provides disease resistance or disease tolerance to the transgenic plant.
Embodiment 37: The transgenic plant of Embodiment 36, wherein the disease is a Liberibacter disease.
Embodiment 38: The transgenic plant of Embodiment 36, wherein the disease is an Agrobacterium or a Pseudomonas syringae disease.
Embodiment 39: The transgenic plant of Embodiment 37, wherein the Liberibacter disease is Huanglongbing (HLB).
Embodiment 40: The transgenic plant of any one of Embodiments 36 to 39, wherein the mutated SAMP is a heat stable (HS) peptide.
Embodiment 41: An expression cassette comprising a promoter operably linked to a polynucleotide encoding a polypeptide of any one of Embodiments 1 to 27, wherein introduction of the expression cassette into a plant results in the plant having enhanced disease resistance or disease tolerance.
Embodiment 42: The expression cassette of Embodiment 41, wherein the disease is a Liberibacter disease.
Embodiment 43: The expression cassette of Embodiment 41, wherein the disease is an Agrobacterium or a Pseudomonas syringae disease.
Embodiment 44: The expression cassette of Embodiment 42, wherein the Liberibacter disease is HLB.
Embodiment 45: The expression cassette of any one of Embodiments 41 to 44, wherein the promoter is heterologous to the polynucleotide.
Embodiment 46: The expression cassette of any one of Embodiments 41 to 45, wherein the promoter is inducible.
Embodiment 47: The expression cassette of any one of Embodiments 41 to 46, wherein the promoter is a tissue-specific promoter.
Embodiment 48: The expression cassette of Embodiment 47, wherein the tissue-specific promoter is a phloem-specific promoter.
Embodiment 49: The expression cassette of Embodiment 48, wherein phloem-specific promoter is the sucrose transporter protein SUC2 promoter.
Embodiment 50: A transgenic plant comprising the expression cassette of any one of Embodiments 41 to 49, wherein the plant has enhanced disease resistance or disease tolerance compared to a control plant lacking the expression cassette.
Embodiment 51: The transgenic plant of Embodiment 50, wherein the disease is a Liberibacter disease.
Embodiment 52: The transgenic plant of Embodiment 50, wherein the disease is an Agrobacterium or a Pseudomonas syringae disease.
Embodiment 53: The transgenic plant of Embodiment 51, wherein the Liberibacter disease is HLB.
Embodiment 54: The transgenic plant of any one of Embodiments 50 to 53, wherein the plant is a citrus plant.
Embodiment 55: The transgenic plant of any one of Embodiments 50 to 53, wherein the plant is a solanaceous plant.
Embodiment 56: A method of preventing or treating a bacterial disease in a plant, comprising contacting the plant with an exogenous polypeptide or with an agricultural composition that comprises the exogenous polypeptide or a polynucleotide encoding the exogenous polypeptide,
wherein, the exogenous polypeptide has an α-helical structure and comprises amino acid sequence

(SEQ ID NO: 53)
(V/S/A)H(V/L)E(F/Y)(A/T/S)(N/A/T)(L/E/I/S/T)
(F/L/M)(L/S)(A/S/P/G/T)(N/A/Q/S)(L/V/I)(E/D)K
(V/I/T/F)(L/I/V)(V/L/I)(I/L/V/F)DYK.

Embodiment 57: The method of Embodiment 56, wherein the bacterial disease is a Liberibacter-caused disease.
Embodiment 58: The method of Embodiment 56, wherein the bacterial disease is an Agrobacterium-caused or a Pseudomonas syringae-caused disease.
Embodiment 59: The method of Embodiment 57, wherein the Liberibacter-caused disease is HLB.
Embodiment 60: A method of preventing or treating a bacterial infection in a plant caused by bacteria in the genus Liberibacter, Agrobacterium, or Pseudomonas, comprising contacting the plant with a polypeptide of any one of Embodiments 1 to 27 or an agricultural composition of Embodiment 28 or 29.
Embodiment 61: The method of Embodiment 60 wherein the bacterium in the genus Liberibacter is Candidatus Liberibacter.
Embodiment 62: The method of Embodiment 60, wherein the bacterium in the genus Liberibacter is Liberibacter crescens.
Embodiment 63: The method of any one of Embodiments 60 to 62, wherein the polypeptide or agricultural composition is injected into the trunk of the plant.
Embodiment 64: The method of any one of Embodiments 60 to 62, wherein the polypeptide or agricultural composition is injected into the stem of the plant.
Embodiment 65: The method of any one of Embodiments 60 to 62, wherein the polypeptide or agricultural composition is applied by foliar spraying onto the plant or is applied to the roots by irrigation.
Embodiment 66: The method of any one of Embodiments 60 to 65, wherein the bacterial infection causes potato zebra chip disease.
Embodiment 67: A method of preventing or treating a Liberibacter, a Agrobacterium, or a Pseudomonas disease in a plant, comprising introducing an expression cassette of any one of Embodiments 41 to 49 into the plant.
Embodiment 68: The method of Embodiment 67, wherein the Liberibacter disease is HLB.
Embodiment 69: A method of preventing or treating a bacterial infection in a plant caused by a bacterium in the genus Liberibacter, Agrobacterium, or Pseudomonas, comprising introducing an expression cassette of any one of Embodiments 41 to 49 into the plant.
Embodiment 70: The method of Embodiment 69, wherein the bacterium in the genus Liberibacter is Candidatus Liberibacter.
Embodiment 71: The method of Embodiment 69, wherein the bacterium in the genus Liberibacter is Liberibacter crescens.
Embodiment 72: A method of producing a plant having enhanced Liberibacter, Agrobacterium, or Pseudomonas disease resistance or Liberibacter, Agrobacterium, or Pseudomonas disease tolerance, comprising:
introducing a polypeptide of any one of Embodiments 1 to 27 or an expression cassette of any one of Embodiments 41 to 49 into a plurality of plants; and
selecting a plant that comprises the polypeptide or expresses the polynucleotide from the plurality of plants.
   Embodiment 73: A method of producing a plant having enhanced Liberibacter, Agrobacterium, or Pseudomonas disease resistance or Liberibacter, Agrobacterium, or Pseudomonas disease tolerance, comprising introducing a mutation into an endogenous polynucleotide in the plant, wherein the mutated polynucleotide encodes a polypeptide of any one of Embodiments 1 to 27.
   Embodiment 74: The method of Embodiment 73, wherein the introducing occurs in situ in the genome of a plant cell.
   Embodiment 75: The method of Embodiment 73 or 74, wherein the introducing comprises genome editing.
   Embodiment 76: The method of any one of Embodiments 73 to 75, wherein the plant is resistant or tolerant to a bacterial infection caused by bacteria in the genus Liberibacter.
   Embodiment 77: The method of Embodiment 76, wherein the bacterium in the genus Liberibacter is Candidatus Liberibacter.
   Embodiment 78: The method of Embodiment 76, wherein the bacterium in the genus Liberibacter is Liberibacter crescens.
   Embodiment 79: The method of any one of Embodiments 73 to 78, wherein the Liberibacter disease is HLB.
   Embodiment 80: The method of any one of Embodiments 73 to 79, wherein the plant is a citrus plant.
   Embodiment 81: The method of any one of Embodiments 73 to 79, wherein the plant is a solanaceous plant.

### EXAMPLES

### Example 1—Treatment of Liberibacter crescens

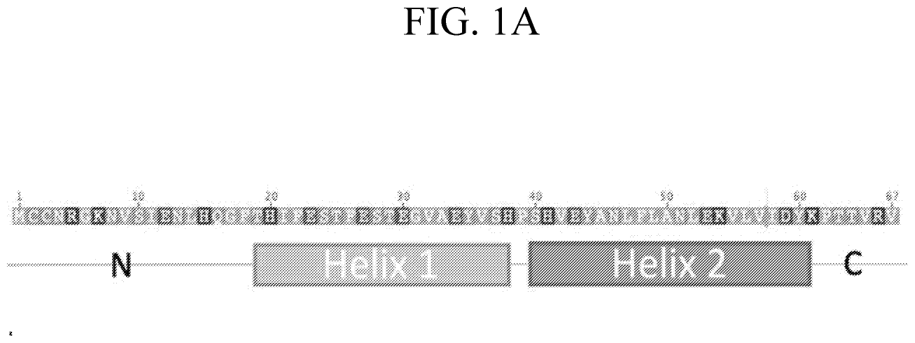

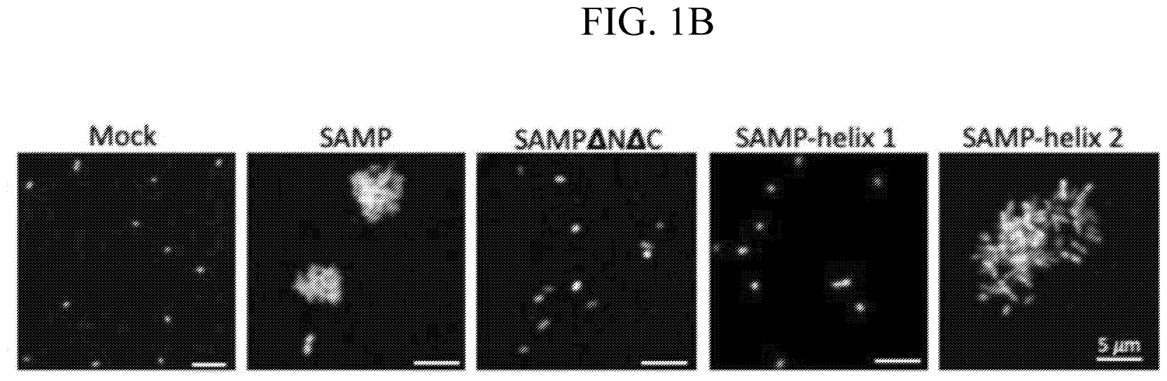

The culturable Liberibacter crescens (10⁶/mL) was treated with buffer (Mock), 30 μM of the full length of CghSAMPa (SEQ ID NO:1, referred to in FIG. 1B as SAMP); CghSAMPa with N-terminus and C-terminus deletions (referred to in FIG. 1B as SAMPANAC, HIFESTFESTEGVAEYVSHPSHVEYANLFLANLEKVLVIDYK (SEQ ID NO:57)); a polypeptide containing helix 1 of CghSAMPa (THIFESTFESTEGVAEYVSHP (SEQ ID NO:58), referred to as SAMP-helix 1 in FIG. 1B), or a polypeptide containing CghSAMPa helix 2 (SEQ ID NO:34, referred to as SAMP-helix 2 in FIG. 1B) for 2 hours. The cells were stained with viability staining and observed by Leica SP5 confocal microscopy (FIG. 1B). The results demonstrated that helix-2 plays an important role in anti-bacterial activity.

### Example 2—Effects of Increasing Concentration of SAMP

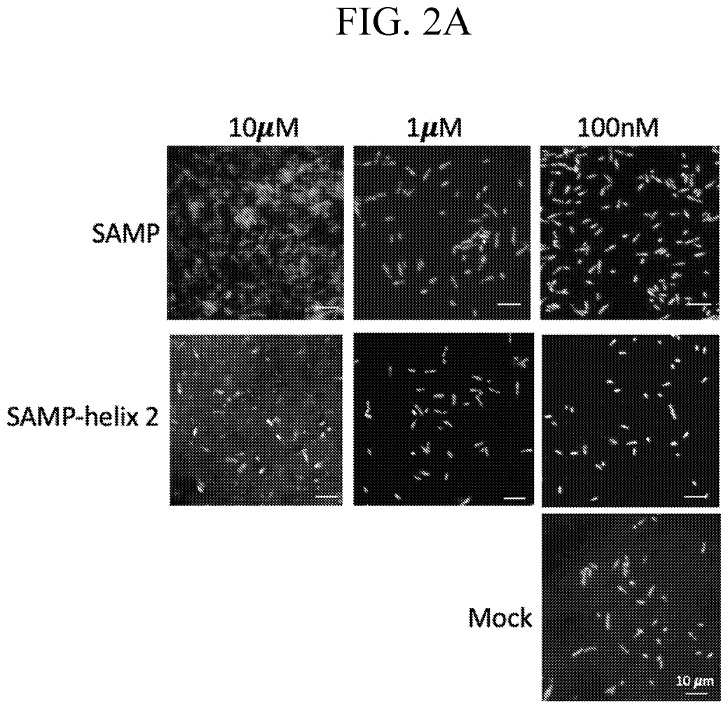

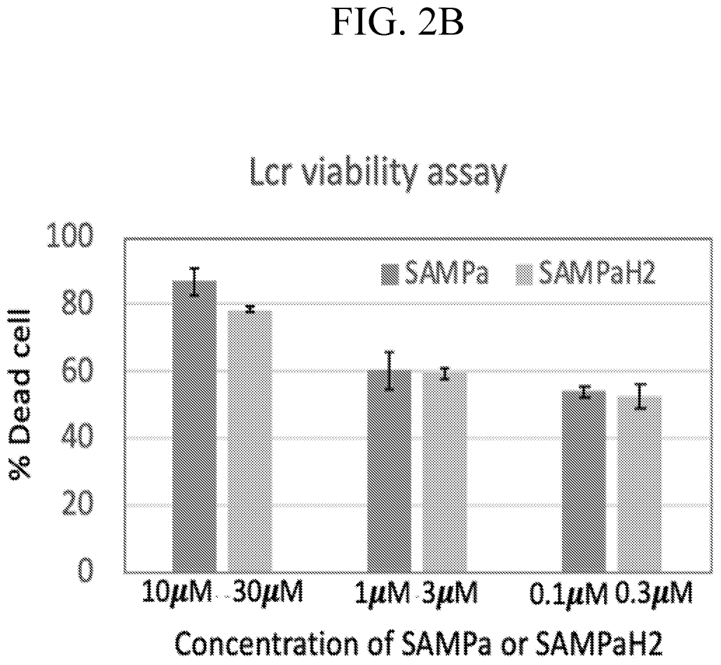

In FIG. 2A, the same concentration of full-length CghSAMPa (SEQ ID NO:1, referred to as SAMP) and CghSAMPa helix 2 (SEQ ID NO:34, referred to as SAMP-helix 2) were used to treat Liberibacter crescens (10⁶/ml) for 5 hours. The cells were stained with viability staining and observed by Leica SP5 confocal microscopy (FIG. 2A). FIG. 2B provides data from an evaluation in which triple dosages of CghSAMPa helix 2 (SEQ ID NO:34, SAMPaH2) compared to the doses of full-length CghSAMPa (SEQ ID NO:1. SAMPa) were used to treat Liberibacter crescens (10⁶/mL) for 5 hours. The cells were stained with viability staining and the fluorescence were measure by luminometer. The results demonstrated that CghSAMPa helix 2 retained much of the anti-bacterial activity of full length SAMP.

### Example 3—Expression Levels of Defense Marker Genes

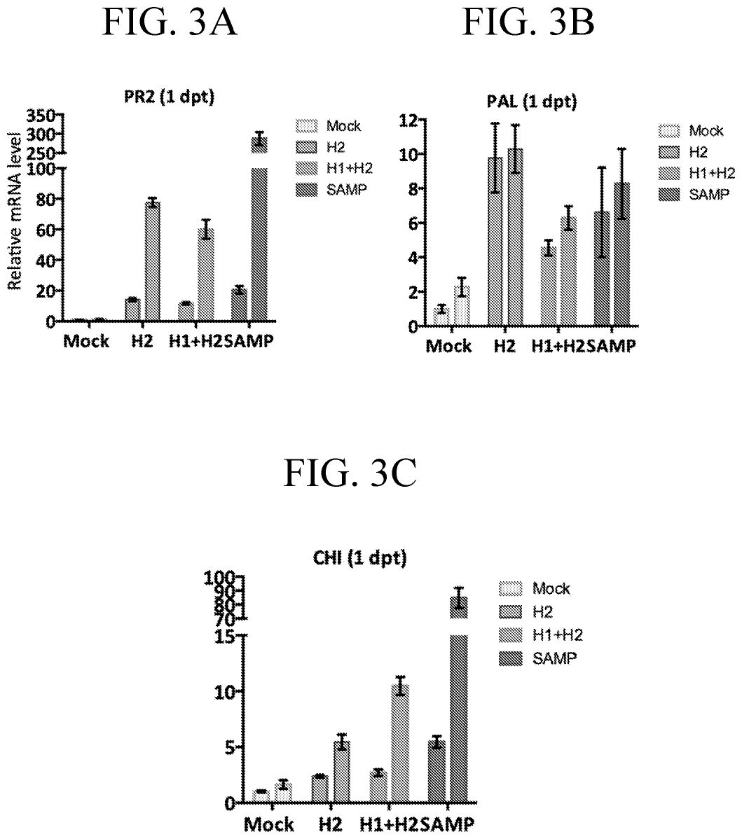

One and half years old Carrizo seedlings were sprayed (using a bottle sprayer) with buffer only (1× phosphate buffer saline (PBS) pH7.4 and Methylated Seed Oil Surfactant) (Mock), 10 μM SAMP-helix 2 (H2, SEQ ID NO:34), 5 μM SAMP-helix 1+5 μM SAMP-helix 2 (H1+H2, H1=SEQ ID NO:58), or SAMP full length (SAMP. SEQ ID NO:1). For this size of plant, one ml of the solution was sprayed on each plant. The expression levels of defense marker genes, including PR2, PAL, and CHI, were analyzed by qRT-PCR and normalized to the Ubiquitin gene. Expression was analyzed over a time course of up to 7 days post treatment. The illustrative data provided in FIGS. 3A-3C were obtained 1 day post-treatment. As shown in FIGS. 3A-3C, treatment of the plants with the exogenous polypeptide SAMP-helix 2 induced a defense response in Carrizo rootstock. Induction was maintained for the 7-day time period over which expression was evaluated.

Improvement in plant health following application of active SAMP may be determined as above, e.g., by measuring expression of defense response genes as an endpoint, but can also be indicated by phenotypic changes, such as emergence of young leaves that appear healthy after treatment.

### Example 4—Structural Analysis

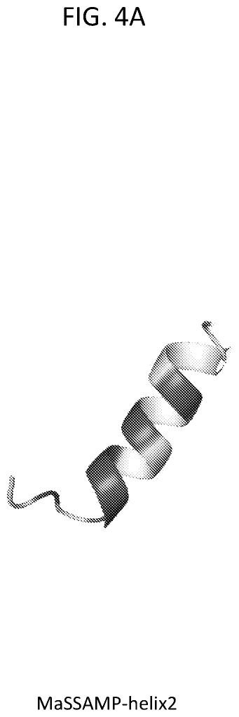

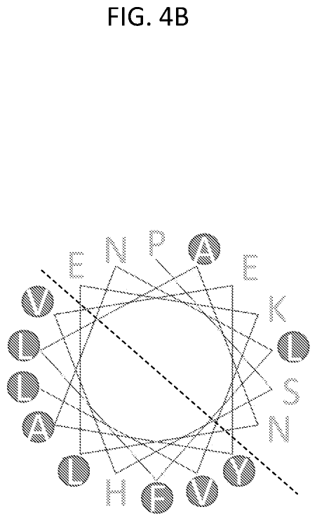

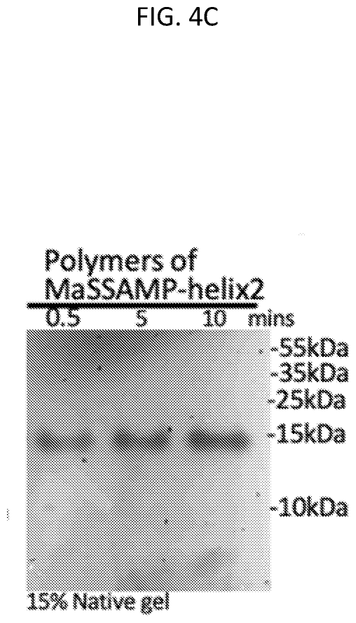

In FIGS. 4A-4C, MaSSAMP refers to CghSAMPa (SEQ ID NO:1) and MaSSAMP-helix2 refers to CghSAMPa-helix 2. To further examine the mechanism of action of MaSSAMP, we modeled its structure, which contains two short α-helical fragments connected by a proline hinge region with a loose N- and C-terminus. The amphipathic helix2 has hydrophobic residues facing one side and was predicted to form homo-dimers. Indeed, MaSSAMP polymers (likely hexamers based on the molecular weight) were detected in a native gel suggesting that this peptide forms a pore-like structure. Furthermore, the polymers (again, likely hexamers based on the molecular weight) were detected using only the helix2 domain (MaSSAMP-helix2), which further indicates that this peptide forms oligomers using via the helix 2 domain. FIG. 4A shows the structure prediction of MaSSAMP-helix2 by swiss-model. The hydrophobic residues are darker in color. FIG. 4B shows a helical wheel prediction diagram of MaSSAMP-helix 2. The hydrophobic residues are shown in squares. FIG. 4C depicts a native PAGE gel indicating that MaSSAMP and MaSSAMP-helix2 formed polymers, likely hexamers, in the native gel.

### Example 5—Evaluation of Tolerance of Bacterial Expression Systems

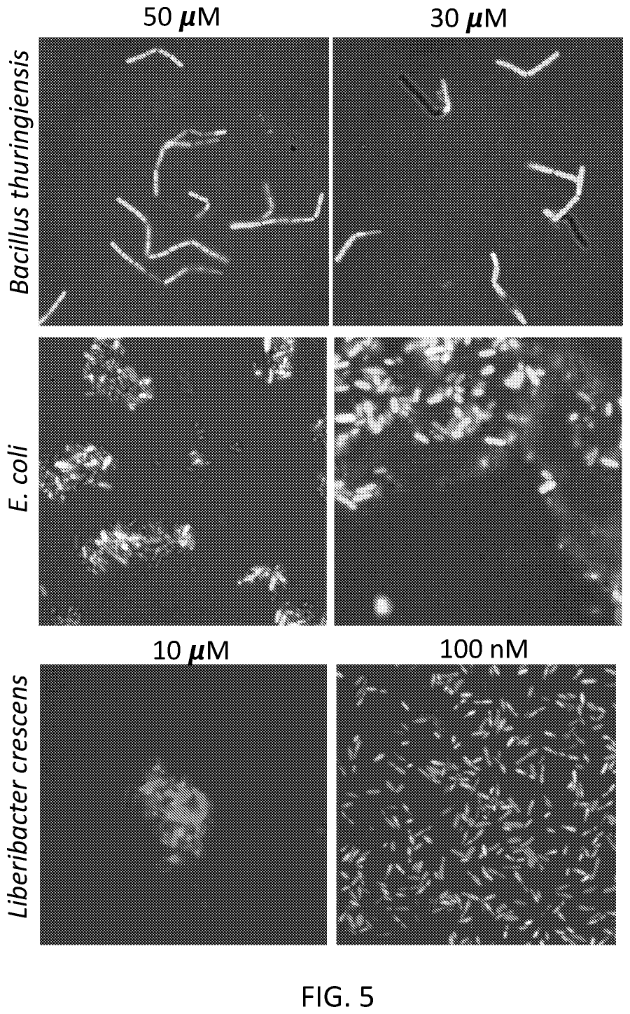

The tolerance of B. thuringiensis and E. coli to SAMP molecules was compared to that of Liberibacter crescens. Cells were subject to SAMP treatment at the concentrations shown in FIG. 5. The results demonstrated that both E. coli and B. thuringiensis are more tolerant of SAMP treatment compared to L. crescens. Indeed, B. thuringiensis was highly tolerant of SAMP. These results indicated that both E. coli and Bacillus sp. can be used to express SAMP polypeptides.

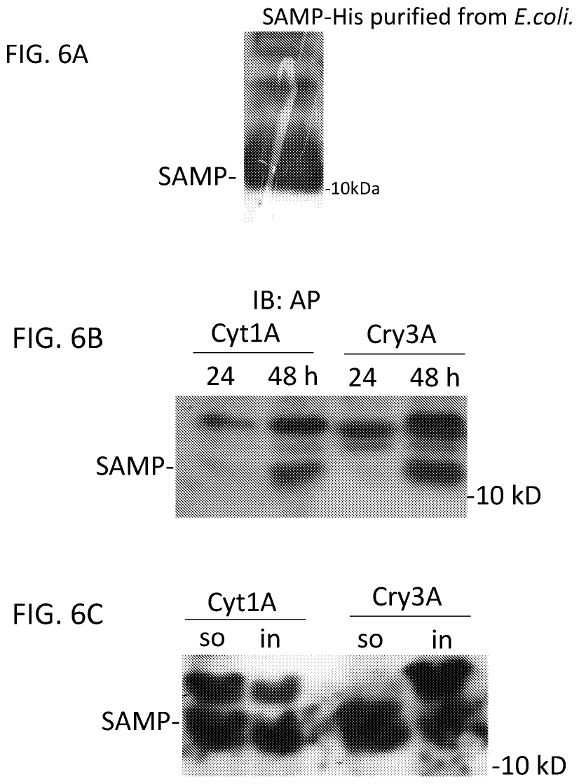

FIGS. 6A-6C provide data illustrating production of SAMP by B. thuringiensis and E. coli. FIG. 6A shows SAMP purified by NI-NTA column with 6×His tag from E. coli(C41). FIG. 6B shows SAMP production driven by promoter Cyt1A or Cry3A in 24- or 48-hr cultures of B. thuringiensis, as detected by western blot. FIG. 6C shows that SAMP is in the soluble fraction of cell lysates. The soluble and insoluble fraction of cell lysates from B. thuringiensis expressing SAMP driven by a Cyt1A or a Cry3A promoter were analyzed by western blot.

### Example 6—Illustrative Liberibacter crescens Cell Culture and Viability Assay

This examples provides a protocol that illustrates preparation of L. crescens cells for assay and a cell viability assay to assess SAMP activity towards L. crescens cells.

L. crescens Cells

Day 1—Streak L. crescens from glycerol stock on BM7 or mBM7 agar plates to produce a creamy lawn and Incubate at 28° C. in the dark for 7 days.

Day 8—Inoculate 20 ml liquid BM7 or mBM7 with a loopful of L. crescens culture and incubate 7 days in an orbital shaker at 150 rpm, 28° C. The OD₆₀₀ should be around 0.3-0.4. BM7 medim can be prepared as follows (see. Fagen et al 2014, publications available at https site doi.org/10.1371/journal.pone.0084469).

| Component | g/L |
|---|---|
| α-ketoglutarate | 2 |
| ACES Buffer | 10 |
| KOH | 3.75 |
| Water | 550 |

### Combine, pH to 6.9.

Autoclave @ 121° C. 30 minutes, cool to 60C and add:

| | |
|---|---|
| Fetal Bovine Serum | 150 |
| TMN-FH | 300 |

mBM7

| Component | g/L |
|---|---|
| α-ketoglutarate | 2 |
| ACES Buffer | 10 |
| KOH | 3.75 |
| 1-β methylcyclodextrin | 1 |
| Water | 700 |

### Combine, pH to 6.9.

Autoclave 121° C. 30 minutes cool to 60C and add:

| | |
|---|---|
| TMN-FH | 300 |

Liberibacter crescens Viability/Cytotoxicity Assay

This method can distinguish live and dead bacterial cell by cell membrane permeability upon treatment with antimicrobial peptides, antibiotics, antimicrobial molecules, etc.

A 100× dye solution is prepared according to the protocol from the manufacturer. AMO, EthD-111 and 150 mM NaCl are mixed at a ratio of 1:2:8. The stock solution dilution ratio may vary depending on the dyes used in the viability assays and/or the source of the reagents.

The L. crescens culture is centrifuged, e.g., at 5,000-10,000×g for 10-15 minutes, to collect the bacteria. The bacterial pellet in then resuspended in the 150 mM NaCl solution

The optical density at 600 nm (OD600) of the bacterial suspension is determined using a spectrophotometer and adjusted to 10⁸ bacteria-ml (OD600=1). The cell are then diluted 1:10 in 150 mM NaCl for a final density of 10⁷ bacteria/ml for the following treatment and staining.

Triton X-100 is added to the bacterial suspension as 1% for final concentration and incubate for 30 mins as a control for dead cells

The test molecule, e.g., SAMP polypeptide, is added to the bacterial suspension. The incubation time can vary, e.g., from 1 to 5 hours.

After treatment, 1/100 volume of 100× dye solution is added to the bacterial suspension and incubated for 15 mins.

Fluorescence of live (green fluorescence) and dead (red fluorescence) in the cell preparation can be observed or evaluated by using known technology, such as fluorescent microscopy, flow cytometry, or use of a luminometer; and the Dead/Live (or Live/Dead) cell ratio determined to evaluate the effect of antibacterial molecules.

One or more features from any embodiments described herein or in the figures can be combined with one or more features of any other embodiment described herein in the figures without departing from the scope of the disclosure.

All publications, patents and patent applications cited in this specification are herein incorporated by reference as if each individual publication or patent application were specifically and individually indicated to be incorporated by reference. Although the foregoing disclosure has been described in some detail by way of illustration and example for purposes of clarity of understanding, it will be readily apparent to those of ordinary skill in the art in light of the teachings of this disclosure that certain changes and modifications can be made thereto without departing from the spirit or scope of the appended claims.

What is claimed:

1. A method of preventing or treating a bacterial disease in a plant, comprising contacting the plant with an exogenous polypeptide or with an agricultural composition that comprises the exogenous polypeptide or a polynucleotide encoding the exogenous polypeptide,
wherein the exogenous polypeptide has an α-helical structure and comprises the amino acid sequence
(SEQ ID NO: 53)
(V/S/A)H(V/L)E(F/Y)(A/T/S)(N/A/T)(L/E/I/S/T)
(F/L/M)(L/S)(A/S/P/G/T)(N/A/Q/S)(L/V/I)(E/D)K
(V/I/T/F)(L/I/V)(V/L/I)(I/L/V/F)DYK.

2. The method of claim 1, wherein the exogenous polypeptide comprises the sequence SHVEYANLFLANLEKVLVIDYK (SEQ ID NO:34) or a variant thereof having at least 80%, 85%, 90%, or 95% identity to SEQ ID NO:34.

3. The method of claim 1, wherein the variant comprises 1, 2, 3, or 4 substitutions relative to the sequence of SEQ ID NO:34.

4. The method of claim 1, wherein the exogenous polypeptide comprises a sequence of any one of SEQ ID NOS:34-47, 50, 51, and 52.

5. The method of any one of claims 1 to 4, wherein the plant is contacted with an agricultural agent that comprises the exogenous polypeptide or a polynucleotide encoding the exogenous polypeptide, and wherein the agricultural agent is provided to the plant by spraying or dusting, delivery by root uptake, injection into plant's vascular system; or contacting the plant with cells comprising the exogenous polypeptide.

6. The method of any one of claims 1 to 4, wherein the bacterial disease is an Agrobacterium-caused or a Pseudomonas syringae-caused disease.

7. The method of any one of claims 1 to 4 bacterial disease is a Liberibacter-caused disease.

8. The method of claim 7, wherein the Liberibacter-caused disease is HLB.

9. A method of preventing or treating a bacterial infection in a plant caused by bacteria in the genus Liberibacter, Agrobacterium, or Pseudomonas, comprising contacting the plant with a polypeptide of any one of claims 14 to 40 or an agricultural composition of claim 41 or 42.

10. The method of claim 9, wherein the bacterium in the genus Liberibacter is Candidatus Liberibacter.

11. The method of claim 9, wherein the bacterium in the genus Liberibacter is Liberibacter crescens.

12. The method of any one of claims 1 to 11, wherein the exogenous polypeptide or agricultural composition is sprayed onto the plant or applied to the roots.

13. The method of any one of claims 9 to 12, wherein the bacterial infection causes potato zebra chip disease.

14. A polypeptide comprising a stable antimicrobial peptide (SAMP) that has an α-helical structure, wherein the SAMP comprises a sequence of
(SEQ ID NO: 53)
(V/S/A)H(V/L)E(F/Y)(A/T/S)(N/A/T)(L/E/I/S/T)
(F/L/M)(L/S)(A/S/P/G/T)(N/A/Q/S)(L/V/I)(E/D)K
(V/I/T/F)(L/I/V)(V/L/I)(I/L/V/F)DYK.

15. The polypeptide of claim 14, comprising a sequence of
(SEQ ID NO: 54)
(V/S/A)H(V/L)E(F/Y)(A/T/S)N(L/E/I/S/T)(F/L/M)(L/S)
(A/S/P/G/T)(N/A/Q/S)(L/V/I)(E/D)K(V/I/T)(L/I/V)
(V/L/I)(I/L/V)DYK.

16. The polypeptide of claim 15, comprising a sequence of
(SEQ ID NO: 55)
(V/S/A)HVE(F/Y)AN(L/I/S/T)(F/L)(L/S)(A/P/)
(N/A/Q/S)LEKV(L/I)(V/L/I)(I/L/V)DYK.

17. The polypeptide of claim 16, comprising a sequence of
(SEQ ID NO: 56)
(V/S/A)HVE(F/Y)AN(L/I/S/T)(F/L)LA(N/Q)LEKV(L/I)
(V/L/I)(I/L/V)DYK.

18. The polypeptide of claim 14, comprising a sequence of any one of SEQ ID NOS:34-47, 50, 51, and 52.

19. A polypeptide comprising a sequence of SHVEYANLFLANLEKVLVIDYK (SEQ ID NO:34) or a variant thereof having at least 80%, 85%, 90%, or 95% identity to SEQ ID NO:34.

20. The polypeptide of claim 19, wherein the variant comprises 1, 2, 3, or 4 substitutions relative to the sequence of SEQ ID NO:34.

21. A polypeptide comprising a stable antimicrobial peptide (SAMP) comprising a sequence of
HPX₁H(V/L)EX₂X₃X₄X₅X₆X₇X₈X₉X₁₀X₁₁X₁₂X₁₃X₁₄X₁₅X₁₆X₁₇X₁₈X₁₉X₂₀X₂₁X₂₂X₂₃X₂₄X₂₅X₂₆ (SEQ ID NO:31), or a fragment thereof,
wherein X₁ is S, A, or V;
X₂ is Y or F;
X₃, is A, S, or T;
X₄ is N, A, or T;
X₅ is L, T, I, S, or E;
X₆ is F, M, or L;
X₇ is L or S;
X₈ is A, P, T, G, or S;
X₉ is N, A, Q, S, or H;
X₁₀ is L, I, or V;
X₁₁ is E or D;
X₁₂ is V, I, F, or T;
X₁₃ is L, V, or I;
X₁₄ is V, L, or I;
X₁₅ is I, L, V, or F;
X₁₆ is Y or F;
X₁₇ is K or P;
X₁₈ is P or T;
X₁₉ is T, V, E, or Q;
X₂₀ is T, L, K, S, or absent;
X₂₁ is V, E, G, L, or absent;
X₂₂ is R, K, G, S, N, or absent;
X₂₃ is V, A, P, L, N, or absent;
X₂₄ is P, S, or absent;
X₂₅ is A or absent; and
X₂₆ is A or absent,
wherein the SAMP or a fragment thereof comprising a single α-helix structure.

22. The polypeptide of claim 21, wherein the SAMP does not have the sequence of
(SEQ ID NO: 33)
X₁GX₂X₃VS₄ENX₅X₆QGFX₇HX₈FEX₉TFX₁₀SX₁₁EGX₁₂AEYX₁₃X₁₄,
wherein X₁ is R, K, or W; X₂ is K or E; X₃ is N or D; X₄ is T or I; X₅ is L, F, or R; X₆ is H or Q; X₇ is P or T; X₈ is 1, L, or V; X₉ is S or F; X₁₀ is E or D; X₁₁ is T or L; X₁₂ is V or I; X₁₃ is V or I; X₁₄ is S, A, or D.

23. The polypeptide of claim 14 to 22, wherein the SAMP has less than 67 amino acids.

24. The polypeptide of claim any one of claims 21 to 23, wherein the SAMP comprises a sequence of any one of
(SEQ ID NO: 17)
HPSHVEYANLFLANLEKVLVIDYKPTTVRV;
(SEQ ID NO: 18)
HPAHVEYANLFLANLEKVLVIDYKPTTVRV;
(SEQ ID NO: 19)
HPAHVEYANLFLANLEKVLVIDYKPTTERV;
(SEQ ID NO: 20)
HPSHVEFSATFSAAIEKIVLLDFPTVLGKAPAA;
(SEQ ID NO: 21)
HPVHVEFANLMLPQLEKVLVIDYKPEKVGP;
(SEQ ID NO: 22)
HPAHVEYANTLLPQLEKVLVIDYKPEKVGP;
(SEQ ID NO: 23)
HPVHVEYANTLLPQLEKFLIVDYKPQ;
(SEQ ID NO: 24)
HPAHVEYANILLTQLEKVLVIDYKPEKLSP;
(SEQ ID NO: 25)
HPVHVEFANTMLPQLEKVLIIDYKPQ;
(SEQ ID NO: 26)
HPAHVEYANSFLANLEKVLVIDYKPTTVRV;
(SEQ ID NO: 27)
HPAHVEYTNSFLANLEKVLVIDYKPTTVRV;
(SEQ ID NO: 28)
HPAHVEFATIFLGSLDKVLVIDYKPTSVSL;
(SEQ ID NO: 29)
HPAHVEFANEFLPALEKTLIIDYKPTSGNNS;
(SEQ ID NO: 30)
HPAHVEFANLFLSHVEKVIVEDYKPTTVRV,
and
(SEQ ID NO: 49)
HPVHLEFANLFLANLEKVLVIDYKPTTVRV,
or a fragment thereof comprising an α-helix structure.

25. The polypeptide of any one of claims 21 to 23, wherein the SAMP comprises 30 amino acids, the fifth amino acid in the 30-amino acid sequence is V, and wherein X₁ is A, V, or S; X₂ is Y or F; X₃ is A or T; X₄ is N or T; X₅ is I, T, L, or S; X₆ is L, M, or F; X₇ is L; X₈ is T, P, G, S, or A; X₉ is Q, S, H, or N; X₁₀ is L or V; X₁₁ is E or D; X₁₂ is V; X₁₃ is L or I; X₁₄ is V; X₁₅ is I or F; X₁₆ is Y; X₁₇ is K, X₁₈ is P; X₁₉ is E or T; X₂₀ is K, S, or T; X₂₁ L, V, or E; X₂₂ is S, G, or R; X₂₃ is P, L, or V; X₂₄ is absent; X₂₅ is absent; and X₂₆ is absent.

26. The polypeptide of claim 25, wherein the SAMP comprises a sequence of any one of
(SEQ ID NO: 17)
HPSHVEYANLFLANLEKVLVIDYKPTTVRV;
(SEQ ID NO: 18)
HPAHVEYANLFLANLEKVLVIDYKPTTVRV;
(SEQ ID NO: 19)
HPAHVEYANLFLANLEKVLVIDYKPTTERV;
(SEQ ID NO: 21)
HPVHVEFANLMLPQLEKVLVIDYKPEKVGP;
(SEQ ID NO: 22)
HPAHVEYANTLLPQLEKVLVIDYKPEKVGP;
(SEQ ID NO: 24)
HPAHVEYANILLTQLEKVLVIDYKPEKLSP;
(SEQ ID NO: 26)
HPAHVEYANSFLANLEKVLVIDYKPTTVRV;
(SEQ ID NO: 27)
HPAHVEYTNSFLANLEKVLVIDYKPTTVRV;
(SEQ ID NO: 28)
HPAHVEFATIFLGSLDKVLVIDYKPTSVSL;
and
(SEQ ID NO: 30)
HPAHVEFANLFLSHVEKVIVEDYKPTTVRV.

27. The polypeptide of any one of claims 21 to 23, wherein X₁ is A or S; X₂ is Y; X₃ is A or T; X₄ is N; X₅ is L or S; X₆ is F; X₇ is L; X₈ is A; X₉ is N; X₁₀ is L; X₁₁ is E; X₁₂ is V; X₁₃ is L; X₁₄ is V; X₁₅ is 1; X₁₆ is Y; X₁₇ is K, X₁₈ is P; X₁₉ is E; X₂₀ is K; X₂₁ is V or L; X₂₂ is G or S; X₂₃ is P; X₂₄ is absent; X₂₅ is absent; and X₂₆ is absent.

28. The polypeptide of claim 27, wherein the SAMP comprises a sequence of any one of
(SEQ ID NO: 17)
HPSHVEYANLFLANLEKVLVIDYKPTTVRV;
(SEQ ID NO: 18)
HPAHVEYANLFLANLEKVLVIDYKPTTVRV;
(SEQ ID NO: 26)
HPAHVEYANSFLANLEKVLVIDYKPTTVRV;
and
(SEQ ID NO: 27)
HPAHVEYTNSFLANLEKVLVIDYKPTTVRV.

29. The polypeptide of any one of claims 21 to 23, wherein X₁ is A or V; X₂ is F or Y; X₃ is A; X₄ is N; X₅ is I, L, or T; X₆ is L or M; X₇ is L; X₈ is P or T; X₉ is Q; X₁₀ is L; X₁₁ is E; X₁₂ is V; X₁₃ is L; X₁₄ is V; X₁₅ is I; X₁₆ is Y; X₁₇ is K, X₁₈ is P; X₁₉ is T; X₂₀ is T; X₂₁ is V; X₂₂ is R; X₂₃ is V; X₂₄ is absent; X₂₅ is absent; and X₂₆ is absent.

30. The polypeptide of claim 29, wherein the SAMP comprises a sequence of any one of
(SEQ ID NO: 21)
HPVHVEFANLMLPQLEKVLVIDYKPEKVGP;
(SEQ ID NO: 22)
HPAHVEYANTLLPQLEKVLVIDYKPEKVGP;
and
(SEQ ID NO: 24)
HPAHVEYANILLTQLEKVLVIDYKPEKLSP.

31. The polypeptide of any one of claims 21 to 23, wherein X₁ is A; X₂ is F or Y; X₃ is A; X₄ is T or N; X₅ is I or L; X₆ is F; X₇ is L; X₈ is G, S or A; X₉ is S, H, or N; X₁₀ is L or V; X₁₁ is D or E; X₁₂ is V; X₁₃ is L or I; X₁₄ is V; X₁₅ is I or F; X₁₆ is Y; X₁₇ is K, X₁₈ is P; X₁₉ is T; X₂₀ is T or S; X₂₁ is V or E; X₂₂ is S or R; X₂₃ is L or V; X₂₄ is absent; X₂₅ is absent; and X₂₆ is absent.

32. The polypeptide of claim 31, wherein the SAMP comprises a sequence of any one of
(SEQ ID NO: 19)
HPAHVEYANLFLANLEKVLVIDYKPTTERV;
(SEQ ID NO: 28)
HPAHVEFATIFLGSLDKVLVIDYKPTSVSL;
and
(SEQ ID NO: 30)
HPAHVEFANLFLSHVEKVIVFDYKPTTVRV.

33. The polypeptide of any one of claims 21 to 23, wherein the SAMP comprises 26 amino acids and wherein X₁ is V; X₂ is Y or F; X₃ is A; X₄ is N; X₅ is T; X₆ is L or M; X₇ is L; X₈ is P; X₉ is Q; X₁₀ is L; X₁₁ is E; X₁₂ is F or V; X₁₃ is L; X₁₄ is I; X₁₅ is V or I; X₁₆ is Y; X₁₇ is K, X₁₈ is P; X₁₉ is Q; X₂₀ is absent; X₂₁ is absent; X₂₂ is absent; X₂₃ is absent; X₂₄ is absent; X₂₅ is absent; and X₂₆ is absent.

34. The polypeptide of claim 33, wherein the SAMP comprises a sequence of any one of
(SEQ ID NO: 23)
HPVHVEYANTLLPQLEKFLIVDYKPQ;
and
(SEQ ID NO: 25)
HPVHVEFANTMLPQLEKVLIIDYKPQ.

35. The polypeptide of any one of claims 21 to 23, wherein the SAMP comprises a sequence of any one of
(SEQ ID NO: 20)
HPSHVEFSATFSAAIEKIVLLDFPTVLGKAPAA;
and
(SEQ ID NO: 29)
HPAHVEFANEFLPALEKTLIIDYKPTSGNNS.

36. A polypeptide comprising a stable antimicrobial peptide (SAMP) comprising a sequence of
HPAHVEFATIFLX₁X₂X₃X₄KX₅X₆X₇X₈DYKPTX₉X₁₀X₁₁X₁₂X₁₃ (SEQ ID NO:32), or a fragment thereof,
wherein X₁ is G, P, or S;
X₂ is S, A, or H;
X₃ is L or V;
X₄ is D or E;
X₅ is V or T;
X₆ is L or I;
X₇ is V or I;
X₈ is I or F;
X₉ is S or T;
X₁₀ V or G;
X₁₁ is S, N, or R;
X₁₂ is L, N, or V; and
X₁₃ is S or absent, wherein the SAMP or fragment thereof comprises a single α-helix structure.

37. The polypeptide of claim 36, wherein the SAMP comprises a sequence of any one of
(SEQ ID NO: 28)
HPAHVEFATIFLGSLDKVLVIDYKPTSVSL;
(SEQ ID NO: 29)
HPAHVEFANEFLPALEKTLIIDYKPTSGNNS;
and
(SEQ ID NO: 30)
HPAHVEFANLFLSHVEKVIVFDYKPTTVRV.

38. The polypeptide of any one of claims 14 to 37, wherein the SAMP comprises between 20 and 24 amino acids.

39. The polypeptide of any one of claims 14 to 38, wherein the SAMP is a heat stable (HS) peptide.

40. The polypeptide of any one of claims 14 to 39, wherein the SAMP is stable in plant extracts and/or in plant lysates.

41. An agricultural composition comprising a polypeptide of any one of claims 14 to 40.

42. The agricultural composition of claim 41, further comprising at least one of an herbicide, an herbicide safener, a surfactant, a fungicide, a pesticide, a nematicide, a plant activator, a synergist, a plant growth regulator, an insect repellant, an acaricide, a molluscicide, or a fertilizer.

43. A nucleic acid molecule encoding a polypeptide of any one of claims 14 to 40.

44. A cell comprising the nucleic acid molecule of claim 43.

45. The cell of claim 44, wherein the cell is a plant cell.

46. A plant comprising a polypeptide of any one of claims 14 to 40 or the nucleic acid molecule of claim 43.

47. The plant of claim 46, wherein the plant is a citrus plant.

48. The plant of claim 46, wherein the plant is a solanaceous plant.

49. A transgenic plant comprising an in situ mutated stable antimicrobial peptide (SAMP) comprising at least one amino acid substitution corresponding to an amino acid at any one of positions X₁ to X₂₆ as set forth in SEQ ID NO:31, wherein the mutated SAMP comprises a single α-helix structure and wherein the mutated SAMP provides disease resistance or disease tolerance to the transgenic plant.

50. The transgenic plant of claim 49, wherein the disease is a Liberibacter disease.

51. The transgenic plant of claim 49, wherein the disease is an Agrobacterium or a Pseudomonas syringae disease.

52. The transgenic plant of claim 50, wherein the Liberibacter disease is Huanglongbing (HLB).

53. The transgenic plant of any one of claims 49 to 52, wherein the mutated SAMP is a heat stable (HS) peptide.

54. An expression cassette comprising a promoter operably linked to a polynucleotide encoding a polypeptide of any one of claims 14 to 40, wherein introduction of the expression cassette into a plant results in the plant having enhanced disease resistance or disease tolerance.

55. The expression cassette of claim 54, wherein the disease is a Liberibacter disease.

56. The expression cassette of claim 54, wherein the disease is an Agrobacterium or a Pseudomonas syringae disease.

57. The expression cassette of claim 55, wherein the Liberibacter disease is HLB.

58. The expression cassette of any one of claims 54 to 57, wherein the promoter is heterologous to the polynucleotide.

59. The expression cassette of any one of claims 54 to 58, wherein the promoter is inducible.

60. The expression cassette of any one of claims 54 to 59, wherein the promoter is a tissue-specific promoter.

61. The expression cassette of claim 60, wherein the tissue-specific promoter is a phloem-specific promoter.

62. The expression cassette of claim 61, wherein phloem-specific promoter is the sucrose transporter protein SUC2 promoter.

63. A transgenic plant comprising the expression cassette of any one of claims 54 to 62, wherein the plant has enhanced disease resistance or disease tolerance compared to a control plant lacking the expression cassette.

64. The transgenic plant of claim 63, wherein the disease is a Liberibacter disease.

65. The transgenic plant of claim 63, wherein the disease is an Agrobacterium or a Pseudomonas syringae disease.

66. The transgenic plant of claim 64, wherein the Liberibacter disease is HLB.

67. The transgenic plant of any one of claims 63 to 66, wherein the plant is a citrus plant.

68. The transgenic plant of any one of claims 63 to 66, wherein the plant is a solanaceous plant.

69. A method of preventing or treating a Liberibacter, a Agrobacterium, or a Pseudomonas disease in a plant, comprising introducing an expression cassette of any one of claims 54 to 62 into the plant.

70. The method of claim 69, wherein the Liberibacter disease is HLB.

71. A method of preventing or treating a bacterial infection in a plant caused by a bacterium in the genus Liberibacter, Agrobacterium, or Pseudomonas, comprising introducing an expression cassette of any one of claims 54 to 62 into the plant.

72. The method of claim 71, wherein the bacterium in the genus Liberibacter is Candidatus Liberibacter.

73. The method of claim 71, wherein the bacterium in the genus Liberibacter is Liberibacter crescens.

74. A method of producing a plant having enhanced Liberibacter, Agrobacterium, or Pseudomonas disease resistance or Liberibacter, Agrobacterium, or Pseudomonas disease tolerance, comprising:
introducing a polypeptide of any one of claims 14 to 40 or an expression cassette of any one of claims 54 to 62 into a plurality of plants; and
selecting a plant that comprises the polypeptide or expresses the polynucleotide from the plurality of plants.

75. A method of producing a plant having enhanced Liberibacter, Agrobacterium, or Pseudomonas disease resistance or Liberibacter, Agrobacterium, or Pseudomonas disease tolerance, comprising introducing a mutation into an endogenous polynucleotide in the plant, wherein the mutated polynucleotide encodes a polypeptide of any one of claims 14 to 40.

76. The method of claim 75, wherein the introducing occurs in situ in the genome of a plant cell.

77. The method of claim 75 or 76, wherein the introducing comprises genome editing.

78. The method of any one of claims 75 to 77, wherein the plant is resistant or tolerant to a bacterial infection caused by bacteria in the genus Liberibacter.

79. The method of claim 78, wherein the bacterium in the genus Liberibacter is Candidatus Liberibacter.

80. The method of claim 78, wherein the bacterium in the genus Liberibacter is Liberibacter crescens.

81. The method of any one of claims 75 to 80, wherein the Liberibacter disease is HLB.

82. The method of any one of claims 75 to 81, wherein the plant is a citrus plant.

83. The method of any one of claims 75 to 81, wherein the plant is a solanaceous plant.


SEQUENCE LISTING (60 sequences)

SEQ ID NO: 1 - protein, 67 aa, Unknown
  Description of Unknown SAMP sequence
MCCNRGKNVSIENLHQGFTHIFESTFESTEGVAEYVSHPSHVEYANLFLANLEKVLVIDYKPTTVRV

SEQ ID NO: 2 - protein, 67 aa, Unknown
  Description of Unknown SAMP sequence
MCCNRGKNVSIENLHQGFPHLFEFTFESTEGVAEYVSHPAHVEYANLFLANLEKVLVIDYKPTTVRV

SEQ ID NO: 3 - protein, 67 aa, Unknown
  Description of Unknown SAMP sequence
MCCNRGKNVSIENLHQGFTHIFESTFESTEGVAEYVAHPAHVEYANLFLANLEKVLVIDYKPTTERV

SEQ ID NO: 4 - protein, 67 aa, Unknown
  Description of Unknown SAMP sequence
MCCNRGKNVSIENLHQGFTHIFESTFESTEGVAEYVAHPAHVEYANLFLANLEKVLVIDYKPTTVRV

SEQ ID NO: 5 - protein, 82 aa, Citrus sinensis
MIAELIRSCCGLELLAVKYKGKNVSIENLHQGFTHIFESTFESTEGVAEYVAHPAHVEYANLFLANLEKVLVIDYKPTTVRV

SEQ ID NO: 6 - protein, 109 aa, Citrus sinensis
MEEAKGVVKHVLLAKFKEGTAQDQIDQLIKDYANLVNLIEPMKSFQWGKDVSIENRHQGFTHIFESTFESTEGVAEYVAHPAHVEYANLFLANLEKVLVIDYKPTTVRV

SEQ ID NO: 7 - protein, 114 aa, Citrus sinensis
MGEGEEAAMGEFKHLVIVKFKEGVVVEDIVKGMKKLVSEIDAVKSFEWGQDVEGQEMLRQGFTHAFLMTFNKKEDYTTFASHPSHVEFSATFSAAIEKIVLLDFPTVLGKAPAA

SEQ ID NO: 8 - protein, 118 aa, Citrus clementina
MKAETKGRDMEEAKGVVKHVLLAKFKEGTAQDQIDQLIKDYANLVNLIEPMKSFQWGKDVSIENLHQGFTHIFESTFESTEGVAEYVAHPAHVEYANLFLANLEKVLVIDYKPTTVRV

SEQ ID NO: 9 - protein, 67 aa, Capsicum annuum
MSYGRGKDVSTENLQQGFTHVFESTFDSTEGVAEYVSHPVHVEFANLMLPQLEKVLVIDYKPEKVGP

SEQ ID NO: 10 - protein, 107 aa, Nicotiana benthamiana
MEGGKVKHILLAKFKDGIPADQIDQLIKQYANLVNLIEPMKAFHWGENVSIENFHQGFTHVFESTFDSTEGIAEYIDHPAHVEYANTLLPQLEKVLVIDYKPEKVGP

SEQ ID NO: 11 - protein, 106 aa, Solanum lycopersicum
MEGGKGGVVKHILLAKFKDGIPPEQIDQLIKQYANLVNLVEPMKAFQWGKDVSIENLHQGFTHVFESTFDSLEGVAEYIAHPVHVEYANTLLPQLEKFLIVDYKPQ

SEQ ID NO: 12 - protein, 116 aa, Solanum melongena
MNIAVFLPSSCPALPRSKASRPSPPGQFPFLAKNVQLLLVLRSYSSTARAMSLRGENVSIENLHQGFTHVFESTFDSVEGIAEYIDHPAHVEYANILLTQLEKVLVIDYKPEKLSP

SEQ ID NO: 13 - protein, 106 aa, Solanum tuberosum
MEGGKGGVVKHILLAKFKDGIPPEQIDQLIKQYANLVNLIEPMKAFQWGKDVSIENLHQGFTHVFESTFDSLEGVAEYIAHPVHVEFANTMLPQLEKVLIIDYKPQ

SEQ ID NO: 14 - protein, 67 aa, Ponirus trifoliate
MCCNRGKNVSIENLHQGFTHIFESTFESTEGVAEYVAHPAHVEYANSFLANLEKVLVIDYKPTTVRV

SEQ ID NO: 15 - protein, 67 aa, Poncirus trifoliate
MCCNRGKNVSIENLHQGFTHIFESTFESTEGVAEYVAHPAHVEYTNSFLANLEKVLVIDYKPTTVRV

SEQ ID NO: 16 - protein, 67 aa, Unknown
  Description of Unknown SAMP sequence
MCCNRGKNVSIENLHQGFTHIFESTFESTEGVAEYVSHPAHVEYANLFLANLEKVLVIDYKPTTVRV

SEQ ID NO: 17 - protein, 30 aa, Unknown
  Description of Unknown SAMP sequence
HPSHVEYANLFLANLEKVLVIDYKPTTVRV

SEQ ID NO: 18 - protein, 30 aa, Unknown
  Description of Unknown SAMP sequence
HPAHVEYANLFLANLEKVLVIDYKPTTVRV

SEQ ID NO: 19 - protein, 30 aa, Unknown
  Description of Unknown SAMP sequence
HPAHVEYANLFLANLEKVLVIDYKPTTERV

SEQ ID NO: 20 - protein, 33 aa, Citrus sinensis
HPSHVEFSATFSAAIEKIVLLDFPTVLGKAPAA

SEQ ID NO: 21 - protein, 30 aa, Capsicum annuum
HPVHVEFANLMLPQLEKVLVIDYKPEKVGP

SEQ ID NO: 22 - protein, 30 aa, Nicotiana benthamiana
HPAHVEYANTLLPQLEKVLVIDYKPEKVGP

SEQ ID NO: 23 - protein, 26 aa, Solanum lycopersicum
HPVHVEYANTLLPQLEKFLIVDYKPQ

SEQ ID NO: 24 - protein, 30 aa, Solanum melongena
HPAHVEYANILLTQLEKVLVIDYKPEKLSP

SEQ ID NO: 25 - protein, 26 aa, Solanum tuberosum
HPVHVEFANTMLPQLEKVLIIDYKPQ

SEQ ID NO: 26 - protein, 30 aa, Ponirus trifoliate
HPAHVEYANSFLANLEKVLVIDYKPTTVRV

SEQ ID NO: 27 - protein, 30 aa, Poncirus trifoliate
HPAHVEYTNSFLANLEKVLVIDYKPTTVRV

SEQ ID NO: 28 - protein, 30 aa, Arabidopsis sp.
HPAHVEFATIFLGSLDKVLVIDYKPTSVSL

SEQ ID NO: 29 - protein, 31 aa, Oryza brachyantha
HPAHVEFANEFLPALEKTLIIDYKPTSGNNS

SEQ ID NO: 30 - protein, 30 aa, Prunus mume
HPAHVEFANLFLSHVEKVIVFDYKPTTVRV

SEQ ID NO: 31 - protein, 33 aa, Artificial Sequence
  Description of Artificial Sequence Synthetic polypeptide
  MOD_RES (3)..(3): S, A, or V
  MOD_RES (5)..(5): V or L
  MOD_RES (7)..(7): Y or F
  MOD_RES (8)..(8): A, S, or T
  MOD_RES (9)..(9): N, A, or T
  MOD_RES (10)..(10): L, T, I, S, or E
  MOD_RES (11)..(11): F, M, or L
  MOD_RES (12)..(12): L or S
  MOD_RES (13)..(13): A, P, T, G, or S
  MOD_RES (14)..(14): N, A, Q, S, or H
  MOD_RES (15)..(15): L, I, or V
  MOD_RES (16)..(16): E or D
  MOD_RES (18)..(18): V, I, F, or T
  MOD_RES (19)..(19): L, V, or I
  MOD_RES (20)..(20): V, L, or I
  MOD_RES (21)..(21): I, L, V, or F
  MOD_RES (23)..(23): Y or F
  MOD_RES (24)..(24): K or P
  MOD_RES (25)..(25): P or T
  MOD_RES (26)..(26): T, V, E, or Q
  MOD_RES (27)..(27): T, L, K, S, or absent
  MOD_RES (28)..(28): V, E, G, L, or absent
  MOD_RES (29)..(29): R, K, G, S, N, or absent
  MOD_RES (30)..(30): V, A, P, L, N, or absent
  MOD_RES (31)..(31): P, S, or absent
  MOD_RES (32)..(32): A or absent
  MOD_RES (33)..(33): A or absent
HPXHXEXXXXXXXXXXKXXXXDXXXXXXXXXXX

SEQ ID NO: 32 - protein, 31 aa, Artificial Sequence
  Description of Artificial Sequence Synthetic polypeptide
  MOD_RES (13)..(13): G, P, or S
  MOD_RES (14)..(14): S, A, or H
  MOD_RES (15)..(15): L or V
  MOD_RES (16)..(16): D or E
  MOD_RES (18)..(18): V or T
  MOD_RES (19)..(19): L or I
  MOD_RES (20)..(20): V or I
  MOD_RES (21)..(21): I or F
  MOD_RES (27)..(27): S or T
  MOD_RES (28)..(28): V or G
  MOD_RES (29)..(29): S, N, or R
  MOD_RES (30)..(30): L, N, or V
  MOD_RES (31)..(31): S or absent
HPAHVEFATIFLXXXXKXXXXDYKPTXXXXX

SEQ ID NO: 33 - protein, 33 aa, Artificial Sequence
  Description of Artificial Sequence Synthetic polypeptide
  MOD_RES (1)..(1): R, K, or W
  MOD_RES (3)..(3): K or E
  MOD_RES (4)..(4): N or D
  MOD_RES (7)..(7): T or I
  MOD_RES (10)..(10): L, F, or R
  MOD_RES (11)..(11): H or Q
  MOD_RES (15)..(15): P or T
  MOD_RES (17)..(17): I, L, or V
  MOD_RES (20)..(20): S or F
  MOD_RES (23)..(23): E or D
  MOD_RES (25)..(25): T or L
  MOD_RES (28)..(28): V or I
  MOD_RES (32)..(32): V or I
  MOD_RES (33)..(33): S, A, or D
XGXXVSXENXXQGFXHXFEXTFXSXEGXAEYXX

SEQ ID NO: 34 - protein, 22 aa, Unknown
  Description of Unknown SAMP sequence
SHVEYANLFLANLEKVLVIDYK

SEQ ID NO: 35 - protein, 22 aa, Unknown
  Description of Unknown SAMP sequence
AHVEYANLFLANLEKVLVIDYK

SEQ ID NO: 36 - protein, 22 aa, Unknown
  Description of Unknown SAMP sequence
AHVEYANLFLANLEKVLVIDYK

SEQ ID NO: 37 - protein, 22 aa, Unknown
  Description of Unknown SAMP sequence
SHVEFSATFSAAIEKIVLLDFP

SEQ ID NO: 38 - protein, 22 aa, Capsicum annuum
VHVEFANLMLPQLEKVLVIDYK

SEQ ID NO: 39 - protein, 22 aa, Nicotiana benthamiana
AHVEYANTLLPQLEKVLVIDYK

SEQ ID NO: 40 - protein, 22 aa, Solanum lycopersicum
VHVEYANTLLPQLEKFLIVDYK

SEQ ID NO: 41 - protein, 22 aa, Solanum melongena
AHVEYANILLTQLEKVLVIDYK

SEQ ID NO: 42 - protein, 22 aa, Solanum tuberosum
VHVEFANTMLPQLEKVLIIDYK

SEQ ID NO: 43 - protein, 22 aa, Poncirus trifoliate
AHVEYANSFLANLEKVLVIDYK

SEQ ID NO: 44 - protein, 22 aa, Poncirus trifoliate
AHVEYTNSFLANLEKVLVIDYK

SEQ ID NO: 45 - protein, 22 aa, Unknown
  Description of Unknown SAMP sequence
AHVEFATIFLGSLDKVLVIDYK

SEQ ID NO: 46 - protein, 22 aa, Unknown
  Description of Unknown SAMP sequence
AHVEFANEFLPALEKTLIIDYK

SEQ ID NO: 47 - protein, 22 aa, Unknown
  Description of Unknown SAMP sequence
AHVEFANLFLSHVEKVIVFDYK

SEQ ID NO: 48 - protein, 67 aa, Murraya paniculata
MCCNRGKNVSVENLHQGFTHIFESTFESTEGVAEYVAHPVHLEFANLFLANLEKVLVIDYKPTTVRV

SEQ ID NO: 49 - protein, 30 aa, Murraya paniculata
HPVHLEFANLFLANLEKVLVIDYKPTTVRV

SEQ ID NO: 50 - protein, 22 aa, Murraya paniculata
VHLEFANLFLANLEKVLVIDYK

SEQ ID NO: 51 - protein, 23 aa, Citrus sinensis
SHVEFSATFSAAIEKIVLLDFPT

SEQ ID NO: 52 - protein, 22 aa, Poncirus trifoliate
AHVEYANSFLANLDKVLVIDYK

SEQ ID NO: 53 - protein, 22 aa, Artificial Sequence
  Description of Artificial Sequence Synthetic peptide
  MOD_RES (1)..(1): V, S, or A
  MOD_RES (3)..(3): V or L
  MOD_RES (5)..(5): F or Y
  MOD_RES (6)..(6): A, T, or S
  MOD_RES (7)..(7): N, A, or T
  MOD_RES (8)..(8): L, E, I, S, or T
  MOD_RES (9)..(9): F, L, or M
  MOD_RES (10)..(10): L or S
  MOD_RES (11)..(11): A, S, P, G, or T
  MOD_RES (12)..(12): N, A, Q, or S
  MOD_RES (13)..(13): L, V, or I
  MOD_RES (14)..(14): E or D
  MOD_RES (16)..(16): V, I, T, or F
  MOD_RES (17)..(17): L, I, or V
  MOD_RES (18)..(18): V, L, or I
  MOD_RES (19)..(19): I, L, V, or F
XHXEXXXXXXXXXXKXXXXDYK

SEQ ID NO: 54 - protein, 22 aa, Artificial Sequence
  Description of Artificial Sequence Synthetic peptide
  MOD_RES (1)..(1): V, S, or A
  MOD_RES (3)..(3): V or L
  MOD_RES (5)..(5): F or Y
  MOD_RES (6)..(6): A, T, or S
  MOD_RES (8)..(8): L, E, I, S, or T
  MOD_RES (9)..(9): F, L, or M
  MOD_RES (10)..(10): L or S
  MOD_RES (11)..(11): A, S, P, G, or T
  MOD_RES (12)..(12): N, A, Q, or S
  MOD_RES (13)..(13): L, V, or I
  MOD_RES (14)..(14): E or D
  MOD_RES (16)..(16): V, I, or T
  MOD_RES (17)..(17): L, I, or V
  MOD_RES (18)..(18): V, L, or I
  MOD_RES (19)..(19): I, L, or V
XHXEXXNXXXXXXXKXXXXDYK

SEQ ID NO: 55 - protein, 22 aa, Artificial Sequence
  Description of Artificial Sequence Synthetic peptide
  MOD_RES (1)..(1): V, S, or A
  MOD_RES (5)..(5): F or Y
  MOD_RES (8)..(8): L, I, S, or T
  MOD_RES (9)..(9): F or L
  MOD_RES (10)..(10): L or S
  MOD_RES (11)..(11): A or P
  MOD_RES (12)..(12): N, A, Q, or S
  MOD_RES (17)..(17): L or I
  MOD_RES (18)..(18): V, L, or I
  MOD_RES (19)..(19): I, L, or V
XHVEXANXXXXXLEKVXXXDYK

SEQ ID NO: 56 - protein, 22 aa, Artificial Sequence
  Description of Artificial Sequence Synthetic peptide
  MOD_RES (1)..(1): V, S, or A
  MOD_RES (5)..(5): F or Y
  MOD_RES (8)..(8): L, I, S, or T
  MOD_RES (9)..(9): F or L
  MOD_RES (12)..(12): N or Q
  MOD_RES (17)..(17): L or I
  MOD_RES (18)..(18): V, L, or I
  MOD_RES (19)..(19): I, L, or V
XHVEXANXXLAXLEKVXXXDYK

SEQ ID NO: 57 - protein, 42 aa, Unknown
  Description of Unknown SAMP sequence
HIFESTFESTEGVAEYVSHPSHVEYANLFLANLEKVLVIDYK

SEQ ID NO: 58 - protein, 21 aa, Unknown
  Description of Unknown SAMP sequence
THIFESTFESTEGVAEYVSHP

SEQ ID NO: 59 - protein, 18 aa, Unknown
  Description of Unknown SAMP sequence
PSHVEYANLFLANLEKVL

SEQ ID NO: 60 - protein, 6 aa, Artificial Sequence
  Description of Artificial Sequence Synthetic 6xHis tag
HHHHHH